SEQUENCE LISTING (67 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 70 aa, Artificial Sequence
  synthetic construct
GPRTLCGAELVDALQFVCGDRGFYFNKPTGAGSSSRRAPQTGIVDECCFRSCDLRRLEMYCAPLKPAKSA

SEQ ID NO: 3 - protein, 83 aa, Artificial Sequence
  synthetic construct
MFPAMPLSSLFVNGPETLCGAELVDALQFVCGDRGFYFNKPTGYGSSSRRAPQTGIVDECCFRSCDLRRLEMYCAPLKPAKSA

SEQ ID NO: 4 - protein, 70 aa, Artificial Sequence
  synthetic construct
GPRTLCGAELVDALQFVCGDRGFYFNKPTGYGSSSRRAPQTGIVDECCFRSCDLRRLEMYCAPLKPAKSA

SEQ ID NO: 5 - protein, 67 aa, Artificial Sequence
  synthetic construct
TLCGAELVDALQFVCGDRGFYFNKPTGYGSSSRRAPQTGIVDECCFRSCDLRRLEMYCAPLKPAKSA

SEQ ID NO: 6 - protein, 83 aa, Artificial Sequence
  synthetic construct
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 7 - protein, 69 aa, Artificial Sequence
  synthetic construct
GPETLCGAELVDALQFVCGDRGFYFNKPTGYGSSSRAPQTGIVDECCFRSCDLRRLEMYCAPLKPAKSA

SEQ ID NO: 8 - protein, 67 aa, Artificial Sequence
  synthetic construct
GPETLCGAELVDALQFVCGDRGFYFNKPTGYGSSSRRAPQTGIVDECCFRSCDLRRLEMYCAPLKPA

SEQ ID NO: 9 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 10 - protein, 131 aa, Homo sapiens
GHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAK

SEQ ID NO: 11 - protein, 180 aa, Homo sapiens
ALGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQ

SEQ ID NO: 12 - protein, 585 aa, Homo sapiens [sequence omitted]

SEQ ID NO: 13 - protein, 585 aa, Artificial Sequence
  synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGL

SEQ ID NO: 14 - protein, 585 aa, Artificial Sequence
  synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAGTFTFHADICTLSEKERQIKKQTALVELVKHKPKATKEQLKAAMDDFAAFVEKCCKADDKETCFAEEGKKLVAASQAALGL

SEQ ID NO: 15 - protein, 585 aa, Artificial Sequence; identical to SEQ ID NO: 13
  synthetic construct

SEQ ID NO: 16 - protein, 20 aa, Artificial Sequence
  synthetic construct
GGGGSGGGGSGGGGSGGGGS

SEQ ID NO: 17 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 18 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 19 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 20 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 21 - protein, 20 aa, Artificial Sequence
  synthetic construct
ASPAAPAPASPAAPAPSAPA

SEQ ID NO: 22 - protein, 107 aa, Artificial Sequence
  synthetic construct
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIK

SEQ ID NO: 23 - protein, 7 aa, Artificial Sequence
  synthetic construct
GSGGGSG

SEQ ID NO: 24 - protein, 141 aa, Artificial Sequence
  synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 25 - protein, 70 aa, Artificial Sequence
  Synthetic construct
GPRTLCGAELVDALQFVCGDRGFLFNKPTGYGSSSRRAPQTGIVDECCFRSCDLRRLEMYCAPLKPAKSA

SEQ ID NO: 26 - protein, 70 aa, Artificial Sequence
  Synthetic construct
GPRTLCGAELVDALQFVCGDRGFLFNKPTGYGSSSRRAPQTGIVDECCFRSCDLRRLEMLCAPLKPAKSA

SEQ ID NO: 27 - protein, 70 aa, Artificial Sequence
  Synthetic construct
GPRTLCGAELVDALQFVCGDRGFLFNKPTGAGSSSRRAPQTGIVDECCFRSCDLRRLEMYCAPLKPAKSA

SEQ ID NO: 28 - protein, 70 aa, Artificial Sequence; identical to SEQ ID NO: 26
  Synthetic construct

SEQ ID NO: 29 - protein, 70 aa, Artificial Sequence
  Synthetic construct
GPRTLCGAELVDALQFVCGDRGFYFNKPTGAGSSSRRAPQTGIVDECCFRSCDLRRLEMLCAPLKPAKSA

SEQ ID NO: 30 - protein, 70 aa, Artificial Sequence
  Synthetic construct
GPRTLCGAELVDALQFVCGDRGFLFNKPTGAGSSSRRAPQTGIVDECCFRSCDLRRLEMLCAPLKPAKSA

SEQ ID NO: 31 - protein, 72 aa, Homo sapiens
GTSHLVKCAEKEKTFCVNGGECFMVKDLSNPSRYLCKCPNEFTGDRCQNYVMASFYKHLGIEFMEAEELYQK

SEQ ID NO: 32 - protein, 72 aa, Artificial Sequence
  Synthetic construct
STSHLVKCAEKEKTFCVNGGECFMVKDLSNPSRYLCKCPNEFTGDRCQNYVMASFYKHLGIEFMEAEELYQK

SEQ ID NO: 33 - protein, 68 aa, Homo sapiens
TGTSHLVKCAEKEKTFCVNGGECFMVKDLSNPSRYLCKCQPGFTGARCTENVPMKVQNQEKAEELYQK

SEQ ID NO: 34 - protein, 76 aa, Homo sapiens
TGTSHLVKCAEKEKTFCVNGGECFMVKDLSNPSRYLCKCQPGFTGARCTENVPMKVQNQEKHLGIEFIEAEELYQK

SEQ ID NO: 35 - protein, 74 aa, Homo sapiens
TGTSHLVKCAEKEKTFCVNGGECFMVKDLSNPSRYLCKCQPGFTGARCTENVPMKVQNQESAQMSLLVIAAKTT

SEQ ID NO: 36 - protein, 65 aa, Homo sapiens
TGTSHLVKCAEKEKTFCVNGGECFMVKDLSNPSRYLCKCPNEFTGDRCQNYVMASFYKAEELYQK

SEQ ID NO: 37 - protein, 68 aa, Homo sapiens
TGTSHLVKCAEKEKTFCVNGGECFMVKDLSNPSRYLCKCPNEFTGDRCQNYVMASFYSTSTPFLSLPE

SEQ ID NO: 38 - protein, 114 aa, Homo sapiens
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 39 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 40 - protein, 114 aa, Homo sapiens
TGAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRAR

SEQ ID NO: 41 - protein, 1080 aa, Artificial Sequence
  Synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGASALTQPSSVSANPGGTVEITCSGGGRYYDGSYYYGWYQQKSPGSAPVTVIYENTKRPSNIPSRFSGSKSGSTATLTITGVRAEDEAVYYCGSADDNMNPTIFGAGTTLTVLGGSSRSSGGGGSGGGGSSALTLDESGGGLQTPGGALSLVCKASGFTFSSYGMQWVRQAPGKGLEWVAGIQNDDTGTYYGAAVKGRATISRDNGQSTVRLQLNNLRAEDTGTYYCAKDASSDGGYGGDSIDAWGHGTEVIVSSTSGQAGQHHHHHHGAYPYDVPDYAS

SEQ ID NO: 42 - protein, 1051 aa, Artificial Sequence
  Synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGASQVQLQQSGGGLVQPGRSLRLSCEASGFTFSSYAMSWVRQAPGKGLEWVSAISGSGGSTYYADSVKGRFTISRDNSKNTLYLQMNSLRAEDTAVYYCAKPLGDLYVNDAFDIWGQGTMVTVSSGGGGSGGGGSGGGGSQSVLTQPPSVSGAPGQRVTISCTGSSSNIGAGYDVHWYQQLPGTAPKLMIYEVSNRPSGVSNRFSGSKSGNTASLTISGLQTEDEAYYYCSSYASGNIVVFGGGTKLTVLG

SEQ ID NO: 43 - protein, 1044 aa, Artificial Sequence
  Synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGASQVQLVESGGGLVQPGGSLRLSCTASGFTFRSYSMNWVRQAPGKGLEWVAYITNSSSTILYADSVKGRFTISRDNVKNSLYLQMNSLRDEDTAVYYCATAKLDHWGQGTLVTVSSGGGGSGGGGSGGGGSHVILTQPPSVSGAPGQRVTISCTGSSSNIGAGYDVHWYQQLPGTAPKLLIYRNSNRPSGVPDRFSGSKSGSSASLAITGLQAEDEADYYCQSYDNSLTALVFGGGTKVTVLG

SEQ ID NO: 44 - protein, 1064 aa, Artificial Sequence
  Synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGASQVQLLESGGGLVQPGGSLRLSCAASGFSFNNYAMTWVRQAPGKGLEWVSVISDGGAGTYYADSVKGRFTISRDNSKNTLYLQMNSLRAEDTAVYYCARTSYVYEYQQWTRGSDYWGQGTLVTVSSASSGGSTSGSGKPGSGEGSSGSARQSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLG

SEQ ID NO: 45 - protein, 1056 aa, Artificial Sequence
  Synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGASQVQLLESGGGLVQPGGSLRLSCAASGFTFTNYAMSWVRQAPGKGLEWVSGISGGGGRTYYADSVKGRFTISRDNSKNTLYLQMNSLRAEDTAVYYCARTPGSYPFDYWGQGTLVTVSSASSGGSTSGSGKPGSGEGSSGSARQSALTQPASVSGSPGQSITISCTGTSNDIGAYDYVSWYQQHPGKAPKLMIYEVTKRPSGVSNRFSGSKSGNTASLTISGLQAEDEADYYCSSYTSTNIFVFGGGTKLTVLG

SEQ ID NO: 46 - protein, 1044 aa, Artificial Sequence
  Synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGASQVQLQQSGGGLVQPGGSLRLSCAASGFTFSIYAMSWVRQAPGKGLEWVSAISDSGTGTYYADSVKGRFTISRDNSKNTLYLQMNSLTAEDTAVYYCARRSGNYYLDYWGQGTLVTVSSGGGGSGGGGSGGGGSDIVMTQSPSTLSASIGDRVTITCRASQSISSWLAWYQQKPGKAPKLLIHKTSNLESGVPSRFSGSGSGTEFSLTISSLQPDDFATYYCQQYYSYPWTFGRGTKLEIKR

SEQ ID NO: 47 - protein, 662 aa, Artificial Sequence
  Synthetic construct
GPRTLCGAELVDALQFVCGDRGFYFNKPTGYGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGL

SEQ ID NO: 48 - protein, 7 aa, Artificial Sequence
  Synthetic construct
AAALAAA

SEQ ID NO: 49 - protein, 15 aa, Artificial Sequence
  Synthetic construct
GSGGSGGGSGGSGGG

SEQ ID NO: 50 - protein, 40 aa, Artificial Sequence
  Synthetic construct
GSGGSGGGSGGGSGGGSGGGSGGGSGGGSGGGSGGGSGGG

SEQ ID NO: 51 - protein, 141 aa, Artificial Sequence; identical to SEQ ID NO: 24
  Synthetic construct

SEQ ID NO: 52 - protein, 245 aa, Artificial Sequence
  Synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGAS

SEQ ID NO: 53 - protein, 349 aa, Artificial Sequence
  Synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 54 - protein, 451 aa, Artificial Sequence
  Synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAGGGGSGGGGSGGGGAS

SEQ ID NO: 55 - protein, 733 aa, Artificial Sequence
  Synthetic construct
GSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 56 - protein, 837 aa, Artificial Sequence
  Synthetic construct
GSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGAS

SEQ ID NO: 57 - protein, 1533 aa, Artificial Sequence
  Synthetic construct
GSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 58 - protein, 1043 aa, Artificial Sequence
  Synthetic construct
GSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLKLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAGGGGSGGGGSGGGGAS

SEQ ID NO: 59 - protein, 4 aa, Artificial Sequence
  Synthetic construct
GGGS

SEQ ID NO: 60 - protein, 5 aa, Artificial Sequence
  Synthetic construct
GGGGS

SEQ ID NO: 61 - protein, 4 aa, Artificial Sequence
  Synthetic construct
GGSG

SEQ ID NO: 62 - protein, 70 aa, Artificial Sequence
  Synthetic construct
GPKTLCGAELVDALQFVCGDRGFYFNKPTGYGSSSRRAPQTGIVDECCFRSCDLRRLEMYCAPLKPAKSA

SEQ ID NO: 63 - protein, 70 aa, Artificial Sequence
  Synthetic construct
GPKTLCGAELVDALQFVCGDRGFLFNKPTGYGSSSRRAPQTGIVDECCFRSCDLRRLEMYCAPLKPAKSA

SEQ ID NO: 64 - protein, 70 aa, Artificial Sequence
  Synthetic construct
GPKTLCGAELVDALQFVCGDRGFYFNKPTGAGSSSRRAPQTGIVDECCFRSCDLRRLEMYCAPLKPAKSA

SEQ ID NO: 65 - protein, 70 aa, Artificial Sequence
  Synthetic construct
GPKTLCGAELVDALQFVCGDRGFYFNKPTGYGSSSRRAPQTGIVDECCFRSCDLRRLEMLCAPLKPAKSA

SEQ ID NO: 66 - protein, 70 aa, Artificial Sequence
  Synthetic construct
GPKTLCGAELVDALQFVCGDRGFLFNKPTGYGSSSRRAPQTGIVDECCFRSCDLRRLEMLCAPLKPAKSA

SEQ ID NO: 67 - protein, 70 aa, Artificial Sequence
  Synthetic construct
GPKTLCGAELVDALQFVCGDRGFLFNKPTGAGSSSRRAPQTGIVDECCFRSCDLRRLEMLCAPLKPAKSA

(19) United States
(12) Patent Application Publication
(10) Pub. No.: US 2024/0279359 A1
(43) Pub. Date: Aug. 22, 2024

(54) CHIMERIC PROTEINS FOR TARGETED DELIVERY OF GROWTH FACTORS TO THE GLOMERULUS

(71) Applicant: Silver Creek Pharmaceuticals, Inc., San Francisco, CA (US)

(72) Inventors: Kristopher M. Kuchenbecker, Phoenix, AZ (US); Walter Olsen, Dracut, MA (US); Samuel J. Pfaff, Wyncote, PA (US); Yan Zhang, Sausalito, CA (US); Lakhmir S. Chawla, San Diego, CA (US)

(73) Assignee: Silver Creek Pharmaceuticals, Inc., San Francisco, CA (US)

(21) Appl. No.: 18/571,806

(22) PCT Filed: Jun. 30, 2022
(86) PCT No.: PCT/US2022/035844

§ 371 Date: Dec. 19, 2023

Related U.S. Application Data
(60) Provisional application No. 63/216,876, filed on Jun. 30, 2021.

(51) Int. Cl. C07K 16/40; A61K 38/00; C07K 14/50; C07K 14/51; C07K 14/705; C07K 16/28

(52) U.S. Cl. CPC C07K 16/40; C07K 14/50; C07K 14/51; C07K 14/705; C07K 16/28; A61K 38/00; C07K 2317/622; C07K 2319/31; C07K 2319/33

(57) ABSTRACT

Aspects of the present disclosure relate generally to chimeric proteins and pharmaceutical compositions comprising such chimeric proteins, and methods for using such chimeric proteins for treating kidney diseases. Aspects of the disclosure relate to chimeric protein comprising a targeting domain comprising a scFv having a binding specificity to a cell surface protein of a kidney tissue, and an activator domain comprising a growth factor.

### RELATED APPLICATIONS

This application claims priority to and the benefit of U.S. Provisional Application No. 63/216,876, filed Jun. 30, 2021, the content of which is incorporated herein by reference in its entirety.

### REFERENCE TO SEQUENCE LISTING

This specification includes a sequence listing submitted herewith, which includes the file entitled 132463-010701_ST25.txt having the following size: 174,728 bytes which was created Jun. 30, 2022, the contents of which are incorporated by reference herein.

### TECHNICAL FIELD

Aspects of the present disclosure relates generally to chimeric proteins and pharmaceutical compositions comprising such chimeric proteins, and methods for using such chimeric proteins for treating kidney diseases, such as glomerular disorders.

### BACKGROUND

Many diseases or disorders affect kidney function by attacking the glomeruli. These diseases have a variety of genetic and environmental causes.

### SUMMARY

Aspects of the disclosure relate to a chimeric protein comprising a targeting domain comprising a scFv having a binding specificity to a cell surface protein of a kidney tissue, and an activator domain comprising a growth factor, wherein the targeting domain is at the C-terminus and the activator domain is at the N-terminus of the chimeric protein. Other aspects of the disclosure relate to a chimeric protein comprising a targeting domain comprising a scFv having a binding specificity to a cell surface protein of a kidney tissue, and an activator domain comprising a growth factor, wherein the targeting domain is at the N-terminus and the activator domain is at the C-terminus of the chimeric protein.

In some embodiments, the chimeric protein further comprises one or more peptide linkers between the targeting domain and the activator domain. In some embodiments, the chimeric protein further comprises a half-life modulator between the targeting domain and the activator domain. In some embodiments, the chimeric protein further comprises a peptide linker between the targeting domain and the half-life modulator. In some embodiments, the peptide linker comprises or consists of a tetratricopeptide repeat (TPR) domain. In some embodiments, the chimeric protein further comprises a peptide linker between the activator domain and half-life modulator. In some embodiments, the half-life modulator extends the half-life of the chimeric protein.

In some embodiments, the targeting domain targets cell surface protein of the glomerulus. In some embodiments, the targeting domain has a binding specificity to a protein displayed at the cell surface of podocytes, parietal epithelial cells, or glycocalyx-coated fenestrated endothelial cells of the kidney. In some embodiments, the targeting domain has a binding specificity to Protein Tyrosine Phosphatase Receptor Type O (PTPRO), Podocin, Phospholipase A2 Receptor 1, Integrin Subunit alpha 8, Apoptosis resistant E3 ubiquitin protein ligase 1, Integrin Subunit alpha 8, Apoptosis resistant E3 ubiquitin protein ligase 1, Podocalyxin, Synaptopodin, Alpha 3 integrin, Cysteine rich transmembrane BMP regulator 1, FAT atypical cadherin 1, NEPH1, Dystroglycan, or Podoplanin.

In some embodiments, the activator domain comprises or consists of IGF-1, IGF-2, Neuregulin1α, Neuregulin1β, BMP2, BMP7, FGF2, FGF9 VEGF-A or variant thereof or fragment thereof.

In some embodiments, the chimeric protein comprises a scFv having a binding specificity to Protein Tyrosine Phosphatase Receptor Type O, and a variant of human insulin-like growth factor IGF-1, wherein the variant of IGF-1 comprises one or more mutations, wherein the one or more mutations consist of a substitution at one or more positions corresponding to E3, Y24, Y31, Y60, and combinations thereof, wherein the scFv is at the C-terminus and the variant of human insulin-like growth factor IGF-1 is at the N-terminus of the chimeric protein.

In some embodiments, the chimeric protein comprises (a) a scFv having a binding specificity to Protein Tyrosine Phosphatase Receptor Type O, (b) a variant of human insulin-like growth factor IGF-1, wherein the variant of IGF-1 comprises one or more mutations, wherein the one or more mutations comprise or consist of a substitution at one or more positions corresponding to E3, Y24, Y31, Y60, and combinations thereof, and (c) a variant of human serum albumin (HSA), wherein the variant of HSA comprises one or more mutations, wherein the one or more mutations comprise or consist of a substitution at one or more positions corresponding to C58 and N527, and combinations thereof, wherein the scFv is at the C-terminus and the variant of human insulin-like growth factor IGF-1 is at the N-terminus of the chimeric protein.

In some embodiments, the chimeric protein comprises (a) a variant of human insulin-like growth factor IGF-1, wherein the variant of IGF-1 comprises one or more mutations, wherein the one or more mutations comprise or consist of a substitution at one or more positions corresponding to E3, Y24, Y31, Y60, and combinations thereof at the N-terminus of the chimeric protein, (b) a first peptide linker at the C-terminus of the variant of human insulin-like growth factor IGF-1, (c) a variant of human serum albumin (HSA) at the C-terminus of the first peptide linker, wherein the variant of HSA comprises one or more mutations, wherein the one or more mutations comprise or consist of a substitution at one or more positions corresponding to C58 and N527, and combinations thereof, (d) a second peptide linker at the C-terminus of the variant of human serum albumin, and (e) a scFv having a binding specificity to Protein Tyrosine Phosphatase Receptor Type O is at the C-terminus of the chimeric protein. In some embodiments, the first linker comprises or consists of amino acid sequence (GSGGGSG)1-7 and wherein the second linker comprises or consists of a tetratricopeptide repeat (TPR) domain.

Aspects of the disclosure relate to a pharmaceutical composition comprising the chimeric protein and at least one physiologically acceptable carrier.

Aspects of the disclosure relate to a method for treating kidney disease comprising administering the chimeric protein or the pharmaceutical composition to a subject in need thereof. In some embodiments, the kidney disease is glomerulonephritis, nephrotic syndrome, chronic kidney disease, diabetic nephropathy or acute kidney injury. In some embodiments, the chimeric protein or the pharmaceutical composition is administered intravenously, subcutaneously, or intraperitoneally to the subject in need thereof. In some embodiments, the administering results in improvement of the subject's kidney function as assessed by serum creatinine and blood urea nitrogen levels.

Aspects of the disclosure relates to a polypeptide domain comprising a half-life modulator with a first peptide linker at its N-terminus and a second peptide linker at its C-terminus, wherein the first peptide linker comprises or consists of an amino acid sequence consisting of glycine and/or serine, wherein the second linker comprises or consists of a tetratricopeptide repeat (TPR) domain. In some embodiments, the half-life modulator is variant of human serum albumin (HSA). In some embodiments, the human serum albumin comprises or consists of amino acid sequence set forth in SEQ ID NOs: 13-15. In some embodiments, the first peptide linker comprises or consists of amino acid sequence set forth in SEQ ID NO: 23. In some embodiments, the second peptide linker comprises or consists of amino acid sequence set forth in SEQ ID NO: 24, SEQ ID NO: 51, SEQ ID NO: 52, SEQ ID NO: 53 or SEQ ID NO: 54. In some embodiments, the polypeptide domain comprises an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence set forth SEQ ID NO: 55, SEQ ID NO: 56, SEQ ID NO: 57, or SEQ ID NO: 58.

Aspects of the disclosure relates to a polypeptide domain comprising a half-life modulator with a first peptide linker at its N-terminus and a second peptide linker at its C-terminus, wherein the first peptide linker comprises or consists of a tetratricopeptide repeat (TPR) domain, wherein the second linker comprises or consists of an amino acid sequence consisting of glycine and/or serine. In some embodiments, the half-life modulator is variant of human serum albumin (HSA). In some embodiments, the HSA comprises or consists of amino acid sequence set forth in SEQ ID NOs: 13-15. In some embodiments, the the second peptide linker comprises or consists of amino acid sequence set forth in SEQ ID NO: 23. In some embodiments, the first peptide linker comprises or consists of amino acid sequence set forth in SEQ ID NO: 24, SEQ ID NO: 51, SEQ ID NO: 52, SEQ ID NO: 53 or SEQ ID NO: 54.

### DESCRIPTION OF THE SEQUENCE LISTING

SEQ ID NO: 1 is the amino acid sequence of wild-type human IGF-1 (mature form).
SEQ ID NO: 2 is the amino acid sequence of a variant of wild-type human IGF-1 variant comprising E3R and Y31A substitutions.
SEQ ID NO: 3 is the amino acid sequence of a variant of human IGF-1 (IGF-1 LONG).
SEQ ID NO: 4 is the amino acid sequence of a variant of human IGF-1 (IGF1 E3R).
SEQ ID NO: 5 is the amino acid sequence of a variant of human IGF-1(IGF-1Des 1-3).
SEQ ID NO: 6 is the amino acid sequence of a variant of human IGF-1 (IGF-1 LR3).
SEQ ID NO: 7 is the amino acid sequence of a variant of human IGF-1 (IGF1 R37X).
SEQ ID NO: 8 is the amino acid sequence of a variant of human IGF-1 with deletion of residues 68-70 (IGF1 3X).
SEQ ID NO: 9 is the amino acid sequence of a wild type human IGF-2 (mature form).
SEQ ID NO: 10 is the amino acid sequence of wild type human FGF2.
SEQ ID NO: 11 is the amino acid sequence of wild type human FGF9.
SEQ ID NO: 12 is the amino acid sequence of wild type Human Serum Albumin (HSA).
SEQ ID NO: 13 is the amino acid sequence of Human Serum Albumin variant mHSA (C34S, N503Q substitutions).
SEQ ID NO: 14 is the amino acid sequence of Human Serum Albumin variant mHSA7 (C34S, N503Q, E505G and V547A substitutions).
SEQ ID NO: 15 is the amino acid sequence of a variant of human serum albumin comprising the amino acids 26-609 of wild type human serum albumin and the C58S and N527Q substitutions.
SEQ ID NO: 16 is the amino acid sequence of a peptide linker.
SEQ ID NO: 17 is the amino acid sequence of human transferrin (Tf).
SEQ ID NO: 18 is the amino acid sequence of Human Alpha Fetoprotein (AFP).
SEQ ID NO: 19 is the amino acid sequence of Human Vitamin D Binding Protein (VDBP).
SEQ ID NO: 20 is the amino acid sequence of Human Transthyretin (TTR).
SEQ ID NO: 21 is the amino acid sequence of a PASylation motif.
SEQ ID NO: 22 is the amino acid sequence of the albumin-binding domain human antibody (aldudAB).
SEQ ID NO: 23 is the amino acid sequence of a peptide linker 1k7.
SEQ ID NO: 24 is the amino acid sequence of a peptide linker tetratricopeptide repeat (TPR) domain.
SEQ ID NO: 25 is the amino acid sequence of a variant of human IGF-1 (IGF-1 E3R, Y24L).
SEQ ID NO: 26 is the amino acid sequence of a variant of human IGF-1 (IGF-1 E3R, Y60L).
SEQ ID NO: 27 is the amino acid sequence of a variant of human IGF-1 (IGF-1 E3R, Y24L, Y31A).
SEQ ID NO: 28 is the amino acid sequence of a variant of human IGF-1 (IGF-1 E3R, Y24L, Y60L).
SEQ ID NO: 29 is the amino acid sequence of a variant of human IGF-1 (IGF-1 E3R, Y31A, Y60L).
SEQ ID NO: 30 is the amino acid sequence of a variant of human IGF-1 (IGF-1 E3R, Y24L, Y31A, Y60L).
SEQ ID NO: 31 is the amino acid sequence of wild type human neuregulin Beta1.
SEQ ID NO: 32 is the amino acid sequence of a variant of human neuregulin Beta1 (G1S substitution).
SEQ ID NO: 33 is the amino acid sequence of wild type human neuregulin Alpha.
SEQ ID NO: 34 is the amino acid sequence of wild type human neuregulin Alpha1A.
SEQ ID NO: 35 is the amino acid sequence of wild type human neuregulin Alpha3.
SEQ ID NO: 36 is the amino acid sequence of wild type human neuregulin Beta2.
SEQ ID NO: 37 is the amino acid sequence of wild type human Glian growth factor GGF2 (isoform of neuregulin 1Beta).
SEQ ID NO: 38 is the amino acid sequence of wild type human BMP2 (mature form).
SEQ ID NO: 39 is the amino acid sequence of wild type human BMP7 (mature form).
SEQ ID NO: 40 is the amino acid sequence of wild type human VEGFa (mature form).
SEQ ID NO: 41 is the amino acid sequence of scp801.
SEQ ID NO: 42 is the amino acid sequence of scp802.
SEQ ID NO: 43 is the amino acid sequence of scp803.
SEQ ID NO: 44 is the amino acid sequence of scp804.
SEQ ID NO: 45 is the amino acid sequence of scp805.
SEQ ID NO: 46 is the amino acid sequence of scp806.
SEQ ID NO: 47 is the amino acid sequence of scp207.
SEQ ID NO: 48 is the amino acid sequence of aliphatic linker 1k7.
SEQ ID NO: 49 is the amino acid sequence of linker 1k15.
SEQ ID NO: 50 is the amino acid sequence of linker 1k40.
SEQ ID NO: 51 is the amino acid sequence of linker cTPR3.
SEQ ID NO: 52 is the amino acid sequence of linker cTPR6.
SEQ ID NO: 53 is the amino acid sequence of linker cTPR9.
SEQ ID NO: 54 is the amino acid sequence of linker cTPR12.
SEQ ID NO: 55 is the amino acid sequence of 1k7-Human Serum Albumin—cTPR3.
SEQ ID NO: 56 is the amino acid sequence of 1k7-Human Serum Albumin—cTPR6.
SEQ ID NO: 57 is the amino acid sequence of 1k7-Human Serum Albumin—cTPR9.
SEQ ID NO: 58 is the amino acid sequence of 1k7-Human Serum Albumin—cTPR12.
SEQ ID NO: 59 is the amino acid sequence of a linker.
SEQ ID NO: 60 is the amino acid sequence of a linker.
SEQ ID NO: 61 is the amino acid sequence of a linker.
SEQ ID NO: 62 is the amino acid sequence of a variant of human IGF-1 (IGF-1 E3K).
SEQ ID NO: 63 is the amino acid sequence of a variant of human IGF-1 (IGF-1 E3K, Y24L).
SEQ ID NO: 64 is the amino acid sequence of a variant of human IGF-1 (IGF-1 E3K, Y31A).
SEQ ID NO: 65 is the amino acid sequence of a variant of human IGF-1 (IGF-1 E3K, Y60L).
SEQ ID NO: 66 is the amino acid sequence of a variant of human IGF-1(IGF-1 E3K, Y24L, Y60L).
SEQ ID NO: 67 is the amino acid sequence of a variant of human IGF-1(IGF-1 E3K, Y24L, Y31A, Y60L).

### BRIEF DESCRIPTION OF THE DRAWINGS

FIG. 1A shows the structure of the exemplary chimeric protein according to embodiments of the disclosure.

FIG. 1B shows the structure of the exemplary chimeric protein according to embodiments of the disclosure.

FIG. 1C shows the structure of the exemplary polypeptide domain according to embodiments of the disclosure.

FIG. 1D shows the structure of the exemplary polypeptide domain according to embodiments of the disclosure.

FIG. 2A shows the structure of the exemplary chimeric protein (Prototype Sequence Scp801) according to embodiments of the disclosure.

FIG. 2B shows the amino acid sequence of Prototype Sequence (Scp801) according to embodiments of the disclosure.

FIG. 3 shows the expression and purification of prototype sequence (Scp801).

FIG. 4 shows detection of different fusion proteins in the cortex of healthy rat kidney using anti-HSA immunohistochemistry (IHC) staining for scp207 (IGF-1 E3R-HSA), scp802 (IGF-1 E3RIY31A-HSA-anti-Ptpro), and scp806 (IGF-1 E3R/Y31A-HSA-anti-Neph1). The cortex is shown at 200× magnification in the bottom row (Wide Field Kidney Cortex), while representative images of proximal tubules and individual glomeruli are shown at 1,200× magnification in the top row (Representative Proximal Tubules) and middle rows (Representative Glomeruli) respectively. For the non-targeted control, diffuse staining is evident outside of the glomeruli. For the targeted growth factors, regions outside of the glomeruli are less intensely stained; the relatively intense staining of the glomeruli for the targeted growth factors reveals preferential distribution of these constructs to the glomeruli, consistent with known expression of these target molecules (PTPRO and Neph1).

FIG. 5 is a schematic of an exemplary chimeric protein according to embodiments of the disclosure.

FIG. 6 is a schematic of an exemplary polypeptide domain according to embodiments of the disclosure.

FIG. 7 shows the hydrophobic core positions of IGF-1.

### DETAILED DESCRIPTION

It is to be understood that the terminology used herein is for the purpose of describing particular embodiments of the disclosure only and is not intended to be limiting.

Unless defined otherwise, all technical and scientific terms used herein have the same meaning as commonly understood by one having ordinary skill in the art to which the disclosure pertains.

All publications, patents and patent applications cited herein, whether supra or infra, are hereby incorporated by reference in their entirety.

Glomerular ultrafiltration is located within the renal glomerulus, a combination of blood vessels and cells. Highly specialized podocyte cells perform the filtering work. These cells are the target of injury in many glomerular diseases affecting the shape of foot processes. The apoptosis of the podocytes (which results in a reduction of podocyte number and their density) is involved in a variety of nephrotic syndromes. For example, the reduction of number and density of the podocytes can results in kidney disorders, including but not limited to proteinuric disorders such as Diabetic nephropathy, Nephrotic syndromes (i.e. intrinsic renal failure), Nephritic syndromes, Toxic lesions of kidneys, Glomerular diseases, such as membranous glomerulonephritis, Focal segmental glomerulosclerosis (FSGS), IgA nephropathy (i.e., Berger's disease), IgM nephropathy, Membranoproliferative glomerulonephritis, Membranous nephropathy, Minimal change disease, Hypertensive nephrosclerosis and Interstitial nephritis proteinuria, glomerulosclerosis and renal dysfunction.

### Definitions

As used in this specification and the appended claims, the singular forms “a, “an” and “the” include plural referents unless the content clearly dictates otherwise.

The term “peptide,” “polypeptide” and “protein” are used interchangeably to denote a sequence polymer of at least two amino acids covalently linked by an amide bond (also referred herein as peptide bond).

As used herein the term “target molecule” refers to any molecule that is associated with a tissue (e.g. “at risk”, diseased or damaged tissue). A “target cell” is meant to be a cell to which a protein or targeting domain thereof can specifically bind.

“Binding” or “specific binding” are used interchangeably herein and indicates that a protein (or the targeting polypeptide domain thereof or the activator domain thereof) exhibits substantial affinity for a specific molecule (e.g., targeting domain exhibits substantial affinity for a target molecule, or an activator domain exhibits substantial affinity for a molecule associated with the surface of a cell such as a growth factor receptor) or a cell or tissue bearing the molecule and is said to occur when the protein (or the targeting polypeptide domain thereof or the activator domain thereof) has a substantial affinity for a specific molecule and is selective in that it does not exhibit significant cross-reactivity with other molecules.

The term “targeting moiety”, “targeting domain,” “targeting polypeptide” or “targeting module” are used herein interchangeably and refer to molecules that selectively localize the chimeric protein in a particular tissue or region of the body. The localization can be mediated by specific recognition of molecular determinants, molecular size of the targeting domain, ionic interactions, hydrophobic interactions and the like.

As used herein, the terms “therapeutic moiety,” “activator domain,” “activator polypeptide”, “signaling arm” and “effector module” are used herein interchangeably and refers to any agents useful for therapy and that are non-toxic, do not have a cytotoxic effect or are not detrimental to the cells. Such agents can include, but not limited to, growth factors.

As used herein, term “antibody” includes but is not limited to: (i) a Fab fragment, a monovalent fragment consisting of the VL, VH, CL and CH1 domains; (ii) F(ab)2 and F(ab′)2 fragments, a bivalent fragment comprising two Fab fragments linked by a disulfide bridge at the hinge region; (iii) a Fd fragment consisting of the VH and CH1 domains; (iv) a scFv fragment consisting of the V L and VI-H domains of a single ann of an antibody, (v) a dAb fragment which consists of a VII domain; and (vi) an isolated complementarity determining region (CDR).

The term “antibody fragment” of an antibody as used herein refers to one or more portions of an antibody. In some embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv). The term “single-chain Fv” or “scFv” antibody refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen-binding. For a review of scFv, see Pluckthun (1994) THE Pharmacology Of Monoclonal Antibodies, vol. 113, Rosenburg and Moore eds. Springer-Verlag, New York, pp. 269-315.

The term “specifically (or selectively) binds” of an antibody fragment to an antigen or an epitope refers to a binding reaction that is determinative of the presence of a cognate antigen or an epitope in a heterogeneous population of proteins and other biologics. Typically the antibody fragment binds to the target antigen with an affinity that is at least two-fold greater than its affinity for binding to a non-specific antigen and with a dissociation rate constant (KD) (koff/kon) is of less than less than 10⁻² M, less than 10⁻³ M, less than 10⁻⁴ M, less than 10⁻⁵ M, less than 10⁻⁶ M, less than 10⁻⁷ M, less than 10⁻⁸ M, or less than 10⁻⁹ M or about 10⁻² M, about 10⁻³ M, about 10⁻⁴ M, less than 10⁻⁵ M, about 10⁻⁶ M, about 10⁻⁷ M, about 10⁻⁸ M, about 10⁻⁹ M, about 10−¹⁰ M or ranging from 10⁻² M to 10⁻³ M, 10⁻³ M to 10⁻⁴ M, 10⁻⁴ M to 10⁻⁵ M, 10⁻⁵ M to 10⁻⁶ M, 10⁻⁶ M to 10⁻⁷ M, 10⁻⁷ M to 10⁻⁸ M, 10⁻⁸ M to 10⁻⁹ M, 10⁻⁹ M to 10⁻¹⁰ M.

“Identity,” as known in the art, is a relationship between two or more polypeptide or protein sequences, as determined by comparing the sequences. In the art, “identity” also refers to the degree of sequence relatedness between polypeptides or proteins, as determined by the match between strings of such sequences. “Identity” can be readily calculated by any bioinformational methods known in the art.

The term “parent polypeptide” refers to a wild-type polypeptide and the amino acid sequence or nucleotide sequence of the wild-type polypeptide is part of a publicly accessible protein database (e.g., EMBL Nucleotide Sequence Database, NCBI Entrez, ExPasy, Protein Data Bank, Swiss-Prot, UniProt Knowledgebase, and the like).

The term “mutant polypeptide” or “polypeptide variant” refers to a form of a polypeptide, wherein its amino acid sequence differs from the amino acid sequence of its corresponding wild-type (parent) form, naturally existing form or any other parent form. A mutant polypeptide can contain one or more mutations, e.g., substitution, insertion, deletion, addition etc. which result in the mutant polypeptide. Generally, variants are overall closely similar, and, in many regions, identical to the reference polypeptide. As used herein, “variant” refers to a polypeptide, differing in sequence from a native protein but retaining at least one functional and/or therapeutic property thereof as described elsewhere herein or otherwise known in the art.

The term “corresponding to a parent polypeptide” is used to describe a polypeptide of the disclosure, wherein the amino acid sequence of the polypeptide differs from the amino acid sequence of the corresponding parent polypeptide only by the presence of at least one amino acid variation. For example, the amino acid sequence of the variant polypeptide differs from the amino acid sequence of the corresponding parent polypeptide by the presence one, two, three, four, five, six, seven, eight, none ten or more amino acid variation. Typically, the amino acid sequences of the variant polypeptide and the parent polypeptide exhibit a high percentage of identity. In one example, “corresponding to a parent polypeptide” means that the amino acid sequence of the variant polypeptide has at least about 50% identity, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98% identity or at least about 99% identity to the amino acid sequence of the parent polypeptide. In another example, the nucleic acid sequence that encodes the variant polypeptide has at least about 50% identity, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98% identity or at least about 99% identity to the nucleic acid sequence encoding the parent polypeptide.

The term “substantial identity” or “substantial similarity,” as used herein, when referring to a nucleic acid or fragment thereof, indicates that when optimally aligned with appropriate nucleotide insertions or deletions with another nucleic acid (or its complementary strand), there is nucleotide sequence identity in at least about 80%, 81%, 82%, 83%, 84%, 85%, 86%, 87%, 88%, 89%, 90%, 91%, 92%, 93%, 94%, 95%, 96%, 97%, 98%, 99% of the sequence. The term “substantial identity” or “substantial similarity,” as used herein, when referring to a protein or fragment thereof, indicates that when optimally aligned there is an amino acid sequence identity in at least about 95% to 99% of the sequence. Two sequences are “substantially identical” if two sequences have a specified percentage of amino acid residues or nucleotides that are the same (e.g., 90% identity, optionally 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or, in the case of a peptide or polypeptide, at least about 10 amino acids) in length, or in some cases over a region that is 100 to 500 or 1000 or more nucleotides (or 20, 50, 200 or more amino acids) in length. Two examples of algorithms that are suitable for determining percent sequence identity and sequence similarity are the BLAST and BLAST 2.0 algorithms, which are described in Altschul et al., 1977, Nuc. Acids Res. 25:3389-3402; and Altschul et al., 1990, J. Mol. Biol. 215:403-410, respectively. Software for performing BLAST analyses is publicly available through the National Center for Biotechnology Information.

The terms “mutation” and “modification” in the context of a polypeptide as used herein can include substitution, addition or deletion of one or more amino acids.

The term “damaged cell” or “damaged tissue,” as used herein, means and includes biological cell or tissue that is damaged. In some embodiments, the damaged cells comprise apoptotic cells such. In some embodiments, the damaged cells comprise podocyte cells. In some embodiments, the damaged cells comprise glomerular mesangial cells. In some embodiments, the damaged cells comprise parietal epithelial cells that form Bowman's capsule. In some embodiments, the damaged cells comprise glycocalyx-coated fenestrated endothelial cells.

The term “therapeutically effective amount,” as used herein, means the amount of the protein or agent that will elicit the biological or medical response of a tissue, system, animal or human that is being sought by the researcher, veterinarian, medical doctor or other clinician.

The term “pharmaceutically acceptable,” as used herein, means the carrier, diluent or excipient must be compatible with the other ingredients of the formulation and not deleterious to the recipient thereof.

The terms “approximately” or “about” in reference to a number generally include numbers that fall within +/−20%, in some embodiments, +/−10%, in some embodiments, +/−5%, in some embodiments +/−1%, in some embodiments +/−0.5% of the number unless otherwise stated or otherwise evident from the context (except where such number would impermissibly exceed 100% of a possible value).

“Linked”, as used herein with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In some embodiments, the linkage is a covalent linkage. In other embodiments, the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linking moiety” or “linking portion”, the linkage between the two linked moieties is indirect, and typically each of the linked moieties is covalently bonded to the linking moiety. Two moieties may be linked using a “linker”. A linker can be any suitable moiety that reacts with the entities to be linked within a reasonable period of time, under conditions consistent with stability of the entities (portions of which may be protected as appropriate, depending upon the conditions), and in sufficient amount, to produce a reasonable yield. It will be appreciated that after the linker has reacted with the entities to be linked, the term “linker” may refer to the part of the resulting structure that originated from the linker. A linking moiety may comprise a portion whose main purpose may be to spatially separate the entities from each other.

A “subject” treated according to the disclosure is typically a human, a non-human primate, or a lower animal (e.g., a mouse or rat). In some embodiments the subject is an adult, e.g., a human at least 18 years of age. In some embodiments, a human subject is at least 12 years of age. In some embodiments the subject is a child, e.g., a human between 0 and 4 years of age, or between 5 and 11 years of age.

### Chimeric Proteins

Chimeric proteins (also referred herein as fusion proteins or bi-specific fusion proteins) provided herein are capable of specific binding to two or more different specific molecules. In some embodiments, the chimeric protein comprises a targeting domain having a binding specificity to a first specific target molecule, an activator domain having a binding specificity to a second target molecule. In some embodiments, the chimeric protein further comprises a peptide scaffold. In some embodiments, the peptide scaffold comprises a half-life modulator.

In some embodiments, the chimeric protein comprises a targeting domain having a binding specificity to a first specific target molecule, an activator domain having a binding specificity to a growth factor receptor. In some embodiments, the chimeric protein comprises a targeting domain having a binding specificity to a first specific target molecule, an activator domain having a binding specificity to a second target molecule, and a half-life modulator. In some embodiments, the chimeric protein comprises a targeting domain having a binding specificity to a first specific target molecule, an activator domain having a binding specificity to a growth factor receptor, and a half-life modulator. In some embodiments, the half-life modulator extends the half-life of the chimeric protein. In some embodiments, the chimeric protein comprises a targeting domain having a binding specificity to a first specific target molecule, an activator domain having a binding specificity to a second specific target molecule, a peptide and at least one linker. In some embodiments, the peptide comprises or consists of a half-life modulator that extends the half-life of the chimeric protein. In some embodiments, the activator domain is at the N-terminus of the chimeric protein. In some embodiments, the targeting domain is at the C-terminus of the chimeric protein. In some embodiments, the activator domain is at the N-terminus of the chimeric protein and the targeting domain is at the C-terminus of the chimeric protein.

Yet in other embodiments, the activator domain is at the C-terminus of the chimeric protein. In some embodiments, the targeting domain is at the N-terminus of the chimeric protein. In some embodiments, the activator domain is at the C-terminus of the chimeric protein and the targeting domain is at the N-terminus of the chimeric protein.

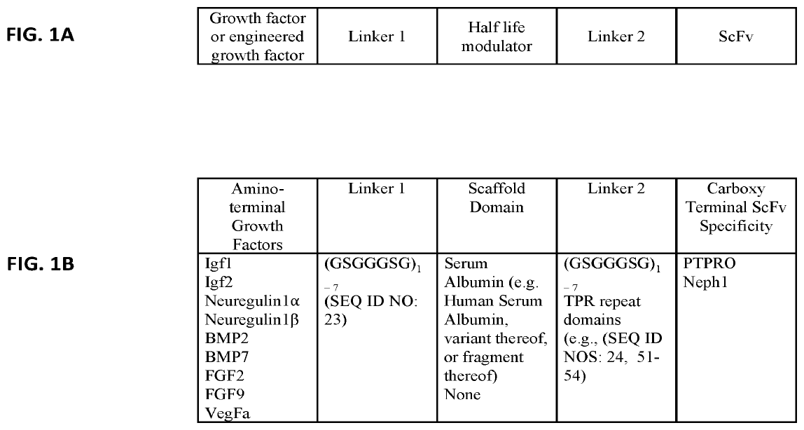

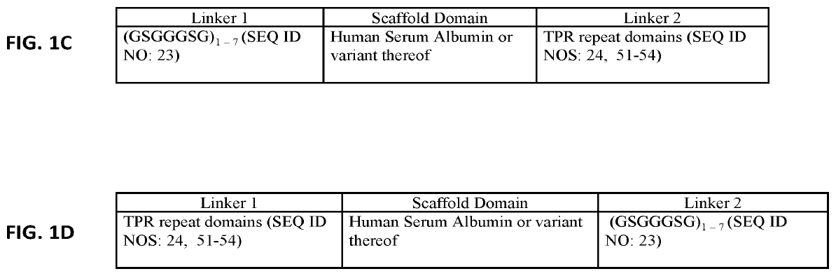

In some embodiments, the chimeric protein further comprise at least one linker. In some embodiments, the chimeric protein further comprises at least two linkers. In some embodiments, the chimeric protein further comprises two linkers. In some embodiments, a first linker links the activator domain to the peptide (or half-life modulator). In some embodiments, a second linker links the targeting domain to the peptide (or half-life modulator). (see FIG. TA and FIG. 1B)

In some embodiments, the activator domain binds to a growth factor receptor on a cell surface. The binding of the activator domain to its receptor is intended to modulate a specific biological effect, such as, activate the intracellular signaling pathway associated with cell survival. In some embodiments, binding of the activator domain to its receptor is intended to positively regulate survival of the targeted cells or tissue. In particular, the activator domain of the chimeric protein can promote survival signaling. In particular, the activator domain of the chimeric protein can promote apoptosis escape. In some embodiments, the activator domain comprises or consists of a growth factor of fragment thereof. In some embodiments, the growth factor is engineered to reduce potency while retaining the ability to activate the cognate growth factor receptor. In some embodiments, wild type growth factors or fragment thereof can be used as activator domains. In some embodiments, modified growth factors (also referred herein as growth factor variants) or fragment thereof can be used as activator domains. In some embodiments, the modified growth factor is engineered to reduce potency while retaining the ability to activate the cognate growth factor receptor.

In some embodiments, the chimeric protein comprises an activator domain having a growth factor variant that is engineered to give the chimeric protein at least an order of magnitude lower EC50 in damaged tissue than in healthy tissue. For example, the chimeric protein domain comprises a growth factor variant and has an EC50 in damaged tissue that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower than the EC50 in healthy tissue.

In some embodiments, the activator domain comprises or consists of growth factor (or variant thereof or fragment thereof). In some embodiments, the targeting domain is a protein (or variant thereof or fragment thereof) having a binding affinity to a protein sequence specifically expressed and displayed at the cell surface in the glomerulus.

According to some aspects of the disclosure, the activator domain comprises or consists of growth factor (or variant thereof or fragment thereof) and the targeting domain comprises or consists of a protein (or variant thereof or fragment thereof) having a binding affinity to a protein sequence specifically expressed and displayed at the cell surface in the glomerulus.

According to some aspects of the disclosure, the chimeric protein comprises a growth factor and a single-chain variable fragment having high affinity and selectivity for a protein sequence specifically expressed and displayed at the cell surface in the glomerulus. In some embodiments, the chimeric protein comprises a growth factor and a single-chain variable fragment with high affinity and selectivity for protein tyrosine phosphatase receptor type O (PTPRO, also known as glomerular epithelial protein-1GLEPP1).

### Targeting Domain

The targeting domain is generally used to target the chimeric proteins to a target cell of a tissue. In some embodiments, the target cell is undergoing apoptosis. In some embodiments, the tissue is kidney tissue. In some embodiments, the tissue is the glomerular visceral epithelium in the kidney. In some embodiments, the tissue is the simple squamous epithelium of the renal corpuscle.

In some embodiments, the targeting domain has a specific binding affinity to a target molecule associated with a tissue (for example, to a protein part of a publicly accessible protein database Protein Atlas). In some embodiments, binding of the targeting domain to the target molecule does not have or does not modulate a biological activity. As used herein, “biological activity” refers to a defined, known activity performed by exposure of a molecule to a domain of the protein.

In some embodiments, the targeting domain comprises one or more antibody binding fragment. In some embodiments, the targeting domain comprises one or more antibody variable regions (e.g., scFv). In some embodiments, the targeting domain can comprise one or more of a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment.

Single chain Fv or “scFv” antibody fragments comprise the VH and VL domains of an antibody in a single polypeptide chain, are capable of being expressed as a single chain polypeptide, and retain the specificity of the intact antibody from which it is derived. Generally, the scFv polypeptide further comprises a polypeptide linker between the VH and VL domain that enables the scFv to form the desired structure for target binding. Any linkers suitable for connecting the VH and VL chains of an scFV can be used.

As used herein an scFv may have the VL and VH variable regions in either order, e.g., with respect to the N-terminal and C-terminal ends of the polypeptide, the scFv may comprise VL-linker-VH or may comprise VH-linker-VL.

Such antibodies or antibody fragments may be produced from intact antibodies using methods known in the art, or may be produced recombinantly, using standard recombinant DNA and protein expression technologies, or may be produced by grafting of complementarity determining regions to stable scFv frameworks.

In some embodiments, the chimeric protein (or its targeting domain) binds to the target molecule with a K_{d} of less than 10⁻⁶ M, less than 10⁻⁷ M, less than 10⁻⁸ M, less than 10⁻⁹ M or less than 10⁻¹ M. In some embodiments, the chimeric protein (or its targeting domain) binds to the target molecule with a K_{d} of about 10⁻⁶ M, about 10⁻⁷ M, about 10⁻⁸ M, about 10⁻⁹ M or about 10⁻¹ M. In some embodiments, the chimeric protein (or its targeting domain) binds to the target molecule with a K_{d} of from about 10⁻⁶ M to about 10⁻⁷ M, from about 10⁻⁷ M to about 10⁻⁸ M, from about 10⁻⁸ M to about 10⁻⁹ M, from about 10⁻⁹ M to about 10⁻¹⁰ M.

In some embodiments, the targeting domain comprises an antibody fragment (e.g. scFv) having a binding specificity to a target protein or antigen displayed the cell surface in the glomerulus. In some embodiments, the targeting domain targets podocytes, which are cells of the glomerular visceral epithelium in the kidney. In some embodiments, the targeting domain targets parietal epithelial cells that form Bowman's capsule. In some embodiments, the targeting domain targets glycocalyx-coated fenestrated endothelial cells.

In some embodiments, the target protein is Protein Tyrosine Phosphatase Receptor Type O (PTPRO), Podocin, Phospholipase A2 Receptor 1, Integrin Subunit alpha 8, Apoptosis resistant E3 ubiquitin protein ligase 1, Integrin Subunit alpha 8, Apoptosis resistant E3 ubiquitin protein ligase 1, Podocalyxin, Synaptopodin, Alpha 3 integrin, Cysteine rich transmembrane BMP regulator 1, FAT atypical cadherin 1, NEPH1, Dystroglycan, or Podoplanin.

In some embodiments, the target protein is Protein Tyrosine Phosphatase Receptor Type O (PTPRO) and the targeting domain comprises or consists of a scFv having a specific binding affinity to PTPRO (See FIG. 1B).

### Protein Tyrosine Phosphatase Receptor Type O (PTPRO)

The protein tyrosine phosphatase receptor type O (PTPRO, also referred as GLEPP1) is a type of phosphotyrosine protein phosphatase (PTP) receptor that was first identified and cloned in the human glomerulus. PTPRO is a transmembrane protein, and its intracellular domain contains the Protein Tyrosine Phosphatase domain that catalyzes the dephosphorylation of tyrosine residues. GLEPP1 is located on the apical cell membrane of visceral glomerular epithelial cell and foot processes, has been used as a marker of acute podocyte injury. GLEPP1 is also found in podocytes.

These proteins play a role in regulating the glomerular pressure/filtration rate relationship through an effect on podocyte structure and function.

In some embodiments, the scFvs directed against PTPRO may modulate albumin permeability or augment intrinsic phosphatase activity. In other embodiments, the scFvs directed against PTPRO have no effect on the biological function of PTPRO.

### Podocin

Podocin is a member of the stomatin family. It is uniquely expressed in the kidney. It is a membrane protein with an hairpin-like structure with two cytoplasmic ends at the N- and C-terminus. The protein is localized on the membranes of the podocyte's pedicels.

In some embodiments, the scFvs directed against podocin modulate the biological function of podocin. In other embodiments, the scFvs directed against podocin have no effect on the biological function of podocin.

### Phospholipase A2 Receptor] (PLA2R1)

Phospholipase A2 Receptor 1 (also referred as Phospholipase A2 Receptor) is a type I transmembrane receptor. It is a 185-kDa protein expressed on the surface of human podocytes. M-type phospholipase A2 receptor is the major antigen in idiopathic membranous nephropathy.

In some embodiments, the scFvs directed against Phospholipase A2 Receptor modulate the biological function of Phospholipase A2 Receptor. In other embodiments, the scFvs directed against Phospholipase A2 Receptor does not modulate the biological function of Phospholipase A2 Receptor.

### Integrin Subunit Alpha 8 (Itga8)

Integrin alpha-8/beta 1 (ITGA8) functions in the genesis of kidney by regulating the recruitment of mesenchymal cells into epithelial structures. It recognizes the sequence R-G-D in a wide array of ligands including TNC, FN1, SPP1 TGFB1, TGFB3 and VTN. ITGA8 has been shown to accumulate in the renal glomeruli in response to renal injury, such as diabetic nephropathy.

Itga8 is an integrin chain which is predominantly expressed on mesenchymal cells of the glomerulus (e.g. mesangial cells). It confers mechanical stability of the glomerular capillary tuft. Itga8 may have a role in healing glomerulonephritis by reducing the proliferation of mesangial cells and protect cells from apoptosis.

In some embodiments, the scFvs directed against Itga8 modulate the biological function of Itga8. In other embodiments, the scFvs directed against Itga8 does not modulate the biological function of Itga8.

### Apoptosis Resistant E3 Ubiquitin Protein Ligase 1 (ARELI)

Apoptosis resistant E3 ubiquitin protein ligase 1 (AREL1) inhibits apoptosis by ubiquitinating and targeting for degradation a number of proapoptotic proteins including DIABLO/SMAC, HTRA2 and SEPT4/ARTS which are released from the mitochondrion into the cytosol following apoptotic stimulation. AREL1 is expressed at the cell membrane in renal glomeruli.

In some embodiments, the scFvs directed against AREL1 modulate the biological function of ARELL. In other embodiments, the scFvs directed against AREL1 does not modulate the biological function of AREL1.

### Podocalyxin

Podocalyxin is a CD34-related sialomucin protein that is highly-expressed by podocytes. Podocalyxin was initially identified in kidney glomeruli, where it is abundant. It coats the secondary foot processes of the podocytes.

Podocalyxin is expressed on the apical membrane of the podocyte. It is involved in the regulation of both adhesion and cell morphology and cancer progression. It functions as an anti-adhesive molecule that can maintain an open filtration pathway between neighboring foot processes in the podocyte by charge repulsion, thereby keeping the urinary filtration barrier open. It acts as a pro-adhesive molecule, enhancing the adherence of cells to immobilized ligands, increasing the rate of migration and cell-cell contacts in an integrin-dependent manner. The protein induces the formation of apical actin-dependent microvilli. It is involved in the formation of a preapical plasma membrane subdomain to set up initial epithelial polarization and the apical lumen formation during renal tubulogenesis. It plays a role in cancer development and aggressiveness by inducing cell migration and invasion through its interaction with the actin-binding protein EZR. It affects EZR-dependent signaling events, leading to increased activities of the MAPK and PI3K pathways in cancer cells.

In some embodiments, the scFvs directed against podocalyxin modulate the biological function of podocalyxin. In other embodiments, the scFvs directed against podocalyxin does not modulate the biological function of podocalyxin.

### Synaptopodin

Synaptopodin is a podocyte actin-binding protein that regulates the cytoskeleton. Synaptopodin is essential for the integrity of the podocyte actin cytoskeleton and for the regulation of podocyte cell migration. Synaptopodin synchronizes podocyte actin dynamics and cell migration by blocking Smurf-1-mediated ubiquitination of RhoA.

In some embodiments, the scFvs directed against synaptopodin modulate the biological function of synaptopodin. In other embodiments, the scFvs directed against synaptopodin does not modulate the biological function of synaptopodin.

### Alpha 3 Integrin

Integrins are adhesion receptors that link the extracellular matrix to the cell cytoskeleton. Integrins are composed of two subunits, a large alpha chain and a smaller beta chain. Integrins are ubiquitously localized in the kidney. Integrin alpha3beta1 is essential for podocyte development and function, and for assembly of the glomerular basement membrane.

In some embodiments, the scFvs directed against Alpha 3 integrin modulate the biological function of Alpha 3 integrin. In other embodiments, the scFvs directed against Alpha 3 integrin does not modulate the biological function of Alpha 3 integrin.

### Cysteine Rich Transmembrane BMP Regulator] (CRIM1)

Cysteine rich transmembrane BMP regulator 1 (CRIM1) has tissue enrichment expression in the renal glomeruli and is thought to play a role in tissue development through interactions with members of the transforming growth factor beta family, such as bone morphogenetic proteins.

In some embodiments, the scFvs directed against CRIM1 modulate the biological function of CRIM1. In other embodiments, the scFvs directed against CRIM1 does not modulate the biological function of CRIM1.

### FAT Atypical Cadherin 1 (FAT1)

FAT1 is an essential protein for cellular polarization, directed cell migration and modulating cell-cell contact and expressed in the highly polarized podocyte cell-type.

In some embodiments, the scFvs directed against FAT1 modulate the biological function of FAT1. In other embodiments, the scFvs directed against FAT1 does not modulate the biological function of FAT1.

### NEPH1

Kin of IRRE-like protein 1, also known as NEPH1, is a protein that in humans is encoded by the KIRREL gene. NEPH1 is a podocyte transmembrane protein of the Ig superfamily. The cytoplasmic domains of these proteins interact with the C terminus of podocin. It is expressed in kidney podocytes, cells involved in ensuring size- and charge-selective ultrafiltration. It is crucial podocyte molecule in the kidney glomerular filtration barrier. It is a major component of the podocyte slit diaphragm and is essential for maintaining normal glomerular permeability.

In some embodiments, the scFvs directed against NEPH1 modulate the biological function of NEPH1. In other embodiments, the scFvs directed against NEPH1 does not modulate the biological function of NEPH1.

### Dystroglycan (DG)

In kidney, dystroglycan (DG) has been shown to cover the basolateral and apical membranes of the podocyte. alpha-DG is heavily glycosylated, which is important for its binding to laminin and agrin in the glomerular basement membrane. Alpha-DG covers the whole podocyte cell membrane in the rat, and is expressed at both the basolateral and apical sides of the podocyte. This localization suggests that alpha-DG plays a dual role in the maintenance of the unique architecture of podocytes by its binding to the glomerular basement membrane, and in the maintenance of the integrity of the filtration slit, respectively. Dystroglycan was diffusely found over the entire cell surface of the podocytes.

In some embodiments, the scFvs directed against DG modulate the biological function of DG. In other embodiments, the scFvs directed against DG does not modulate the biological function of DG.

### Podoplanin

Podoplanin is a glomerular podocyte membrane mucoprotein. Podoplanin plays a role in maintaining the unique shape of podocyte foot processes and glomerular permeability. In rats, the 43-kD integral membrane protein podoplanin is localized on the surface of podocytes, and transcriptionally downregulated puromycin nephrosis.

In some embodiments, the scFvs directed against podoplanin modulate the biological function of podoplanin. In other embodiments, the scFvs directed against podoplanin does not modulate the biological function of podoplanin.

### Activator Domain

The activator domain can be any polypeptide that detectably modulates the activity of a cellular network. In some embodiments, the activator domain is capable of activating signal transduction pathways by binding to a receptor at the surface a cell. In some embodiments, certain activator domains are growth factor polypeptides, or any agonist of the receptor. It will be apparent that such modulation may be an increase in the activity of the cellular network such as induction of proliferation of cells, induction of cell growth, promotion of cell survival and/or inhibition of apoptosis and/or escape from apoptosis.

In some embodiments, the activator domain comprises a change in the amino acid sequence, the three-dimensional structure of the protein, and/or the activity of the protein, relative to the wild-type form of the protein.

In some embodiments, the activator domain comprises or consists of a growth factor or fragment thereof or variant thereof, the growth factor being selected from the group consisting of. IGF-1, IGF-2, Neuregulin1α, Neuregulin1β, BMP2, FGF2, FGF9 and VEGF-A.

In some embodiments, the growth factor fragments can have a length of about or greater than about 25 amino acid long.

In some embodiments, the activator domain comprises or consists of a growth factor having amino acid sequence modification relative to the wild-type growth factor (e.g. IGF-1) to decrease its binding to its natural receptor (e.g. IGF-1 receptor), to decrease its binding to binding proteins (e.g. IGF binding proteins) and/or decrease its activation of its natural receptor (e.g. IGF-1 receptor). In some embodiments, the activator domain is a growth factor having amino acid sequence modification that reduce (e.g., for about 1-5%, 5-10%, 10%-20%, about 20%-40%, about 50%, about 40%-60%, about 60%-80%, about 80%-90%, 90-95%) the binding to its natural receptor (e.g. IGF-1 receptor).

A growth factor polypeptide detectably modulates activation of a growth factor receptor. In some embodiments, the activator domain of the chimeric protein is a growth factor, variant or fragment thereof that retains at least about 0.01% of wild-type biological activity. In some embodiments, the activator domain of the chimeric protein is a growth factor, variant or fragment thereof that retain at least about 0.1%, at least about 1%, at least about 10%, of wild-type biological activity. In some embodiments, the activator domain of the chimeric protein is a growth factor, variant or fragment thereof that retains between about 0.01% to about 0.1% of wild-type biological activity. In some embodiments, the activator domain of the chimeric protein is a growth factor, variant or fragment thereof that retains between about 0.01% to about 1% of wild-type biological activity. In some embodiments, the activator domain of the chimeric protein is a growth factor, variant or fragment thereof that retains between about 0.01% to about 10% of wild-type biological activity. In some embodiments, the activator domain of the chimeric protein is a growth factor, variant or fragment thereof that retains between about 0.1% to about 1% of wild-type biological activity. In some embodiments, the activator domain of the chimeric protein is a growth factor, variant or fragment thereof that retains between about 0.10% to about 10% of wild-type biological activity. In some embodiments, the activator domain of the chimeric protein is a growth factor, variant or fragment thereof that retains between about 01% to about 10% of wild-type biological activity. Biological activity in some embodiments can be determined by measuring activation of the corresponding growth factor receptor in appropriate cells. In some embodiments, activation may be assessed, for example, by measuring phosphorylation of receptor kinase or downstream effector proteins, such as, but not limited to, AKT, S6, ERK, JNK, mTOR, etc.

In some embodiments, the chimeric proteins provided herein having such variant growth factors have a higher specificity to the damaged tissue targeted or apoptotic cells.

### Insulin-Like Growth Factors (IGFs) and Derivatives Thereof

The insulin-like growth factors (IGFs) constitute a family of proteins having insulin-like and growth stimulating properties. Insulin-like growth factors (IGF-1 and IGF-2) are related structurally to proinsulin and promote cell proliferation, differentiation, and survival, as well as insulin-like metabolic effects, in most cell types and tissues.

The IGFs Human IGF-1 is a 70 amino acids basic peptide having the protein shown in SEQ ID NO: 1, respectively. IGF-1 and extracellular tyrosine kinase receptor (e.g. IGF-1 receptor) are important for cellular processes such as cell proliferation and survival. Binding of IGF-1 or variant thereof to the IGF-1 receptor stimulates kinase activity, leading to phosphorylation of multiple substrates, thereby initiating signaling cascades. The chimeric proteins disclosed herein can maintain the ability to signal through the extracellular receptor, for example IGF-1 receptor. The activator domain IGF-1 stimulates cell proliferation and survival through activation of the AKT pathway. Upon binding of IGF-I to the IGF-1 receptor, a tyrosine kinase, phosphorylates tyrosine residues on two major substrates, IRS-1 and Shc, which subsequently signal through the Ras/Raf and PI 3-kinase/AKT pathways.

The interaction of IGF-1 (and IGF-2) with the IGF-1 receptor is regulated by IGF binding Proteins (IGFBPs). All six IGFBPs (particularly IGFBP5) have been shown to inhibit IGF action, but in some instances a stimulatory effect has been observed. At least 99% of the IGF in the circulation is normally bound to IGFBPs.

In some embodiments, the activator domain is a variant of the human IGF-1 or fragment thereof. In some embodiments, the variant of IGF-1 or fragment thereof is capable of maintaining selectivity to the IGF-1 receptor. FIG. 7 shows residues identified via application of an algorithm that considers solvent accessibility, hydrophobicity, and temperature factor. Collectively, these residues represent the hydrophobic core of IGF-1. Hydrophobic core positions of IGF-1 are depicted as spheres (pdb model 1H02) at FIG. 7.

In some embodiments, the IGF-1 variant is modified to reduce binding to IGF-1 binding proteins (IGFBPs) relative to wild-type IGF-1 while maintaining its ability to activate the AKT pathway. In some embodiments, the IGF-1 variant can activate the IGF-1 receptor with a decreased potency for non-target cells, as assessed by pAKT EC50. EC50 is defined as the concentration needed to achieve the half maximal level of pAKT signaling.

In some embodiments, the IGF-1 variant comprises a substitution at one or more of the tyrosine residues. In some embodiments, the IGF-1 variant comprises one or more substitutions at position Y24, Y31 and Y60.

In some embodiments, the IGF-1 variant can comprise a single tyrosine substitution at position Y31, or Y24, or Y60. In some embodiments, the IGF-1 variant can comprise a tyrosine substitution at positions Y24 and Y31, Y24 and Y60, Y31 and Y60, or Y24 and Y60. In some embodiments, the IGF-1 variant can comprise a tyrosine substitution at position Y24, Y31 and Y60.

In some embodiments, the tyrosine at position 24 can be substituted with alanine (Y24A), valine (Y24V), leucine (Y24L), glycine (Y24G), methionine (Y24M), serine (Y24S), asparagine (Y24N) or Glutamine (Y24Q). In some embodiments, the tyrosine at position 31 can be substituted with alanine (Y31A), valine (Y31V), leucine (Y31L), glycine (Y31G), methionine (Y31M), serine (Y31S), asparagine (Y31N) or Glutamine (Y31Q). In some embodiments, the tyrosine at position 60 can be substituted with alanine (Y60A), valine (Y60V), leucine (Y60L), glycine (Y60G), methionine (Y60M), serine (Y60S), asparagine (Y60N) or Glutamine (Y60Q).

In another exemplary embodiment, the IGF-1 variant can comprise one or more of the following substitutions, Y24L, Y31A, and Y60L relative to wild type IGF-1. For example, the IGF-1 variant can comprise the Y24L substitution and the Y31A substitution or the IGF-1 variant can comprise the Y24L substitution, the Y31A substitution and the Y60L. In some embodiments, one or more tyrosine residues (Y24, Y31, Y60 or combinations thereof) can be substituted for a short aliphatic amino acid. In some embodiments, one or more tyrosine residues (Y24, Y31, Y60 or combinations thereof) can be substituted for a polar amino acid.

In some embodiments, one or more tyrosine residues (Y24, Y31, Y60 or combinations thereof) can be substituted for leucine, alanine, isoleucine, serine, threonine or any other amino acid. In some embodiments, the glutamic acid at position 3 can be substituted with alanine, arginine, asparagine, cysteine, glutamine, glycine, histidine, isoleucine, leucine, lysine, methionine, phenylalanine, proline, serine, threonine, tryptophan, tyrosine or valine. In some embodiments, the glutamic acid at position 3 is replaced with a basic and/or aliphatic amino acid. In some embodiments, the glutamic acid at position 3 can be substituted with arginine (E3R) or lysine (E3K). In some embodiments, the IGF-1 variant comprises E3R substitution.

In some embodiments, the IGF-1 variant comprises a substitution at the position 3 and 31. For example, the IGF-1 variant can comprise E3R and Y31A substitutions or E3K and Y31A substitutions. In some embodiments, the activator domain comprises or consists of an amino acid sequence having SEQ ID NO: 2 or SEQ ID NO: 64. In some embodiments, the IGF-1 variant comprises a substitution at the position E3 and Y24. For example, the IGF-1 variant comprises E3R and Y24L substitutions. In some embodiments, the activator domain comprises or consists of an amino acid sequence having SEQ ID NO: 25 or SEQ ID NO: 63. In some embodiments, the IGF-1 variant comprises a substitution at the position E3 and Y60. For example, the IGF-1 variant comprises E3R and Y60L substitutions. In some embodiments, the activator domain comprises or consists of an amino acid sequence having SEQ ID NO: 26 or SEQ ID NO: 65. In some embodiments, the IGF-1 variant comprises a substitution at the position E3, Y24 and Y31. For example, the IGF-1 variant comprises E3R or E3K, Y24L, Y31A substitutions. In some embodiments, the activator domain comprises or consists of an amino acid sequence having SEQ ID NO: 27. In some embodiments, the IGF-1 variant comprises a substitution at the position E3, Y24 and Y60. For example, the IGF-1 variant comprises E3R or E3K, Y24L and Y60L substitutions. In some embodiments, the activator domain comprises or consists of an amino acid sequence having SEQ ID NO: 28. In some embodiments, the IGF-1 variant comprises a substitution at the position E3, Y31 and Y60. For example, the IGF-1 variant comprises E3R or E3K, Y31A and Y60L substitutions. In some embodiments, the activator domain comprises or consists of an amino acid sequence having SEQ ID NO: 29. In some embodiments, the IGF-1 variant comprises a substitution at the position E3, Y24, Y31 and Y60. For example, the IGF-1 variant comprises E3R or E3K, Y24L, Y31A and Y60L substitutions. In some embodiments, the activator domain comprises or consists of an amino acid sequence having SEQ ID NO: 30.

In some embodiments, the activator domain is a derivative of the human IGF-1 comprising one or more of the following modifications: a N-terminal 13-residue extension (IGF-1 LONG), a deletion of amino acids 1-3 (Des-1-3), a substitution replacing Arg for a Glu at the 3 position of the polypeptide (E3R), no Arginine at position 37 (R37X), a deletion of amino acids 68-70 (3X), an N-terminal 13-residue extension and a substitution replacing Arg for a Glu at the 3 position of the wild-type polypeptide (LR3), substitutions of one or more of tyrosine residues Y24, Y31, Y60 or combinations thereof (e.g. Y24L, Y31A, Y60L substitutions or combinations thereof).

In some embodiments, the activator domain is variant of the human IGF-1 comprising a mutation (e.g. substitution, deletion) at one or more residues at position 24 to 60 of wild-type human IGF-1 (SEQ ID NO. 1).

In some embodiments, the activator domain is a derivative of the human IGF-1 and comprises an N-terminal 13-residue extension (also referred as IGF-1 LONG, SEQ ID NO: 3), a mutation E3R (SEQ ID NO: 4) or a mutation E3K (SEQ ID NO: 62) or a combination thereof (LONG E3R, also referred as LR3, SEQ ID NO: 6). In some embodiments, the IGF-1 variant comprises the E3R substitution, an N-terminal 13-residue extension, deletion of amino acids 1-3 ((Des1-3), SEQ ID NO: 5) or a combination thereof to decrease the binding of the activator domain to the IGF binding proteins which are present in the serum and other body fluid.

In some embodiments, the activator domain is a derivative of the human IGF-1 and comprises one or more of the following modifications: an N-terminal 13-residue extension (SEQ ID NO: 3), a deletion of amino acids 1-3 (SEQ ID NO: 5), a substitution replacing Arg for a Glu at the 3 position of the polypeptide (SEQ ID NO: 4), no Arginine at position 37 (R37X, SEQ ID NO: 7), a deletion of amino acids 68-70 (3X, SEQ ID NO: 8), or an N-terminal 13-residue extension and a substitution replacing Arg for a Glu at the 3 position of the wild-type polypeptide (SEQ ID NO: 6).

It is believed that the chimeric proteins that contain IGF-1 LONG, IGF-1 LONG E3R (referred to as IGF-1(LR3)) or IGF1 Des1-3, have decreased affinity for IGF binding proteins relative to wild-type IGF-1. In some embodiments, the IGF-1 variants of the chimeric proteins described herein can activate the signaling pathway while having a substantially decreased interaction with the IGF-1 binding proteins relative to wild-type IGF-1.

In some embodiments, the IGF-1 variant can be modified by glycosylation of one or more glyscosylation site present in the IGF-1 variant.

In some embodiments, the chimeric proteins that contain the IGF-1 variants described herein have a potency for non-target cells that is less than wild-type IGF-1 for non-target cells.

Certain IGF-1 activator domains that bind to growth factor receptors are provided herein in any of SEQ ID NOs: 1-8, 25-30, 62-67. Additional peptide sequence modifications can be included, such as variations, deletions, substitutions or derivatizations of the amino acid sequence of the sequences disclosed herein, so long as the peptide has substantially the same activity or function as the unmodified peptides. Notably, a modified peptide will retain activity or function associated with the unmodified peptide, the modified peptide will generally have an amino acid sequence “substantially homologous” with the amino acid sequence of the unmodified sequence.

In some embodiments, the IGF-1 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in any one of SEQ ID NOs: 1-8, 25-30, 62-67. In some embodiments, the IGF-1 variant can have an amino acid sequence having from about 85% to about 90%, from about 90% to about 95%, from about 95% to about 98%, from about 98% identity to about 99% identity to the amino acid sequence provided in any one of SEQ ID NOs: 1-8, 25-30, 62-67. In some embodiments, the IGF-1 variant can comprise 10, 20, 30, 40, 50, 60 or more consecutive amino acids of any one of amino acids in any one of SEQ ID NOs: 1-8, 25-30, 62-67. In some embodiments, the IGF-1 variant can have an amino acid sequence recited in any one of SEQ ID NOs: 1-8, 25-30, 62-67. In some embodiments, the IGF-1 variant can have an amino acid sequence recited in any one of SEQ ID NOs: 2-8, 25-30, 62-67. In some embodiments, the IGF-1 variant can have an amino acid sequence recited in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 25, SEQ ID NO: 26, SEQ ID NO: 27, SEQ ID NO: 28, SEQ ID NO: 29 or SEQ ID NO: 30, SEQ ID NO: 62, SEQ ID NO: 63, SEQ ID NO: 64, SEQ ID NO: 65, SEQ ID NO: 66, SEQ ID NO: 67.

Insulin-like growth factor 2 (IGF-2) plays an essential role in the regulation of cell proliferation, growth, migration, differentiation and survival. IGF2 binds to the non-signaling IGF type 2 receptor (IGF2R) with high affinity. IGF2 can also bind to different signaling receptors, such as the IGF type 1 receptor (IGF1R) and the insulin receptor.

In some embodiments, the activator domain comprises or consist of wild type human IGF-2 or fragment thereof. In some embodiments, the activator domain comprises or consists of an amino acid sequence according to SEQ ID NO: 9. In some embodiments, the activator domain comprises or consist of a variant of human IGF-2 or fragment thereof. In some embodiments, the IGF-2 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 9. In some embodiments, the IGF-2 variant can have an amino acid sequence having from about 85% to about 90%, from about 90% to about 95%, from about 95% to about 98%, from about 98% identity to about 99% identity to the amino acid sequence provided in SEQ ID NO: 9.

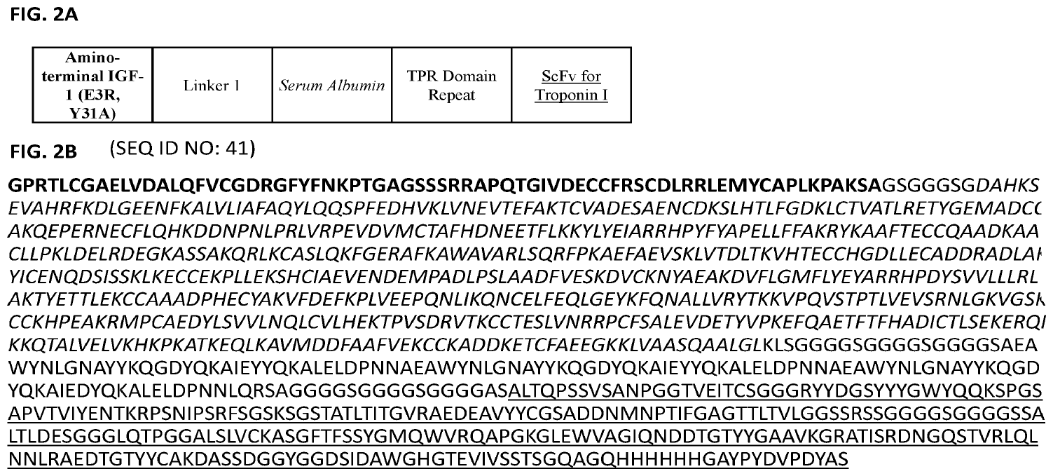

In some embodiments, the chimeric protein comprises an activator domain comprising or consisting of a IGF-1 variant (See FIG. 2A).

Suitable conservative substitutions of amino acids are known to those of skill in the art and may be made generally without altering the biological activity of the resulting molecule. Those of skill in this art recognize that, in general, single amino acid substitutions in non-essential regions of a polypeptide do not substantially alter biological activity.

In some embodiments, the activator domain comprises or consists of an amino acid sequence according to any of SEQ ID NOs: 1-9, 25-30, 62-67. In some embodiments, the activator domain comprises or consists of an amino acid sequence with an identity of at least 85%, at least 90%, at least 91%, at least 92%, at least 93%, at least 94%, at least 95%, at least 96%, at least 97%, at least 98%, at least 99% according to any of SEQ ID NOs: 1-9, 25-30, 62-67.

In some embodiments, the chimeric proteins that contain the IGF-1 or IGF-2 variants have a half maximal effective concentration (EC50) that is lower in damaged tissue than in healthy tissue. In some embodiments, the chimeric proteins that contain the IGF-1 or IGF-2 variants have a half maximal effective concentration (EC50) that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower in damaged tissue than in healthy tissue.

In some embodiments, the chimeric proteins provided herein having such variant growth factors have a higher specificity to the damaged tissue targeted or apoptotic cells.

### Fibroblast Growth Factor (FGF) and Derivatives Thereof

Fibroblast growth factor (FGF) is a growth factor which has shown the potential effects on the repair and regeneration of tissues. FGF-2 (also known as basic fibroblast growth factor) binds to FGF receptor (FGFR) and induces downstream signaling pathways, such as ERK pathway, that regulate cellular behavior. FGF-9 is known to play an important role in the regulation of embryonic development, cell proliferation, cell differentiation and cell migration. In some embodiment, the activator domain comprises FGF-2, FGF-9, a fragment thereof, or a variant thereof.

In some embodiments, the activator domain comprises or consist of wild type human FGF-2 or fragment thereof. In some embodiments, the activator domain comprises or consists of an amino acid sequence according to SEQ ID NO: 10. In some embodiments, the activator domain comprises or consist of a variant of human FGF-2 or fragment thereof. In some embodiments, the FGF-2 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 10. In some embodiments, the FGF-2 variant can have an amino acid sequence having from about 85% to about 90%, from about 90% to about 95%, from about 95% to about 98%, from about 98% identity to about 99% identity to the amino acid sequence provided in SEQ ID NO: 10.

In some embodiments, the activator domain comprises or consist of wild type human FGF-9 or fragment thereof. In some embodiments, the activator domain comprises or consists of an amino acid sequence according to SEQ ID NO: 11. In some embodiments, the activator domain comprises or consist of a variant of human FGF-9 or fragment thereof. In some embodiments, the FGF-2 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 11. In some embodiments, the FGF-9 variant can have an amino acid sequence having from about 85% to about 90%, from about 90% to about 95%, from about 95% to about 98%, from about 98% identity to about 99% identity to the amino acid sequence provided in SEQ ID NO: 11.

### Bone Morphogenetic Protein (BMP) and Derivatives Thereof

Bone morphogenetic protein BMP2 and BMP7 belong to the TGF-β superfamily. BMP2 is a regulator of cell growth and differentiation. BMP-7 has been found to protect renal cells from fibrosis in acute and chronic renal injury.

In some embodiment, the activator domain comprises BMP2, BMP7, a fragment thereof, or a variant thereof.

In some embodiments, the activator domain comprises or consist of wild type human BMP2 or fragment thereof. In some embodiments, the activator domain comprises or consists of an amino acid sequence according to SEQ ID NO: 38. In some embodiments, the activator domain comprises or consist of a variant of human BMP2 or fragment thereof. In some embodiments, the BMP2 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 38. In some embodiments, the BMP2 variant can have an amino acid sequence having from about 85% to about 90%, from about 90% to about 95%, from about 95% to about 98%, from about 98% identity to about 99% identity to the amino acid sequence provided in SEQ ID NO: 38.

In some embodiments, the activator domain comprises or consist of wild type human BMP7 or fragment thereof. In some embodiments, the activator domain comprises or consists of an amino acid sequence according to SEQ ID NO: 39. In some embodiments, the activator domain comprises or consist of a variant of human BMP7 or fragment thereof. In some embodiments, the BMP7 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 39. In some embodiments, the BMP7 variant can have an amino acid sequence having from about 85% to about 90%, from about 90% to about 95%, from about 95% to about 98%, from about 98% identity to about 99% identity to the amino acid sequence provided in SEQ ID NO: 39.

### Vascular Endothelial Growth Factor a (VEGF-A) and Derivatives Thereof

Vascular endothelial growth factor A (VEGF-A) has been shown to be essential or maintaining the glomerular filtration barrier by regulating the survival and structure of the endothelial cells and podocytes.

In some embodiment, the activator domain comprises VEGF-A, a fragment thereof, or a variant thereof.

In some embodiments, the activator domain comprises or consist of wild type human VEGF-A or fragment thereof. In some embodiments, the activator domain comprises or consists of an amino acid sequence according to SEQ ID NO: 40. In some embodiments, the activator domain comprises or consist of a variant of human VEGF-A or fragment thereof. In some embodiments, the VEGF-A variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 40. In some embodiments, the VEGF-A variant can have an amino acid sequence having from about 85% to about 90%, from about 90% to about 95%, from about 95% to about 98%, from about 98% identity to about 99% identity to the amino acid sequence provided in SEQ ID NO: 40.

### Neuregulin (NRG) and Derivatives Thereof

As used herein the term “neuregulin” or NRG refers to a member of a family of structurally related proteins that are part of the EGF family of proteins. Multiple family members are generated by alternate splicing or by use of several cell type-specific transcription initiation sites. In general, they bind to and activate the erbB family of receptor tyrosine kinases (ErbB-2 (HER2), ErbB-3 (HER3), and ErbB-4 (HER4)), functioning both as heterodimers and homodimers.

In some embodiments, the activator domain comprises or consists of neuregulin, neuregulin Beta1, neuregulin alpha, neuregulin alphalA, neuregulin 3, neuregulin beta2, fragment thereof or variants thereof. In some embodiments, the EGF-like domain of neuregulin-1 takes part in signal transduction by stimulating the ErbB receptor tyrosine kinase ErbB2.

In some embodiments, the neuregulin protein can bind and activate ErbB-2 (as part of a heterodimer with ErbB-3, ErbB-4) and include but not limited to all neuregulin isoforms, neuregulin EGF domain alone, polypeptides comprising neuregulin EGF-like domain, neuregulin mutants or derivatives, and any kind of neuregulin-like gene expression products that can activate erbb-2.

In some embodiments, the activator domain comprises or consist of wild type human neuregulin, neuregulin Beta1, neuregulin alpha, neuregulin alphalA, neuregulin 3, neuregulin beta2 or fragment thereof. In some embodiments, the activator domain comprises or consists of an amino acid sequence according to any one of SEQ ID NOs: 31-37. In some embodiments, the activator domain comprises or consist of a variant of human neuregulin, neuregulin beta1, neuregulin alpha, neuregulin alphalA, neuregulin 3, neuregulin beta2 or fragment thereof.

In some embodiments, the NRG variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in any one of SEQ ID NOs: 31-37. In some embodiments, the NRG variant can have an amino acid sequence having from about 85% to about 90%, from about 90% to about 95%, from about 95% to about 98%, from about 98% identity to about 99% identity to the amino acid sequence provided in any one of SEQ ID NOs: 31-37. In some embodiments, the NRG variant can comprise 10, 20, 30, 40, 50, 60 or more consecutive amino acids of any one of amino acids in any one of SEQ ID NOs: 31-37. In some embodiments, the NRG variant can have an amino acid sequence recited in any one of SEQ ID NOs: 31-37. In some embodiments, the NRG variant can have an amino acid sequence recited in any one of SEQ ID NOs: 31-37. In some embodiments, the NRG variant can have an amino acid sequence recited in, SEQ ID NO: 31, SEQ ID NO: 32, SEQ ID NO: 33, SEQ ID NO: 34, SEQ ID NO: 35, SEQ ID NO: 36, or SEQ ID NO: 37.

In some embodiments, the variant of human neuregulin Beta1 comprises a GI S substitution. In some embodiments, the variant of human neuregulin Beta1 comprises or consists of SEQ ID NO: 32.

Suitable conservative substitutions of amino acids are known to those of skill in the art and may be made generally without altering the biological activity of the resulting molecule. Those of skill in this art recognize that, in general, single amino acid substitutions in non-essential regions of a polypeptide do not substantially alter biological activity.

In some embodiments, the activator domain comprises or consists of an amino acid sequence according to any of SEQ ID NOs: 31-37. In some embodiments, the activator domain comprises or consists of an amino acid sequence with an identity of at least 85%, at least 90%, at least 91%, at least 92%, at least 93%, at least 94%, at least 95%, at least 96%, at least 97%, at least 98%, at least 99% according to any of SEQ ID NOs: 31-37.

### Half-Life Modulator

One skilled in the art would appreciate that proteins used in therapeutic applications may not exhibit optimal serum half-lives due to their relatively low molecular weight. In some therapeutic applications, it may therefore be desirable to extend the half-life of the proteins. In some embodiments, to achieve accumulation of the chimeric protein to the diseased injured or damaged area of an organ, the chimeric protein is conjugated operatively associated or fused with a half-life modulator. Preferably, the half-life modulator is non-immunogenic peptide.

For example, short half-life is the most limiting attribute of wild-type growth factors as therapeutics. Intravenous administered IGF-1 has a serum half-life in humans of less than 1 hour. The extended half-life of chimeric proteins disclosed herein compared to IGF-1, for example, allows for 1) equivalent efficacy with less frequent dosing; 2) equivalent exposure at a lower dose; 3) lower Cmax at an equivalent exposure level, reducing the risk of Cmax-related toxicity.

In some embodiments, the half-life modulators can increase the in vivo half-life of the chimeric proteins. For example, the half-life of the chimeric proteins comprising the half-life modulator is about 1 hour, 2 hour, 3 hours, 4 hours, 5 hours, 6 hours or greater. For example, the half-life of the chimeric proteins can be about 8 hours or more when tested in cynomolgus monkey. In some embodiments, the half-life of the chimeric proteins comprising the half-life modulator is about 24 hours, or greater. In some embodiments, the half-life of the chimeric proteins comprising the half-life modulator is about a week or greater.

In some embodiments, the half-life modulator is non-immunogenic in humans.

In some embodiments, the half-life modulator is a peptide that interacts with cellular machinery that promote evasion of lysosomal degradation pathways (e.g.—FcRn receptor-mediated recycling).

### Human Serum Albumin

In some embodiments, the half-life modulator is designed to extend the half-life of the chimeric protein through binding to serum components such as Human Serum Albumin (HSA). HSA is the most abundant protein in the blood and has a demonstrated safety in humans.

In some embodiments, the half-life modulator is a HSA variant. In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild type human serum albumin amino acid sequence (wtHSA, SEQ ID NO: 12). In some embodiments, the half-life modulator comprises at least 200 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 300 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 400 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 500 consecutive amino acids that are at least 70%, 80%, 85%, 90%, 91%, 92%, 93%, 94%, 95%, 96%, 97%, 98%, or 99% identical to wild type human serum albumin amino acid sequence.

In some embodiments, the HSA variant can have one of more of the following substitutions:
cysteine C58 can be substituted, for example, with a serine (C58S),
lysine K420 can be substituted for example, with a glutamic acid (K420E),
asparagine N527 can be substituted for example, with a glutamine (N527Q),
glutamic acid E505 can be substituted for example, with a glycine G (E505G),
valine V547 can be substituted for example, with an alanine (V547A),
asparagine N527 can be substituted for example, with a Glutamine (N527Q).

In some embodiments, the HSA variant can have amino acids 26-609 and have one of more of the following substitutions:
cysteine C58 can be substituted for example, with a serine (C58S),
lysine K420 can be substituted for example, with a glutamic acid (K420E),
asparagine N527 can be substituted for example, with a glutamine (N527Q),
glutamic acid E505 can be substituted for example, with a glycine G (E505G),
valine V547 can be substituted for example, with an alanine (V547A),
asparagine N503 and/or N527 can be substituted for example, with an Glutamine (N503Q and/or N527Q).

In some embodiments, the HSA variant (referred herein as mHSA) has the following substitutions: C34S, N503Q (SEQ ID NO: 13). In some embodiments, the HSA variant (referred herein as mHSA7) has the following substitutions C34S, N503Q, E505G and V547A (SEQ ID NO: 14). In some embodiments, the HSA variant has amino acids 26-609 and the following substitutions C58S and N527Q (SEQ ID NO: 15). In some embodiments, the HSA variant has the following substitutions: C34S, N503Q and has an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 13. In some embodiments, the HSA variant has the following substitutions C34S, N503Q, E505G and V547A and has an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 14 or SEQ ID NO: 15.

In some embodiments, the asparagine at position 503 and/or 527 of HSA, which may be deamidated and decrease half-life, can be removed by the N503Q substitution and/or the N527Q. In some embodiments, the cysteine C34 of HSA may be substituted to serine or alanine (S or A) to remove the free cysteine and minimize alternate disulfide-bond formation.

In some embodiments, the half-life modulator is a modified version of the domain III (mHSA_dIII) of a modified HSA with the N503Q substitution and an additional terminal glycine. Such a modified version retains the HSA property of binding to FcRn and increased serum half-life.

### Other Half-Life Modulators

In some embodiments, the half-life modulator is Fc domain of an antibody or a single chain constant fragment. In some embodiments, the half-life modulator comprises Fc regions of an immunoglobulin molecule (e.g. IgG). In some embodiments, the half-life modulator comprises at least 100 consecutive amino acids that are at least 70%, 80%, 85%, 90%, 91%, 92%, 93%, 94%, 95%, 96%, 97%, 98%, or 99% identical to a human Fc amino acid sequence. The Fc domain of an antibody has a natural capability to bind FcRn, resulting in an extended half-life. In some embodiments, the Fc domain of an antibody is engineered not to bind Fc(gamma)R In an exemplary embodiment, the Fc domain is engineered to substitute N297 with Q (N297Q variant). In some embodiments, the half-life modulator is a monomeric variant form of Fc (scFc). For example, the subset of IgG heavy chain which naturally dimerizes to form Fc is hinge-CH2-CH3. In some embodiments, the Fc domain is engineered to form a single chain by linking the hinge-CH2-CH3 with a flexible linker such as GGGGSGGGGSGGGGSGGGGS (SEQ ID NO: 16) to create a hinge-CH2-CH3-linker-hinge-CH2-CH3 chain. In an exemplary embodiment, the single chain Fc (scFc) is engineered to substitute N297 with Q and C220 with S (N297Q, C220S).

In some embodiments, the half-life modulator is a single chain variable fragment (scFv) of an antibody targeted to albumin or other circulating protein. In some embodiments, the half-life modulator comprises an amino acid sequence that is at least 70%, 80%, 85%, 90%, 91%, 92%, 93%, 94%, 95%, 96%, 97%, 98%, or 99% identical to scFv amino acid sequence directed to a specific antigen, such as, but not limited to, albumin. In some embodiments, the half-life modulator comprises at least 50, at least 100, at least 150, at least 200, at least 250 consecutive amino acids that are at least 70%, 80%, 85%, 90%, 91%, 92%, 93%, 94%, 95%, 96%, 97%, 98%, or 99% identical to a scFv amino acid sequence directed to a specific antigen, such as, but not limited to, albumin.

In some embodiments, the half-life modulator is transferrin such as human transferrin (Tf, SEQ ID NO: 17). In some embodiments, the half-life modulator comprises an amino acid sequence that is at least 70%, 80%, 85%, 90%, 91%, 92%, 93%, 94%, 95%, 96%, 97%, 98%, or 99% identical to human transferrin amino acid sequence. In some embodiments, the half-life modulator comprises at least 100, at least 200, at least 300, at least 400, at least 500, at least 600, at least 650 consecutive amino acids that are at least 70%, 80%, 85%, 90%, 91%, 92%, 93%, 94%, 95%, 96%, 97%, 98%, or 99% identical to a human transferrin amino acid sequence.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild type human alpha-fetoprotein amino acid sequence (AFP, SEQ ID NO: 18). In some embodiments, the half-life modulator comprises at least 100 consecutive amino acids that are about 70%, 80%, 85%, 90%, 91%, 92%, 93%, 94%, 95%, 96%, 97%, 98%, or 99% identical to wild type human alpha-fetoprotein (AFP) amino acid sequence. In some embodiments, the N-linked glycosylation site of the AFP is removed by the N251Q substitution.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical wild-type vitamin D-binding protein amino acid sequence (VDBP, SEQ ID NO: 19). In some embodiments, the half-life modulator comprises at least 100 consecutive amino acids that are about 70%, 80%, 85%, 90%, 91%, 92%, 93%, 94%, 95%, 96%, 97%, 98%, or 99% identical wild-type vitamin D-binding protein (VDBP) amino acid sequence. In some embodiments, the N-linked glycosylation site of the VDBP can be removed by the N288Q or N288T substitution.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild-type human transthyretin amino acid sequence (TTR, SEQ ID NO: 20). In some embodiments, the half-life modulator comprises at least 100 consecutive amino acids that are about 70%, 80%, 85%, 90%, 91%, 92%, 93%, 94%, 95%, 96%, 97%, 98%, or 99% identical to wild type human transthyretin (TTR) amino acid sequence. In some embodiments, the transthyretin is modified to remove the N118 N-glycosylation site. In some embodiments, the half-life modulator is a monomeric form of TTR.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to a PASylation amino acid sequence. PASylation are proline-, alanine-, and/or serine-rich sequences that mimic PEGylation (see WO/2008/155134).

In some embodiments, the half-life modulator comprises at least 100 consecutive amino acids that are about 70%, 80%, 85%, 90%, 91%, 92%, 93%, 94%, 95%, 96%, 97%, 98%, or 99% identical to a PASylation amino acid sequence. PASylation are proline-, alanine-, and/or serine-rich sequences that mimic PEGylation. Polypeptide stretches of proline, alanine, and/or serine form semi-structured three-dimensional domains with large hydrodynamic radius, thereby reducing clearance of fusion proteins. In some embodiments, the PASylation amino acid sequence is about 200, 300, 400, 500 or 600 amino acids long. For example, the PASylation is a 20 times repeat of the amino acid sequence ASPAAPAPASPAAPAPSAPA (SEQ ID NO: 21).

In some embodiments, the half-life modulator comprises the attachment of polyethylene glycol (PEG) chain or chains to the fusion proteins through chemical attachment either to the N- and/or C-terminus and/or to an amino acid side chain (e.g., PEG-maleimide attachment to cysteines). PEG chains form semi-structured three-dimensional domains with large hydrodynamic radius, thereby reducing clearance of fusion proteins.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to an albumin-binding domain human antibody (albudAb) amino acid sequence (SEQ ID NO: 22). In some embodiments, the half-life modulator comprises at least 100 consecutive amino acids that are about 70%, 80%, 85%, 90%, 91%, 92%, 93%, 94%, 95%, 96%, 97%, 98%, or 99% identical to an albumin-binding domain human antibody (albudAb) amino acid sequence. Albumin-binding domain antibodies can increase the fusion protein half-life by binding non-covalently to serum albumin (see WO2008/096158). In some embodiments, the albumin-binding domain human antibody is engineered to remove the C-terminal arginine to remove the Lys-Arg Kex2 protease site. Representative such half-life modulators include those recited in any one of SEQ ID NOs: 12-15, 17-22. In some embodiments, the half-life modulators can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NOs: 12-15, 17-22.

In some embodiments, the half-life modulators can be modified to substitute the cysteine residues to serine or alanine residues to reduce the ability to form disulfide bonds.

In some embodiments, the targeting domain and activator domain can be joined via a half-life modulator. Accordingly, the half-life modulator can have two termini, an N-terminus and a C-terminus. In some embodiments, the half-life modulator is joined at one terminus via a peptide bond to the targeting polypeptide domain and is joined at the other terminus via a peptide bond to the activator domain. In certain embodiments, the half-life modulator is joined at the N-terminus to the C-terminus of the targeting polypeptide domain and at the C-terminus to the N-terminus of the activator domain. In other embodiments, the half-life modulator is joined at the C-terminus to the targeting polypeptide domain and at the N-terminus to the activator domain. Yet, in other embodiments, the half-life modulator is joined at one of the termini of the chimeric protein. For example, in some embodiments, the half-life modulator is joined at the C-terminus to the N-terminus of the activator domain. In other embodiments, the half-life modulator is joined at the N-terminus to the C-terminus of the targeting domain. In other embodiments, the half-life modulator can be joined at the N-terminus to the C-terminus of the activator domain. Yet in other embodiments, the half-life modulator can be joined at the N-terminus to the C-terminus of the targeting domain.

### Peptide Linkers

In some embodiments, the activator domain and the half-life modulator, and targeting domain and the half-life modulator are linked by a peptide linker (e.g., from 2 to 40, 2-50, 2-100, 2-200, 2-400, 2-500 amino acid residues) such that upon target recognition and engagement by the targeting domain, the presentation of the activator domain is optimized for binding to and activation of extracellular receptors on the surface of cells that present the target at a given surface density (e.g.—5×10² molecules/1,000 A2).

Targeted delivery of the activator domain, including but not limited to IGF-1 or variant thereof, for the activation of receptors on cells or tissues displaying a specific target requires appropriate presentation of both the activator domain and the targeting domain. In some embodiments, the flexibility of the linker is optimized for proper geometry of the engaged chimeric protein. Some of the principal determinants of the geometric constraints are the distances from the cell surface for the target and the receptor.

Additional optimization can be driven by the relative number of receptors and target molecules. At high ratios of Receptor:Target molecule, the engagement of both domains is reaction-limited. When the target molecule is more abundant than the receptor, the occupancy of both domains is diffusion-limited. Under the reaction-limit, optimal delivery of the activator domain is attained via short and rigid linkers. Under the diffusion limit, long and flexible linkers allow the activator domain to access a larger surface area. For cells with complex shapes (i.e. bodies and neuronal processes) and receptor distributions, appropriate design of linker flexibility can enable precise targeting to sub-cellular regions.

In some embodiments, the peptide linker is present at the N-terminus, at the C-terminus or at both the N-terminus and the C-terminus of the half-life modulator. In some embodiments, the chimeric protein comprises a half-life modulator comprising a first peptide linker at the N-terminus of the half-life modulator and a second peptide linker at the C-terminus of the half-life modulator. In some embodiments, the chimeric protein comprises a polypeptide domain comprising a first peptide linker at the N-terminus of a human serum albumin or variant thereof and a second peptide linker at the C-terminus of the human serum albumin or variant thereof. In some embodiments, the variant of human serum albumin consists of or comprises SEQ ID NO: 15. In some embodiments, the variant of human serum albumin consists of or comprises of an amino acid sequences having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 15. In some embodiments, the variant of human serum albumin consists of or comprises of an amino acid sequences having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 13. In some embodiments, the variant of human serum albumin consists of or comprises of an amino acid sequences having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 14. In some embodiments, the variant of human serum albumin consists of or comprises of an amino acid sequences having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 12.

In some embodiments, the linker sequence can be a flexible linker sequence. Flexible linkers allow adjacent protein domains to be free to move relative to one another. Longer linkers may be used when it is desirable to ensure that two adjacent domains do not sterically interfere with one another. In some embodiments, such peptide linkers are flexible (for example glycine-rich). In some embodiments, the linker sequence can be a non-flexible linker sequence. For example, the linker sequence can be structured (e.g., alpha-helix rich). In some examples, the linker sequence is a synthetic linker sequence. In some embodiments, any of the chimeric proteins described herein can include one, two, or more linker sequence(s). In some embodiments, the two or more linker sequences can be the same or can be different linker sequences, e.g., any of the exemplary linker sequences described herein or known in the art. In some embodiments, the two or more linker sequences can comprise two or more flexible linkers or can comprise a flexible linker and a non-flexible linker. Linkers may be selected to, for example, ensure that the targeting domain and the activator domain do not sterically interfere with one another.

In some embodiments, the peptide linkers are longer than 2 amino acids. For example, the peptide linkers are 5, 6, 7, 8, 9, 10, 11, 12, 13, 14, 15, 16, 17, 18, 19 or 20 amino acids long or longer. In some embodiments, the peptide linkers are 20 or more, 30 or more, 40 or more, 50 or more, 60 or more, 70 or more, 80 or more, 90 or more, 100 or more, 110 or more, 120 or more, 130 or more, 140 or more, 150 or more, 160 or more, 170 or more, 180 or more, 190 or more, 200 or more, 210 amino or more, 220 or more, 230 or more, 240 or more, 250 or more, 260 or more, 270 or more, 280 or more, 290 or more, 300 or more, 310 or more, 320 or more, 330 or more, 340 or more, 350 or more, 360 or more, 370 or more, 380 or more, 390 or more, 400 or more, 410 or more, 420 or more, 430 or more, 440 or more, 450 or more, 460 or more, 470 or more, 480 or more, 490 or more, 500 or more amino acids long. In some embodiments, the peptide linkers are from 5 up to 10, up to 20, up to 30, up to 40, up to 50, up to 60, up to 70, up to 80, up to 90, up to 100, up to 110, up to 120, up to 130, up to 140, up to 150, up to 160, up to 170, up to 180, up to 190, up to 200, up to 210 amino, up to 220, up to 230, up to 240, up to 250, up to 260, up to 270, up to 280, up to 290, up to 300, up to 310, up to 320, up to 330, up to 340, up to 350, up to 360, up to 370, up to 380, up to 390, up to 400, up to 410, up to 420, up to 430, up to 440, up to 450, up to 460, up to 470, up to 480, up to 490, up to 500 amino acids long.

In some embodiments, a linker sequence can have a total length of 1 amino acid to about 1 amino acid to about 500 amino acids, 1 amino acid to about 450 amino acids, 1 amino acid to about 425 amino acids, 1 amino acid to about 400 amino acids, 1 amino acid to about 375 amino acids, 1 amino acid to about 350 amino acids, 1 amino acid to about 325 amino acids, 1 amino acid to about 300 amino acids, 1 amino acid to about 275 amino acids, 1 amino acid to about 250 amino acids, 1 amino acid to about 240 amino acids, 1 amino acid to about 220 amino acids, 1 amino acid to about 200 amino acids, 1 amino acid to about 180 amino acids, 1 amino acid to about 160 amino acids, 1 amino acid to about 140 amino acids, 1 amino acid to about 120 amino acids, 1 amino acid to about 100 amino acids, 1 amino acid to about 80 amino acids, 1 amino acid to about 60 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 24 amino acids, 1 amino acid to about 22 amino acids, 1 amino acid to about 20 amino acids, 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, about 2 amino acids to about 500 amino acids, about 2 amino acids to about 475 amino acids, about 2 amino acids to about 450 amino acids, about 2 amino acids to about 425 amino acids, about 2 amino acids to about 400 amino acids, about 2 amino acids to about 375 amino acids, about 2 amino acids to about 350 amino acids, about 2 amino acids to about 325 amino acids, about 2 amino acids to about 300 amino acids, about 2 amino acids to about 275 amino acids, about 2 amino acids to about 250 amino acids, about 2 amino acids to about 240 amino acids, about 2 amino acids to about 220 amino acids, about 2 amino acids to about 200 amino acids, about 2 amino acids to about 180 amino acids, about 2 amino acids to about 160 amino acids, about 2 amino acids to about 140 amino acids, about 2 amino acids to about 120 amino acids, about 2 amino acids to about 100 amino acids, about 2 amino acids to about 80 amino acids, about 2 amino acids to about 60 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 4 amino acids, about 3 amino acids to about 500 amino acids, about 3 amino acids to about 475 amino acids, about 2 amino acids to about 450 amino acids, about 3 amino acids to about 425 amino acids, about 3 amino acids to about 400 amino acids, about 3 amino acids to about 375 amino acids, about 3 amino acids to about 350 amino acids, about 3 amino acids to about 325 amino acids, about 3 amino acids to about 300 amino acids, about 3 amino acids to about 275 amino acids, about 3 amino acids to about 250 amino acids, about 3 amino acids to about 240 amino acids, about 3 amino acids to about 220 amino acids, about 3 amino acids to about 200 amino acids, about 3 amino acids to about 180 amino acids, about 3 amino acids to about 160 amino acids, about 3 amino acids to about 140 amino acids, about 3 amino acids to about 120 amino acids, about 3 amino acids to about 100 amino acids, about 3 amino acids to about 80 amino acids, about 3 amino acids to about 60 amino acids, about 3 amino acids to about 50 amino acids, about 2 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 24 amino acids, about 3 amino acids to about 22 amino acids, about 3 amino acids to about 20 amino acids, about 3 amino acids to about 18 amino acids, about 3 amino acids to about 16 amino acids, about 3 amino acids to about 14 amino acids, about 3 amino acids to about 12 amino acids, about 3 amino acids to about 10 amino acids, about 3 amino acids to about 8 amino acids, about 3 amino acids to about 6 amino acids, about 3 amino acids to about 4 amino acids, about 4 amino acids to about 500 amino acids, about 4 amino acids to about 475 amino acids, about 4 amino acids to about 450 amino acids, about 4 amino acids to about 425 amino acids, about 4 amino acids to about 400 amino acids, about 4 amino acids to about 375 amino acids, about 4 amino acids to about 350 amino acids, about 4 amino acids to about 325 amino acids, about 4 amino acids to about 300 amino acids, about 4 amino acids to about 275 amino acids, about 4 amino acids to about 250 amino acids, about 4 amino acids to about 240 amino acids, about 4 amino acids to about 220 amino acids, about 4 amino acids to about 200 amino acids, about 4 amino acids to about 180 amino acids, about 4 amino acids to about 160 amino acids, about 4 amino acids to about 140 amino acids, about 4 amino acids to about 120 amino acids, about 4 amino acids to about 100 amino acids, about 4 amino acids to about 80 amino acids, about 4 amino acids to about 60 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 5 amino acids to about 500 amino acids, about 5 amino acids to about 475 amino acids, about 5 amino acids to about 450 amino acids, about 5 amino acids to about 425 amino acids, about 5 amino acids to about 400 amino acids, about 5 amino acids to about 375 amino acids, about 5 amino acids to about 350 amino acids, about 5 amino acids to about 300 amino acids, about 5 amino acids to about 300 amino acids, about 5 amino acids to about 275 amino acids, about 5 amino acids to about 250 amino acids, about 5 amino acids to about 240 amino acids, about 5 amino acids to about 220 amino acids, about 5 amino acids to about 200 amino acids, about 5 amino acids to about 180 amino acids, about 5 amino acids to about 160 amino acids, about 5 amino acids to about 140 amino acids, about 5 amino acids to about 120 amino acids, about 5 amino acids to about 100 amino acids, about 5 amino acids to about 80 amino acids, about 5 amino acids to about 60 amino acids, about 5 amino acids to about 40 amino acids, about 5 amino acids to about 35 amino acids, about 5 amino acids to about 30 amino acids, about 5 amino acids to about 25 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 14 amino acids, about 5 amino acids to about 12 amino acids, about 5 amino acids to about 10 amino acids, about 5 amino acids to about 8 amino acids, about 5 amino acids to about 6 amino acids, about 6 amino acids to about 500 amino acids, about 6 amino acids to about 475 amino acids, about 6 amino acids to about 450 amino acids, about 6 amino acids to about 425 amino acids, about 6 amino acids to about 400 amino acids, about 6 amino acids to about 375 amino acids, about 6 amino acids to about 350 amino acids, about 6 amino acids to about 300 amino acids, about 6 amino acids to about 300 amino acids, about 6 amino acids to about 275 amino acids, about 6 amino acids to about 250 amino acids, about 6 amino acids to about 225 amino acids, about 6 amino acids to about 200 amino acids, about 6 amino acids to about 175 amino acids, about 6 amino acids to about 150 amino acids, about 6 amino acids to about 125 amino acids, about 6 amino acids to about 100 amino acids, about 6 amino acids to about 75 amino acids, about 6 amino acids to about 60 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 14 amino acids, about 6 amino acids to about 12 amino acids, about 6 amino acids to about 10 amino acids, about 6 amino acids to about 8 amino acids, about 7 amino acids to about 500 amino acids, about 7 amino acids to about 475 amino acids, about 7 amino acids to about 450 amino acids, about 7 amino acids to about 425 amino acids, about 7 amino acids to about 400 amino acids, about 7 amino acids to about 375 amino acids, about 7 amino acids to about 350 amino acids, about 7 amino acids to about 300 amino acids, about 7 amino acids to about 300 amino acids, about 7 amino acids to about 275 amino acids, about 7 amino acids to about 250 amino acids, about 7 amino acids to about 225 amino acids, about 7 amino acids to about 200 amino acids, about 7 amino acids to about 175 amino acids, about 7 amino acids to about 150 amino acids, about 7 amino acids to about 125 amino acids, about 7 amino acids to about 100 amino acids, about 7 amino acids to about 75 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 24 amino acids, about 7 amino acids to about 22 amino acids, about 7 amino acids to about 20 amino acids, about 7 amino acids to about 18 amino acids, about 7 amino acids to about 16 amino acids, about 7 amino acids to about 14 amino acids, about 7 amino acids to about 12 amino acids, about 7 amino acids to about 10 amino acids, about 7 amino acids to about 8 amino acids, about 8 amino acids to about 500 amino acids, about 8 amino acids to about 475 amino acids, about 8 amino acids to about 450 amino acids, about 8 amino acids to about 425 amino acids, about 8 amino acids to about 400 amino acids, about 8 amino acids to about 375 amino acids, about 8 amino acids to about 350 amino acids, about 8 amino acids to about 300 amino acids, about 8 amino acids to about 300 amino acids, about 8 amino acids to about 275 amino acids, about 8 amino acids to about 250 amino acids, about 8 amino acids to about 225 amino acids, about 8 amino acids to about 200 amino acids, about 8 amino acids to about 175 amino acids, about 8 amino acids to about 150 amino acids, about 8 amino acids to about 125 amino acids, about 8 amino acids to about 100 amino acids, about 8 amino acids to about 75 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 9 amino acids to about 500 amino acids, about 9 amino acids to about 475 amino acids, about 9 amino acids to about 450 amino acids, about 9 amino acids to about 425 amino acids, about 9 amino acids to about 400 amino acids, about 9 amino acids to about 375 amino acids, about 9 amino acids to about 350 amino acids, about 9 amino acids to about 300 amino acids, about 9 amino acids to about 300 amino acids, about 9 amino acids to about 275 amino acids, about 9 amino acids to about 250 amino acids, about 9 amino acids to about 225 amino acids, about 9 amino acids to about 200 amino acids, about 9 amino acids to about 175 amino acids, about 9 amino acids to about 150 amino acids, about 9 amino acids to about 125 amino acids, about 9 amino acids to about 100 amino acids, about 9 amino acids to about 75 amino acids, about 9 amino acids to about 50 amino acids, about 9 amino acids to about 45 amino acids, about 9 amino acids to about 40 amino acids, about 9 amino acids to about 35 amino acids, about 9 amino acids to about 30 amino acids, about 9 amino acids to about 25 amino acids, about 9 amino acids to about 24 amino acids, about 9 amino acids to about 22 amino acids, about 9 amino acids to about 20 amino acids, about 9 amino acids to about 18 amino acids, about 9 amino acids to about 16 amino acids, about 9 amino acids to about 14 amino acids, about 9 amino acids to about 12 amino acids, about 9 amino acids to about 10 amino acids, about 10 amino acids to about 500 amino acids, about 10 amino acids to about 475 amino acids, about 10 amino acids to about 450 amino acids, about 10 amino acids to about 425 amino acids, about 10 amino acids to about 400 amino acids, about 10 amino acids to about 375 amino acids, about 10 amino acids to about 350 amino acids, about 10 amino acids to about 325 amino acids, about 10 amino acids to about 300 amino acids, about 10 amino acids to about 275 amino acids, about 10 amino acids to about 250 amino acids, about 10 amino acids to about 225 amino acids, about 10 amino acids to about 200 amino acids, about 10 amino acids to about 175 amino acids, about 10 amino acids to about 150 amino acids, about 10 amino acids to about 125 amino acids, about 10 amino acids to about 100 amino acids, about 10 amino acids to about 75 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 11 amino acids to about 500 amino acids, about 11 amino acids to about 475 amino acids, about 11 amino acids to about 450 amino acids, about 11 amino acids to about 425 amino acids, about 11 amino acids to about 400 amino acids, about 11 amino acids to about 375 amino acids, about 11 amino acids to about 350 amino acids, about 11 amino acids to about 325 amino acids, about 11 amino acids to about 300 amino acids, about 11 amino acids to about 275 amino acids, about 11 amino acids to about 250 amino acids, about 11 amino acids to about 225 amino acids, about 11 amino acids to about 200 amino acids, about 11 amino acids to about 175 amino acids, about 11 amino acids to about 150 amino acids, about 11 amino acids to about 125 amino acids, about 11 amino acids to about 100 amino acids, about 11 amino acids to about 75 amino acids, about 11 amino acids to about 50 amino acids, about 11 amino acids to about 45 amino acids, about 11 amino acids to about 40 amino acids, about 11 amino acids to about 35 amino acids, about 11 amino acids to about 30 amino acids, about 11 amino acids to about 25 amino acids, about 11 amino acids to about 24 amino acids, about 11 amino acids to about 22 amino acids, about 11 amino acids to about 20 amino acids, about 11 amino acids to about 18 amino acids, about 11 amino acids to about 16 amino acids, about 11 amino acids to about 14 amino acids, about 11 amino acids to about 12 amino acids, about 12 amino acids to about 500 amino acids, about 12 amino acids to about 475 amino acids, about 12 amino acids to about 450 amino acids, about 12 amino acids to about 425 amino acids, about 12 amino acids to about 400 amino acids, about 12 amino acids to about 375 amino acids, about 12 amino acids to about 350 amino acids, about 12 amino acids to about 325 amino acids, about 12 amino acids to about 300 amino acids, about 12 amino acids to about 275 amino acids, about 12 amino acids to about 250 amino acids, about 12 amino acids to about 225 amino acids, about 12 amino acids to about 200 amino acids, about 12 amino acids to about 175 amino acids, about 12 amino acids to about 150 amino acids, about 12 amino acids to about 125 amino acids, about 12 amino acids to about 100 amino acids, about 12 amino acids to about 75 amino acids, about 12 amino acids to about 50 amino acids, about 12 amino acids to about 45 amino acids, about 12 amino acids to about 40 amino acids, about 12 amino acids to about 35 amino acids, about 12 amino acids to about 30 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 14 amino acids, about 13 amino acids to about 500 amino acids, about 13 amino acids to about 475 amino acids, about 13 amino acids to about 450 amino acids, about 13 amino acids to about 425 amino acids, about 13 amino acids to about 400 amino acids, about 13 amino acids to about 375 amino acids, about 13 amino acids to about 350 amino acids, about 13 amino acids to about 325 amino acids, about 13 amino acids to about 300 amino acids, about 13 amino acids to about 275 amino acids, about 13 amino acids to about 250 amino acids, about 13 amino acids to about 225 amino acids, about 13 amino acids to about 200 amino acids, about 13 amino acids to about 175 amino acids, about 13 amino acids to about 150 amino acids, about 13 amino acids to about 125 amino acids, about 13 amino acids to about 100 amino acids, about 13 amino acids to about 75 amino acids, about 13 amino acids to about 50 amino acids, about 13 amino acids to about 45 amino acids, about 13 amino acids to about 40 amino acids, about 13 amino acids to about 35 amino acids, about 13 amino acids to about 30 amino acids, about 13 amino acids to about 25 amino acids, about 13 amino acids to about 24 amino acids, about 13 amino acids to about 22 amino acids, about 13 amino acids to about 20 amino acids, about 13 amino acids to about 18 amino acids, about 13 amino acids to about 16 amino acids, about 13 amino acids to about 14 amino acids, about 14 amino acids to about 500 amino acids, about 14 amino acids to about 475 amino acids, about 14 amino acids to about 450 amino acids, about 14 amino acids to about 425 amino acids, about 14 amino acids to about 400 amino acids, about 14 amino acids to about 375 amino acids, about 14 amino acids to about 350 amino acids, about 14 amino acids to about 325 amino acids, about 14 amino acids to about 300 amino acids, about 14 amino acids to about 275 amino acids, about 14 amino acids to about 250 amino acids, about 14 amino acids to about 225 amino acids, about 14 amino acids to about 200 amino acids, about 14 amino acids to about 175 amino acids, about 14 amino acids to about 150 amino acids, about 14 amino acids to about 125 amino acids, about 14 amino acids to about 100 amino acids, about 14 amino acids to about 75 amino acids, about 14 amino acids to about 50 amino acids, about 14 amino acids to about 45 amino acids, about 14 amino acids to about 40 amino acids, about 14 amino acids to about 35 amino acids, about 14 amino acids to about 30 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 15 amino acids to about 500 amino acids, about 15 amino acids to about 475 amino acids, about 15 amino acids to about 450 amino acids, about 15 amino acids to about 425 amino acids, about 15 amino acids to about 400 amino acids, about 15 amino acids to about 375 amino acids, about 15 amino acids to about 350 amino acids, about 15 amino acids to about 325 amino acids, about 15 amino acids to about 300 amino acids, about 15 amino acids to about 275 amino acids, about 15 amino acids to about 250 amino acids, about 15 amino acids to about 225 amino acids, about 15 amino acids to about 200 amino acids, about 15 amino acids to about 175 amino acids, about 15 amino acids to about 150 amino acids, about 15 amino acids to about 125 amino acids, about 15 amino acids to about 100 amino acids, about 15 amino acids to about 75 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 24 amino acids, about 15 amino acids to about 22 amino acids, about 15 amino acids to about 20 amino acids, about 15 amino acids to about 18 amino acids, about 15 amino acids to about 16 amino acids, about 16 amino acids to about 500 amino acids, about 16 amino acids to about 475 amino acids, about 16 amino acids to about 450 amino acids, about 16 amino acids to about 425 amino acids, about 16 amino acids to about 400 amino acids, about 16 amino acids to about 375 amino acids, about 16 amino acids to about 350 amino acids, about 16 amino acids to about 325 amino acids, about 16 amino acids to about 300 amino acids, about 16 amino acids to about 275 amino acids, about 16 amino acids to about 250 amino acids, about 16 amino acids to about 225 amino acids, about 16 amino acids to about 200 amino acids, about 16 amino acids to about 175 amino acids, about 16 amino acids to about 150 amino acids, about 16 amino acids to about 125 amino acids, about 16 amino acids to about 100 amino acids, about 16 amino acids to about 75 amino acids, about 16 amino acids to about 50 amino acids, about 16 amino acids to about 45 amino acids, about 16 amino acids to about 40 amino acids, about 16 amino acids to about 35 amino acids, about 16 amino acids to about 30 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 17 amino acids to about 500 amino acids, about 17 amino acids to about 475 amino acids, about 17 amino acids to about 450 amino acids, about 17 amino acids to about 425 amino acids, about 17 amino acids to about 400 amino acids, about 17 amino acids to about 375 amino acids, about 17 amino acids to about 350 amino acids, about 17 amino acids to about 325 amino acids, about 17 amino acids to about 300 amino acids, about 17 amino acids to about 275 amino acids, about 17 amino acids to about 250 amino acids, about 17 amino acids to about 225 amino acids, about 17 amino acids to about 200 amino acids, about 17 amino acids to about 175 amino acids, about 17 amino acids to about 150 amino acids, about 17 amino acids to about 125 amino acids, about 17 amino acids to about 100 amino acids, about 17 amino acids to about 75 amino acids, about 17 amino acids to about 50 amino acids, about 17 amino acids to about 45 amino acids, about 17 amino acids to about 40 amino acids, about 17 amino acids to about 35 amino acids, about 17 amino acids to about 30 amino acids, about 17 amino acids to about 25 amino acids, about 17 amino acids to about 24 amino acids, about 17 amino acids to about 22 amino acids, about 17 amino acids to about 20 amino acids, about 17 amino acids to about 18 amino acids, about 18 amino acids to about 500 amino acids, about 18 amino acids to about 475 amino acids, about 18 amino acids to about 450 amino acids, about 18 amino acids to about 425 amino acids, about 18 amino acids to about 400 amino acids, about 18 amino acids to about 375 amino acids, about 18 amino acids to about 350 amino acids, about 18 amino acids to about 325 amino acids, about 18 amino acids to about 300 amino acids, about 18 amino acids to about 275 amino acids, about 18 amino acids to about 250 amino acids, about 18 amino acids to about 225 amino acids, about 18 amino acids to about 200 amino acids, about 18 amino acids to about 175 amino acids, about 18 amino acids to about 150 amino acids, about 18 amino acids to about 125 amino acids, about 18 amino acids to about 100 amino acids, about 18 amino acids to about 75 amino acids, about 18 amino acids to about 50 amino acids, about 18 amino acids to about 45 amino acids, about 18 amino acids to about 40 amino acids, about 18 amino acids to about 35 amino acids, about 18 amino acids to about 30 amino acids, about 18 amino acids to about 25 amino acids, about 18 amino acids to about 24 amino acids, about 18 amino acids to about 22 amino acids, about 18 amino acids to about 20 amino acids, about 19 amino acids to about 500 amino acids, about 19 amino acids to about 475 amino acids, about 19 amino acids to about 450 amino acids, about 19 amino acids to about 425 amino acids, about 19 amino acids to about 400 amino acids, about 19 amino acids to about 375 amino acids, about 19 amino acids to about 350 amino acids, about 19 amino acids to about 325 amino acids, about 19 amino acids to about 300 amino acids, about 19 amino acids to about 275 amino acids, about 19 amino acids to about 250 amino acids, about 19 amino acids to about 225 amino acids, about 19 amino acids to about 200 amino acids, about 19 amino acids to about 175 amino acids, about 19 amino acids to about 150 amino acids, about 19 amino acids to about 125 amino acids, about 19 amino acids to about 100 amino acids, about 19 amino acids to about 75 amino acids, about 19 amino acids to about 50 amino acids, about 19 amino acids to about 45 amino acids, about 19 amino acids to about 40 amino acids, about 19 amino acids to about 35 amino acids, about 19 amino acids to about 30 amino acids, about 19 amino acids to about 25 amino acids, about 19 amino acids to about 24 amino acids, about 19 amino acids to about 22 amino acids, about 19 amino acids to about 20 amino acids, about 20 amino acids to about 500 amino acids, about 20 amino acids to about 475 amino acids, about 20 amino acids to about 450 amino acids, about 20 amino acids to about 425 amino acids, about 20 amino acids to about 400 amino acids, about 20 amino acids to about 375 amino acids, about 20 amino acids to about 350 amino acids, about 20 amino acids to about 325 amino acids, about 20 amino acids to about 300 amino acids, about 20 amino acids to about 275 amino acids, about 20 amino acids to about 250 amino acids, about 20 amino acids to about 225 amino acids, about 20 amino acids to about 200 amino acids, about 20 amino acids to about 175 amino acids, about 20 amino acids to about 150 amino acids, about 20 amino acids to about 125 amino acids, about 20 amino acids to about 100 amino acids, about 20 amino acids to about 75 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 20 amino acids to about 24 amino acids, about 21 amino acids to about 22 amino acids, about 21 amino acids to about 500 amino acids, about 21 amino acids to about 475 amino acids, about 21 amino acids to about 450 amino acids, about 21 amino acids to about 425 amino acids, about 21 amino acids to about 400 amino acids, about 21 amino acids to about 375 amino acids, about 21 amino acids to about 350 amino acids, about 21 amino acids to about 325 amino acids, about 21 amino acids to about 300 amino acids, about 21 amino acids to about 275 amino acids, about 21 amino acids to about 250 amino acids, about 21 amino acids to about 225 amino acids, about 21 amino acids to about 200 amino acids, about 21 amino acids to about 175 amino acids, about 21 amino acids to about 150 amino acids, about 21 amino acids to about 125 amino acids, about 15 amino acids to about 100 amino acids, about 21 amino acids to about 75 amino acids, about 21 amino acids to about 50 amino acids, about 21 amino acids to about 45 amino acids, about 21 amino acids to about 40 amino acids, about 21 amino acids to about 35 amino acids, about 21 amino acids to about 30 amino acids, about 21 amino acids to about 25 amino acids, about 21 amino acids to about 24 amino acids, about 21 amino acids to about 22 amino acids, about 22 amino acids to about 500 amino acids, about 22 amino acids to about 475 amino acids, about 22 amino acids to about 450 amino acids, about 22 amino acids to about 425 amino acids, about 22 amino acids to about 400 amino acids, about 22 amino acids to about 375 amino acids, about 22 amino acids to about 350 amino acids, about 22 amino acids to about 325 amino acids, about 22 amino acids to about 300 amino acids, about 22 amino acids to about 275 amino acids, about 22 amino acids to about 250 amino acids, about 22 amino acids to about 225 amino acids, about 22 amino acids to about 200 amino acids, about 22 amino acids to about 175 amino acids, about 22 amino acids to about 150 amino acids, about 22 amino acids to about 125 amino acids, about 22 amino acids to about 100 amino acids, about 22 amino acids to about 75 amino acids, about 22 amino acids to about 50 amino acids, about 22 amino acids to about 45 amino acids, about 22 amino acids to about 40 amino acids, about 22 amino acids to about 35 amino acids, about 22 amino acids to about 30 amino acids, about 22 amino acids to about 25 amino acids, about 22 amino acids to about 24 amino acids, about 23 amino acids to about 500 amino acids, about 23 amino acids to about 475 amino acids, about 23 amino acids to about 450 amino acids, about 23 amino acids to about 425 amino acids, about 23 amino acids to about 400 amino acids, about 23 amino acids to about 375 amino acids, about 23 amino acids to about 350 amino acids, about 23 amino acids to about 325 amino acids, about 23 amino acids to about 300 amino acids, about 23 amino acids to about 275 amino acids, about 23 amino acids to about 250 amino acids, about 23 amino acids to about 225 amino acids, about 23 amino acids to about 200 amino acids, about 23 amino acids to about 175 amino acids, about 23 amino acids to about 150 amino acids, about 23 amino acids to about 125 amino acids, about 23 amino acids to about 100 amino acids, about 23 amino acids to about 75 amino acids, about 23 amino acids to about 50 amino acids, about 23 amino acids to about 45 amino acids, about 23 amino acids to about 40 amino acids, about 23 amino acids to about 35 amino acids, about 23 amino acids to about 30 amino acids, about 23 amino acids to about 25 amino acids, about 23 amino acids to about 24 amino acids, about 25 amino acids to about 500 amino acids, about 25 amino acids to about 475 amino acids, about 25 amino acids to about 450 amino acids, about 25 amino acids to about 425 amino acids, about 25 amino acids to about 400 amino acids, about 25 amino acids to about 375 amino acids, about 25 amino acids to about 350 amino acids, about 25 amino acids to about 325 amino acids, about 25 amino acids to about 300 amino acids, about 25 amino acids to about 275 amino acids, about 25 amino acids to about 250 amino acids, about 25 amino acids to about 225 amino acids, about 25 amino acids to about 200 amino acids, about 25 amino acids to about 175 amino acids, about 25 amino acids to about 150 amino acids, about 25 amino acids to about 125 amino acids, about 25 amino acids to about 100 amino acids, about 25 amino acids to about 75 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 26 amino acids to about 500 amino acids, about 26 amino acids to about 475 amino acids, about 26 amino acids to about 450 amino acids, about 26 amino acids to about 425 amino acids, about 26 amino acids to about 400 amino acids, about 26 amino acids to about 375 amino acids, about 26 amino acids to about 350 amino acids, about 26 amino acids to about 325 amino acids, about 26 amino acids to about 300 amino acids, about 26 amino acids to about 275 amino acids, about 26 amino acids to about 250 amino acids, about 26 amino acids to about 225 amino acids, about 26 amino acids to about 200 amino acids, about 26 amino acids to about 175 amino acids, about 26 amino acids to about 150 amino acids, about 26 amino acids to about 125 amino acids, about 26 amino acids to about 100 amino acids, about 26 amino acids to about 75 amino acids, about 26 amino acids to about 50 amino acids, about 26 amino acids to about 45 amino acids, about 26 amino acids to about 40 amino acids, about 26 amino acids to about 35 amino acids, about 26 amino acids to about 30 amino acids, about 27 amino acids to about 500 amino acids, about 27 amino acids to about 475 amino acids, about 27 amino acids to about 450 amino acids, about 27 amino acids to about 425 amino acids, about 27 amino acids to about 400 amino acids, about 27 amino acids to about 375 amino acids, about 27 amino acids to about 350 amino acids, about 27 amino acids to about 325 amino acids, about 27 amino acids to about 300 amino acids, about 27 amino acids to about 275 amino acids, about 27 amino acids to about 250 amino acids, about 27 amino acids to about 225 amino acids, about 27 amino acids to about 200 amino acids, about 27 amino acids to about 175 amino acids, about 27 amino acids to about 150 amino acids, about 27 amino acids to about 125 amino acids, about 27 amino acids to about 100 amino acids, about 27 amino acids to about 75 amino acids, about 27 amino acids to about 50 amino acids, about 27 amino acids to about 45 amino acids, about 27 amino acids to about 40 amino acids, about 27 amino acids to about 35 amino acids, about 27 amino acids to about 30 amino acids, about 28 amino acids to about 500 amino acids, about 28 amino acids to about 475 amino acids, about 28 amino acids to about 450 amino acids, about 28 amino acids to about 425 amino acids, about 28 amino acids to about 400 amino acids, about 28 amino acids to about 375 amino acids, about 28 amino acids to about 350 amino acids, about 28 amino acids to about 325 amino acids, about 28 amino acids to about 300 amino acids, about 28 amino acids to about 275 amino acids, about 28 amino acids to about 250 amino acids, about 28 amino acids to about 225 amino acids, about 28 amino acids to about 200 amino acids, about 28 amino acids to about 175 amino acids, about 28 amino acids to about 150 amino acids, about 28 amino acids to about 125 amino acids, about 28 amino acids to about 100 amino acids, about 28 amino acids to about 75 amino acids, about 28 amino acids to about 50 amino acids, about 28 amino acids to about 45 amino acids, about 28 amino acids to about 40 amino acids, about 28 amino acids to about 35 amino acids, about 28 amino acids to about 30 amino acids, about 29 amino acids to about 500 amino acids, about 29 amino acids to about 475 amino acids, about 29 amino acids to about 450 amino acids, about 29 amino acids to about 425 amino acids, about 29 amino acids to about 400 amino acids, about 29 amino acids to about 375 amino acids, about 29 amino acids to about 350 amino acids, about 29 amino acids to about 325 amino acids, about 29 amino acids to about 300 amino acids, about 29 amino acids to about 275 amino acids, about 29 amino acids to about 250 amino acids, about 29 amino acids to about 225 amino acids, about 29 amino acids to about 200 amino acids, about 29 amino acids to about 175 amino acids, about 29 amino acids to about 150 amino acids, about 29 amino acids to about 125 amino acids, about 29 amino acids to about 100 amino acids, about 29 amino acids to about 75 amino acids, about 29 amino acids to about 50 amino acids, about 29 amino acids to about 45 amino acids, about 29 amino acids to about 40 amino acids, about 29 amino acids to about 35 amino acids, about 29 amino acids to about 30 amino acids, about 30 amino acids to about 500 amino acids, about 30 amino acids to about 475 amino acids, about 30 amino acids to about 450 amino acids, about 30 amino acids to about 425 amino acids, about 30 amino acids to about 400 amino acids, about 30 amino acids to about 375 amino acids, about 30 amino acids to about 350 amino acids, about 30 amino acids to about 325 amino acids, about 30 amino acids to about 300 amino acids, about 30 amino acids to about 275 amino acids, about 30 amino acids to about 250 amino acids, about 30 amino acids to about 225 amino acids, about 30 amino acids to about 200 amino acids, about 39 amino acids to about 175 amino acids, about 30 amino acids to about 150 amino acids, about 30 amino acids to about 125 amino acids, about 30 amino acids to about 100 amino acids, about 30 amino acids to about 90 amino acids, about 30 amino acids to about 50 amino acids, about 30 amino acids to about 45 amino acids, about 30 amino acids to about 40 amino acids, about 30 amino acids to about 35 amino acids, about 35 amino acids to about 500 amino acids, about 35 amino acids to about 475 amino acids, about 35 amino acids to about 450 amino acids, about 35 amino acids to about 425 amino acids, about 35 amino acids to about 400 amino acids, about 35 amino acids to about 375 amino acids, about 35 amino acids to about 350 amino acids, about 35 amino acids to about 325 amino acids, about 35 amino acids to about 300 amino acids, about 35 amino acids to about 275 amino acids, about 35 amino acids to about 250 amino acids, about 35 amino acids to about 225 amino acids, about 35 amino acids to about 200 amino acids, about 35 amino acids to about 175 amino acids, about 35 amino acids to about 150 amino acids, about 35 amino acids to about 125 amino acids, about 35 amino acids to about 100 amino acids, about 35 amino acids to about 75 amino acids, about 35 amino acids to about 50 amino acids, about 35 amino acids to about 45 amino acids, about 35 amino acids to about 40 amino acids, about 40 amino acids to about 500 amino acids, about 40 amino acids to about 475 amino acids, about 40 amino acids to about 450 amino acids, about 40 amino acids to about 425 amino acids, about 40 amino acids to about 400 amino acids, about 40 amino acids to about 375 amino acids, about 40 amino acids to about 350 amino acids, about 40 amino acids to about 325 amino acids, about 40 amino acids to about 300 amino acids, about 40 amino acids to about 275 amino acids, about 40 amino acids to about 250 amino acids, about 40 amino acids to about 225 amino acids, about 40 amino acids to about 200 amino acids, about 40 amino acids to about 175 amino acids, about 40 amino acids to about 150 amino acids, about 40 amino acids to about 125 amino acids, about 40 amino acids to about 100 amino acids, about 40 amino acids to about 75 amino acids, about 40 amino acids to about 50 amino acids, about 40 amino acids to about 45 amino acids, about 45 amino acids to about 500 amino acids, about 45 amino acids to about 475 amino acids, about 45 amino acids to about 450 amino acids, about 45 amino acids to about 425 amino acids, about 45 amino acids to about 400 amino acids, about 45 amino acids to about 375 amino acids, about 45 amino acids to about 350 amino acids, about 45 amino acids to about 325 amino acids, about 45 amino acids to about 300 amino acids, about 45 amino acids to about 275 amino acids, about 45 amino acids to about 250 amino acids, about 45 amino acids to about 225 amino acids, about 45 amino acids to about 200 amino acids, about 45 amino acids to about 175 amino acids, about 45 amino acids to about 150 amino acids, about 45 amino acids to about 125 amino acids, about 45 amino acids to about 100 amino acids, about 45 amino acids to about 75 amino acids, about 45 amino acids to about 50 amino acids, about 50 amino acids to about 500 amino acids, about 50 amino acids to about 475 amino acids, about 50 amino acids to about 450 amino acids, about 50 amino acids to about 425 amino acids, about 50 amino acids to about 400 amino acids, about 50 amino acids to about 375 amino acids, about 50 amino acids to about 350 amino acids, about 50 amino acids to about 325 amino acids, about 50 amino acids to about 300 amino acids, about 50 amino acids to about 275 amino acids, about 50 amino acids to about 250 amino acids, about 50 amino acids to about 225 amino acids, about 50 amino acids to about 200 amino acids, about 50 amino acids to about 175 amino acids, about 50 amino acids to about 150 amino acids, about 50 amino acids to about 125 amino acids, about 50 amino acids to about 100 amino acids, about 50 amino acids to about 75 amino acids, about 60 amino acids to about 500 amino acids, about 60 amino acids to about 475 amino acids, about 60 amino acids to about 450 amino acids, about 60 amino acids to about 425 amino acids, about 60 amino acids to about 400 amino acids, about 60 amino acids to about 375 amino acids, about 60 amino acids to about 350 amino acids, about 60 amino acids to about 325 amino acids, about 60 amino acids to about 300 amino acids, about 60 amino acids to about 275 amino acids, about 60 amino acids to about 250 amino acids, about 60 amino acids to about 225 amino acids, about 60 amino acids to about 200 amino acids, about 60 amino acids to about 175 amino acids, about 60 amino acids to about 150 amino acids, about 60 amino acids to about 125 amino acids, about 60 amino acids to about 100 amino acids, about 60 amino acids to about 75 amino acids, about 70 amino acids to about 500 amino acids, about 70 amino acids to about 475 amino acids, about 70 amino acids to about 450 amino acids, about 70 amino acids to about 425 amino acids, about 70 amino acids to about 400 amino acids, about 70 amino acids to about 375 amino acids, about 70 amino acids to about 350 amino acids, about 70 amino acids to about 325 amino acids, about 70 amino acids to about 300 amino acids, about 70 amino acids to about 275 amino acids, about 70 amino acids to about 250 amino acids, about 70 amino acids to about 225 amino acids, about 70 amino acids to about 200 amino acids, about 70 amino acids to about 175 amino acids, about 70 amino acids to about 150 amino acids, about 70 amino acids to about 125 amino acids, about 70 amino acids to about 100 amino acids, about 70 amino acids to about 75 amino acids, about 80 amino acids to about 500 amino acids, about 80 amino acids to about 475 amino acids, about 80 amino acids to about 450 amino acids, about 80 amino acids to about 425 amino acids, about 80 amino acids to about 400 amino acids, about 80 amino acids to about 375 amino acids, about 80 amino acids to about 350 amino acids, about 80 amino acids to about 325 amino acids, about 80 amino acids to about 300 amino acids, about 80 amino acids to about 275 amino acids, about 80 amino acids to about 250 amino acids, about 80 amino acids to about 225 amino acids, about 80 amino acids to about 200 amino acids, about 80 amino acids to about 175 amino acids, about 80 amino acids to about 150 amino acids, about 80 amino acids to about 125 amino acids, about 80 amino acids to about 100 amino acids, about 90 amino acids to about 500 amino acids, about 90 amino acids to about 475 amino acids, about 90 amino acids to about 450 amino acids, about 90 amino acids to about 425 amino acids, about 90 amino acids to about 400 amino acids, about 90 amino acids to about 375 amino acids, about 90 amino acids to about 350 amino acids, about 90 amino acids to about 325 amino acids, about 90 amino acids to about 300 amino acids, about 90 amino acids to about 275 amino acids, about 90 amino acids to about 250 amino acids, about 90 amino acids to about 225 amino acids, about 90 amino acids to about 200 amino acids, about 90 amino acids to about 175 amino acids, about 90 amino acids to about 150 amino acids, about 90 amino acids to about 125 amino acids, about 90 amino acids to about 100 amino acids, about 100 amino acids to about 500 amino acids, about 100 amino acids to about 475 amino acids, about 100 amino acids to about 450 amino acids, about 100 amino acids to about 425 amino acids, about 100 amino acids to about 400 amino acids, about 100 amino acids to about 375 amino acids, about 100 amino acids to about 350 amino acids, about 100 amino acids to about 325 amino acids, about 100 amino acids to about 300 amino acids, about 100 amino acids to about 275 amino acids, about 100 amino acids to about 250 amino acids, about 100 amino acids to about 225 amino acids, about 100 amino acids to about 200 amino acids, about 100 amino acids to about 175 amino acids, about 100 amino acids to about 150 amino acids, about 100 amino acids to about 125 amino acids, about 150 amino acids to about 500 amino acids, about 150 amino acids to about 475 amino acids, about 150 amino acids to about 450 amino acids, about 150 amino acids to about 425 amino acids, about 150 amino acids to about 400 amino acids, about 150 amino acids to about 375 amino acids, about 150 amino acids to about 350 amino acids, about 150 amino acids to about 325 amino acids, about 150 amino acids to about 300 amino acids, about 150 amino acids to about 275 amino acids, about 150 amino acids to about 250 amino acids, about 150 amino acids to about 225 amino acids, about 150 amino acids to about 200 amino acids, about 150 amino acids to about 175 amino acids, about 200 amino acids to about 500 amino acids, about 200 amino acids to about 475 amino acids, about 200 amino acids to about 450 amino acids, about 200 amino acids to about 425 amino acids, about 200 amino acids to about 400 amino acids, about 200 amino acids to about 375 amino acids, about 200 amino acids to about 350 amino acids, about 200 amino acids to about 325 amino acids, about 200 amino acids to about 300 amino acids, about 200 amino acids to about 275 amino acids, about 200 amino acids to about 250 amino acids, about 200 amino acids to about 225 amino acids, about 250 amino acids to about 500 amino acids, about 250 amino acids to about 475 amino acids, about 250 amino acids to about 450 amino acids, about 250 amino acids to about 425 amino acids, about 250 amino acids to about 400 amino acids, about 250 amino acids to about 375 amino acids, about 250 amino acids to about 350 amino acids, about 250 amino acids to about 325 amino acids, about 250 amino acids to about 300 amino acids, about 250 amino acids to about 275 amino acids, about 300 amino acids to about 500 amino acids, about 300 amino acids to about 475 amino acids, about 300 amino acids to about 450 amino acids, about 300 amino acids to about 425 amino acids, about 300 amino acids to about 400 amino acids, about 300 amino acids to about 375 amino acids, about 300 amino acids to about 350 amino acids, about 300 amino acids to about 325 amino acids, about 350 amino acids to about 500 amino acids, about 350 amino acids to about 475 amino acids, about 350 amino acids to about 450 amino acids, about 350 amino acids to about 425 amino acids, about 350 amino acids to about 400 amino acids, about 350 amino acids to about 375 amino acids, about 400 amino acids to about 500 amino acids, about 400 amino acids to about 475 amino acids, about 400 amino acids to about 450 amino acids, about 400 amino acids to about 425 amino acids, about 450 amino acids to about 500 amino acids, about 450 amino acids to about 475 amino acids, about 475 amino acids to about 500 amino acids.

In some embodiment, suitable short connector polypeptides for use at the N-terminal end of the linker can be present. Suitable short connector can include, for example, dipeptides such as-Gly-Ser- (GS), -Gly-Ala- (GA) and -Ala-Ser- (AS). Suitable peptide linkers for use at the C-terminal end of the linker include, for example, dipeptides such as -Leu-Gln-(LQ) and -Thr-Gly- (TG).

In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs. In some embodiments, the linker has one or more Gly-Gly-Gly-Ser (GGGS) sequences (SEQ ID NO: 59), e.g., 1, 2, 3, 4, 5, 6, 7, 8, 9, or 10 or more GGGS sequences. In some embodiments, the linker comprises or consists of Gly-Gly-Gly-Ser (GGGS) sequences (SEQ ID NO: 59). In some embodiments, the linker has one or more Gly-Gly-Gly-Gly-Ser (GGGGS) sequences (SEQ ID NO: 60), e.g., 1, 2, 3, 4, 5, 6, 7, 8, 9, or 10 or more GGGGS sequences. In some embodiments, the linker comprises or consists of Gly-Gly-Gly-Gly-Ser (GGGGS) sequences (SEQ ID NO: 60). In some embodiments, the linker has one or more Gly-Gly-Ser-Gly (GGSG) sequences (SEQ ID NO: 61), e.g., 1, 2, 3, 4, 5, 6, 7, 8, 9, or 10 or more GGSG sequences. In some embodiments, the linker comprises or consists of Gly-Gly-Ser-Gly (GGSG) sequences (SEQ ID NO: 61). In some embodiments, the linker has one or more -Gly-Ser-Gly-Gly-Gly-Ser-Gly (SEQ ID NO: 23), e.g., 1, 2, 3, 4, 5, 6, 7, 8, 9, or 10 or more GSGGGSG sequences. In some embodiments, the linker comprises or consists of amino acids -Gly-Ser-Gly-Gly-Gly-Ser-Gly (SEQ ID NO: 23).

In some embodiments, the peptide linker (e.g. linker comprising or consisting of one or more -Gly-Ser-Gly-Gly-Gly-Ser-Gly, SEQ ID NO: 23) links the activator domain at the N-terminus to the half-life modulator (e.g. HSA or variant thereof). Yet, in other embodiments, the peptide linker (e.g. linker comprising or consisting of one or more -Gly-Ser-Gly-Gly-Gly-Ser-Gly, SEQ ID NO: 23) links the targeting domain at the N-terminus to the half-life modulator.

In some embodiments, the peptide linker that links the activator domain (e.g., IGF-1 or variant thereof) at the N-terminus to the half-life modulator (e.g., HSA or variant thereof) is a flexible linker (e.g. linker rich in glycine and/or serine residues).

In some embodiments, the peptide linker that links the targeting domain (e.g., single chain variable fragment, scFv) at the C-terminus to the half-life modulator (e.g., HSA or variant thereof) is a non-flexible linker. In some embodiments, the non-flexible linker separates the targeting domain from the half-life modulator by incorporating a structured domain of about 30 Å long to about 120 Å long. For example, the non-flexible linker can configured to allow separation of the targeting domain from the half-life modulator by about 30 Å to about 120 A, about 30 Å to about 110 Å, about 30 Å to about 100 Å, about 30 Å to about 90 Å, about 30 Å to about 80 Å, about 30 Å to about 70 Å, about 30 Å to about 60 Å, about 30 Å to about 50 Å, about 30 Å to about 40 Å, about 40 Å to about 120 Å, about 40 Å to about 110 Å, about 40 Å to about 100 Å, about 40 Å to about 90 Å, about 40 Å to about 80 Å, about 40 Å to about 70 Å, about 40 Å to about 60 Å, about 40 Å to about 50 Å, about 50 Å to about 120 Å, about 50 Å to about 110 Å, about 50 Å to about 100 Å, about 50 Å to about 90 Å, about 50 Å to about 80 Å, about 50 Å to about 70 Å, about 50 Å to about 60 Å, about 60 Å to about 120 Å, about 60 Å to about 110 Å, about 60 Å to about 100 Å, about 60 Å to about 90 A, about 60 Å to about 80 Å, about 60 Å to about 70 Å, about 70 Å to about 120 Å, about 70 Å to about 110 Å, about 70 Å to about 100 Å, about 70 Å to about 90 Å, about 70 Å to about 80 Å, about 80 Å to about 120 Å, about 80 Å to about 110 Å, about 80 Å to about 100 Å, about 80 Å to about 90 Å, about 90 Å to about 120 Å, about 90 Å to about 110 Å, about 90 Å to about 100 Å, about 100 Å to about 120 Å, about 100 Å to about 110 Å, about 110 Å to about 120 Å, or about 30 Å, about 40 Å, about 50 Å, about 60 Å, about 70 Å, about 90 Å, about 100 Å, about 110 Å, about 120 Å

In some embodiments, the structured peptide linkers effectively limit the intra-domain interactions (half-life modulator to targeting domain of the fusion proteins), thereby decoupling the kinetics of folding to improve expression and purification of the fusion proteins described herein.

In some embodiments, the structured peptide linker that separates the half-life modulator from the targeting domain of the fusion proteins promotes the formation of the native disulfide linkages in each domain. In some embodiments, the structured peptide linker that separates the half-life modulator from the targeting domain of the fusion proteins limits the formation of the disulfide linkages between the targeting domain and the half-life modulator.

In some embodiments, the structured peptide linkers separate the targeting domain from the half-life modulator, resulting in less steric hindrance, thereby enabling independent function of the two domains, improving half-life modulator and/or targeting activity.

In some embodiments, the structured peptide linker comprise or consists of a tetratricopeptide repeat (TPR) domain. TPR domains are alpha helix rich. A TPR domain consists of multiple (two or more) repeats of 34 amino acids sharing a degenerate consensus sequence defined by a pattern of small and large hydrophobic amino acids. The TPR domain provides a rigid linker and limit interaction between the half-life modulator (e.g. human serum albumin or variant thereof) and the targeting domain (e.g. scFv). In some embodiments, the sequence of the TPR comprises or consists of KLSGGGGSGGGGSGGGGSAEAWYNLGNAYYKQGDYQKAIEYYQKALELDPNNAE AWYNLGNAYYKQGDYQKAIEYYQKALELDPNNAEAWYNLGNAYYKQGDYQKAIE DYQKALELDPNNLQRSAGGGGSGGGGSGGGGAS (SEQ ID NO: 24).

In some embodiments, the structured peptide linker comprising TPR links the C-terminus of the half-life modulator and the N-terminus of the targeting domain. In other embodiments, the structured peptide linker comprising TPR links the N-terminus of the half-life modulator and the C-terminus of the targeting domain.

In some embodiments, the structured peptide linker comprises or consist of an amino acid having the sequence of cTPR3, cTPR6, cTPR9, cTPR12 or any combination of the foregoing. In some embodiments, the structured linker has an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NOs: 24, 51-54. In some embodiments, the structured linker has an amino acid sequence set forth in SEQ ID NOs; 24, 51-54 as shown below:

SEQ ID NO: 51 is the amino acid sequence of linker
cTPR3
KLSGGGGSGGGGSGGGGSAEAWYNLGNAYYKQGDYQKAIEYYQKALELDP
NNAEAWYNLGNAYYKQGDYQKAIEYYQKALELDPNNAEAWYNLGNAYYKQ
GDYQKAIEDYQKALELDPNNLQRSAGGGGSGGGGSGGGGAS
SEQ ID NO: 52 is the amino acid sequence of linker
cTPR6
KLSGGGGSGGGGSGGGGSAEAWYNLGNAYYKQGDYQKAIEYYQKALELDP
NNAEAWYNLGNAYYKQGDYQKAIEYYQKALELDPNNAEAWYNLGNAYYKQ
GDYQKAIEDYQKALELDPNNLQAEAWKNLGNAYYKQGDYQKAIEYYQKAL
ELDPNNASAWYNLGNAYYKQGDYQKAIEYYQKALELDPNNAKAWYRRGNA
YYKQGDYQKAIEDYQKALELDPNNRSRSAGGGGSGGGGSGGGGAS
SEQ ID NO: 53 is the amino acid sequence of linker
cTPR9
KLSGGGGSGGGGSGGGGSAEAWYNLGNAYYKQGDYQKAIEYYQKALELDP
NNAEAWYNLGNAYYKQGDYQKAIEYYQKALELDPNNAEAWYNLGNAYYKQ
GDYQKAIEDYQKALELDPNNLQAEAWKNLGNAYYKQGDYQKAIEYYQKAL
ELDPNNASAWYNLGNAYYKQGDYQKAIEYYQKALELDPNNAKAWYRRGNA
YYKQGDYQKAIEDYQKALELDPNNRSAEAWYNLGNAYYKQGDYQKAIEYY
QKALELDPNNAEAWYNLGNAYYKQGDYQKAIEYYQKALELDPNNAEAWYN
LGNAYYKQGDYQKAIEDYQKALELDPNNLQRSAGGGGSGGGGSGGGGAS
SEQ ID NO: 54 is the amino acid sequence of linker
cTPR12
KLSGGGGSGGGGSGGGGSAEAWYNLGNAYYKQGDYQKAIEYYQKALELDP
NNAEAWYNLGNAYYKQGDYQKAIEYYQKALELDPNNAEAWYNLGNAYYKQ
GDYQKAIEDYQKALELDPNNLQAEAWKNLGNAYYKQGDYQKAIEYYQKAL
ELDPNNASAWYNLGNAYYKQGDYQKAIEYYQKALELDPNNAKAWYRRGNA
YYKQGDYQKAIEDYQKALELDPNNRSAEAWYNLGNAYYKQGDYQKAIEYY
QKALELDPNNAEAWYNLGNAYYKQGDYQKAIEYYQKALELDPNNAEAWYN
LGNAYYKQGDYQKAIEDYQKALELDPNNLQAEAWKNLGNAYNAYYKQGDY
YKQGDYQKAIEYYQKALELDPNNASAWYNLGQKAIEYYQKALELDPNNAK
AWYRRGNAYYKQGDYQKAIEDYQKALELDPNNRS
AGGGGSGGGGSGGGGAS

### Polypeptide Domain

Provided herein are polypeptide domains that separate the ScFv targeting domain and the activator domain of the chimeric proteins described herein. In some embodiments, the polypeptide domain comprises the half-life modulator (also referred herein as scaffold domain) with a first peptide linker at its N-terminus and a second peptide linker at its C-terminus. In some embodiments, the polypeptide domain comprises human serum albumin or variant thereof with a first peptide linker at its N-terminus and a second peptide linker at its C-terminus. In some embodiments, the polypeptide domain comprises human serum albumin or variant thereof with a first peptide linker at its N-terminus and a second peptide linker at its C-terminus. In some embodiments, the polypeptide domain comprises human serum albumin or variant thereof with a first flexible peptide linker at its N-terminus and a second non-flexible peptide linker at its C-terminus. (See FIG. 1C). Yet in other embodiments, the polypeptide domain comprises human serum albumin or variant thereof with a first non-flexible peptide linker at its N-terminus and a second flexible peptide linker at its C-terminus. (See FIG. 1D).

In some embodiments, the peptide linker is present at the N-terminus, at the C-terminus or at both the N-terminus and the C-terminus of the half-life modulator. In some embodiments, the chimeric protein comprises a half-life modulator comprising a first peptide linker at the N-terminus of the half-life modulator and a second peptide linker at the C-terminus of the half-life modulator. In some embodiments, the chimeric protein comprises a polypeptide domain comprising a first peptide linker at the N-terminus of a human serum albumin or variant thereof and a second peptide linker at the C-terminus of the human serum albumin or variant thereof. In some embodiments, the variant of human serum albumin consists of or comprises SEQ ID NO: 15. In some embodiments, the variant of human serum albumin consists of or comprises of an amino acid sequences having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 15. In some embodiments, the variant of human serum albumin consists of or comprises of an amino acid sequences having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 13. In some embodiments, the variant of human serum albumin consists of or comprises of an amino acid sequences having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 14. In some embodiments, the variant of human serum albumin consists of or comprises of an amino acid sequences having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 12.

In some embodiments, the flexible peptide linker can be a glycine rich peptide linker (e.g. peptide linker having an amino acid sequence as set forth in Seq ID NO: 23, SEQ ID NO: 49, SEQ ID NO: 50, SEQ ID NO; 59, SEQ ID NO; 60, Seq ID NO: 61).

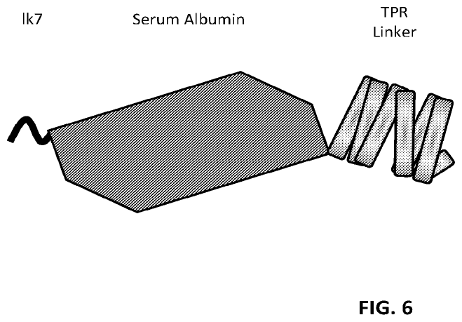

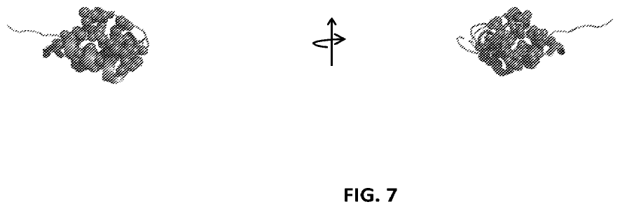

In some embodiments, the flexible peptide linker comprises or consists of one to seven repeats of 1k7 ((GSGGGSG)i -7). In some embodiments, the non-flexible peptide linker comprises or consists of a tetratricopeptide repeat (TPR) domain. In some embodiments, the amino acid sequence of the polypeptide domain comprises or consists of 1k7 ((GSGGGSG)₁₋₇), HSA or variant thereof, consensus tetra-trico-peptide (TPR) repeat domains described herein. See FIG. 1C, FIG. 1D and FIG. 6. Without being bound to the theory, such polypeptide domains allow for easy purification of the resulting chimeric proteins (see Example 3). In some embodiments, the polypeptide domain can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 55, SEQ ID NO: 56, SEQ ID NO: 57, or SEQ ID NO: 58. In some embodiments, the scaffold domain can have an amino acid sequence set forth in SEQ ID NO: 55, SEQ ID NO: 56, SEQ ID NO: 57, or SEQ ID NO: 58. In some embodiments, these linkers link the targeting domain and the activator domain of the chimeric protein.

SEQ ID NO: 55 is the amino acid sequence of lk7-Human Serum Albumin-
CTPR3.
GSGGGSGDAHKSEVAHRFKDLGEENFKALVLIAFAQYLQQSPFEDHVKLVNEVTEFAKTCVA
DESAENCDKSLHTLFGDKLCTVATLRETYGEMADCCAKQEPERNECFLQHKDDNPNLPRLVR
PEVDVMCTAFHDNEETFLKKYLYEIARRHPYFYAPELLFFAKRYKAAFTECCQAADKAACLL
PKLDELRDEGKASSAKQRLKCASLQKFGERAFKAWAVARLSQRFPKAEFAEVSKLVTDLTKV
HTECCHGDLLECADDRADLAKYICENQDSISSKLKECCEKPLLEKSHCIAEVENDEMPADLP
SLAADFVESKDVCKNYAEAKDVFLGMFLYEYARRHPDYSVVLLLRLAKTYETTLEKCCAAAD
PHECYAKVFDEFKPLVEEPQNLIKQNCELFEQLGEYKFQNALLVRYTKKVPQVSTPTLVEVS
RNLGKVGSKCCKHPEAKRMPCAEDYLSVVLNQLCVLHEKTPVSDRVTKCCTESLVNRRPCES
ALEVDETYVPKEFQAETFTFHADICTLSEKERQIKKQTALVELVKHKPKATKEQLKAVMDDE
AAFVEKCCKADDKETCFAEEGKKLVAASQAALGLKLSGGGGSGGGGSGGGGSAEAWYNLGNA
YYKQGDYQKAIEYYQKALELDPNNAEAWYNLGNAYYKQGDYQKAIEYYQKALELDPNNAEAW
YNLGNAYYKQGDYQKAIEDYQKALELDPNNLQRSAGGGGSGGGGGGGGAS
SEQ ID NO: 56 is the amino acid sequence of lk7-Human Serum Albumin-
cTPR6.
GSGGGSGDAHKSEVAHRFKDLGEENFKALVLIAFAQYLQQSPFEDHVKLVNEVTEFAKTCVA
DESAENCDKSLHTLFGDKLCTVATLRETYGEMADCCAKQEPERNECFLQHKDDNPNLPRLVR
PEVDVMCTAFHDNEETFLKKYLYEIARRHPYFYAPELLFFAKRYKAAFTECCQAADKAACLL
PKLDELRDEGKASSAKQRLKCASLQKFGERAFKAWAVARLSQRFPKAEFAEVSKLVTDLTKV
HTECCHGDLLECADDRADLAKYICENQDSISSKLKECCEKPLLEKSHCIAEVENDEMPADLP
SLAADFVESKDVCKNYAEAKDVFLGMFLYEYARRHPDYSVVLLLRLAKTYETTLEKCCAAAD
PHECYAKVFDEFKPLVEEPQNLIKQNCELFEQLGEYKFQNALLVRYTKKVPQVSTPTIVEVS
RNLGKVGSKCCKHPEAKRMPCAEDYLSVVLNQLCVLHEKTPVSDRVTKCCTESLVNRRPCES
ALEVDETYVPKEFQAETFTFHADICTLSEKERQIKKQTALVELVKHKPKATKEQLKAVMDDF
AAFVEKCCKADDKETCFAEEGKKLVAASQAALGLKLSGGGGSGGGGSGGGGSAEAWYNLGNA
YYKQGDYQKAIEYYQKALELDPNNAEAWYNLGNAYYKQGDYQKAIEYYQKALELDPNNAEAW
YNLGNAYYKQGDYQKAIEDYQKALELDPNNLQAEAWKNLGNAYYKQGDYQKAIEYYQKALEL
DPNNASAWYNLGNAYYKQGDYQKAIEYYQKALELDPNNAKAWYRRGNAYYKQGDYQKAIEDY
QKALELDPNNRSRSAGGGGSGGGGSGGGGAS
SEQ ID NO: 57 is the amino acid sequence of lk7-Human Serum Albumin-
cTPR9.
GSGGGSGDAHKSEVAHRFKDLGEENFKALVLIAFAQYLQQSPFEDHVKLVNEVTEFAKTCVA
DESAENCDKSLHTLFGDKLCTVATLRETYGEMADCCAKQEPERNECFLQHKDDNPNLPRLVR
PEVDVMCTAFHDNEETFLKKYLYEIARRHPYFYAPELLFFAKRYKAAFTECCQAADKAACLL
PKLDELRDEGKASSAKQRLKCASLQKFGERAFKAWAVARLSQRFPKAEFAEVSKLVTDLTKV
HTECCHGDLLECADDRADLAKYICENQDSISSKLKECCEKPLLEKSHCIAEVENDEMPADLP
SLAADFVESKDVCKNYAEAKDVFLGMFLYEYARRHPDYSVVLLLRLAKTYETTLEKCCAAAD
PHECYAKVFDEFKPLVEEPQNLIKQNCELFEQLGEYKFQNALLVRYTKKVPQVSTPTLVEVS
RNLGKVGSKCCKHPEAKRMPCAEDYLSVVLNQLCVLHEKTPVSDRVTKCCTESLVNRRPCES
ALEVDETYVPKEFQAETFTFHADICTLSEKERQIKKQTALVELVKHKPKATKEQLKAVMDDF
AAFVEKCCKADDKETCFAEEGKKLVAASQAALGLGSGGGSGDAHKSEVAHRFKDLGEENFKA
LVLIAFAQYLQQSPFEDHVKLVNEVTEFAKTCVADESAENCDKSLHTLFGDKLCTVATLRET
YGEMADCCAKQEPERNECFLQHKDDNPNLPRLVRPEVDVMCTAFHDNEETFLKKYLYEIARR
HPYFYAPELLFFAKRYKAAFTECCQAADKAACLLPKLDELRDEGKASSAKQRLKCASLQKFG
ERAFKAWAVARLSQRFPKAEFAEVSKLVTDLTKVHTECCHGDLLECADDRADLAKYICENQD
SISSKLKECCEKPLLEKSHCIAEVENDEMPADLPSLAADFVESKDVCKNYAEAKDVFLGMEL
YEYARRHPDYSVVLLLRLAKTYETTLEKCCAAADPHECYAKVFDEFKPLVEEPQNLIKQNCE
LFEQLGEYKFQNALLVRYTKKVPQVSTPTLVEVSRNLGKVGSKCCKHPEAKRMPCAEDYLSV
VLNQLCVLHEKTPVSDRVTKCCTESLVNRRPCFSALEVDETYVPKEFQAETFTFHADICTLS
EKERQIKKQTALVELVKHKPKATKEQLKAVMDDFAAFVEKCCKADDKETCFAEEGKKLVAAS
QAALGLKLSGGGGSGGGGSGGGGSAEAWYNLGNAYYKQGDYQKAIEYYQKALELDPNNAEAW
YNLGNAYYKQGDYQKAIEYYQKALELDPNNAEAWYNLGNAYYKQGDYQKAIEDYQKALELDP
NNLQAEAWKNLGNAYYKQGDYQKAIEYYQKALELDPNNASAWYNLGNAYYKQGDYQKAIEYY
QKALELDPNNAKAWYRRGNAYYKQGDYQKAIEDYQKALELDPNNRSAEAWYNLGNAYYKQGD
YQKAIEYYQKALELDPNNAEAWYNLGNAYYKQGDYQKAIEYYQKALELDPNNAEAWYNLGNA
YYKQGDYQKAIEDYQKALELDPNNLQRSAGGGGSGGGGSGGGGAS
SEQ ID NO: 58 is the amino acid sequence of lk7-Human Serum Albumin-
CTPR12.
GSGGGSGDAHKSEVAHRFKDLGEENFKALVLIAFAQYLQQSPFEDHVKLVNEVTEFAKTCVA
DESAENCDKSLHTLFGDKLCTVATLRETYGEMADCCAKQEPERNECFLQHKDDNPNLPRLVR
PEVDVMCTAFHDNEETFLKKYLYEIARRHPYFYAPELLFFAKRYKAAFTECCQAADKAACLL
PKLDELRDEGKASSAKQRLKCASLQKFGERAFKAWAVARLSQRFPKAEFAEVSKLVTDLTKV
HTECCHGDLLECADDRADLAKYICENQDSISSKLKECCEKPLLEKSHCIAEVENDEMPADLP
SLAADFVESKDVCKNYAEAKDVELGMELYEYARRHPDYSVVLLLRLAKTYETTLEKCCAAAD
PHECYAKVFDEFKPLVEEPQNLIKQNCELFEQLGEYKFQNALLVRYTKKVPQVSTPTLVEVS
RNLGKVGSKCCKHPEAKRMPCAEDYLSVVLNQLCVLHEKTPVSDRVTKCCTESLVNRRPCES
ALEVDETYVPKEFQAETFTFHADICTLSEKERQIKKQTALVELVKHKPKATKEQLKAVMDDF
AAFVEKCCKADDKETCFAEEGKKLVAASQAALGLKLSGGGGSGGGGSGGGGSAEAWYNLGNA
YYKQGDYQKAIEYYQKALELDPNNAEAWYNLGNAYYKQGDYQKAIEYYQKALELDPNNAEAW
YNLGNAYYKQGDYQKAIEDYQKALELDPNNLQAEAWKNLGNAYYKQGDYQKAIEYYQKALEL
DPNNASAWYNLGNAYYKQGDYQKAIEYYQKALELDPNNAKAWYRRGNAYYKQGDYQKAIEDY
QKALELDPNNRSAEAWYNLGNAYYKQGDYQKAIEYYQKALELDPNNAEAWYNLGNAYYKQGD
YQKAIEYYQKALELDPNNAEAWYNLGNAYYKQGDYQKAIEDYQKALELDPNNLQAEAWKNLG
NAYYKQGDYQKAIEYYQKALELDPNNASAWYNLGNAYYKQGDYQKAIEYYQKALELDPNNAK
AWYRRGNAYYKQGDYQKAIEDYQKALELDPNNRSAGGGGSGGGGSGGGGAS

### Exemplary Chimeric Proteins

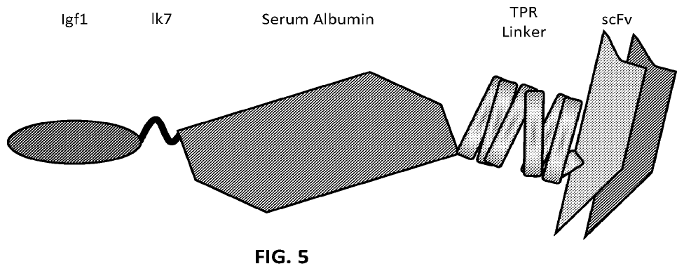

Provided herein are chimeric proteins having a ScFv targeting domain, a polypeptide domain, and an activator domain of the chimeric proteins. See for example FIG. 5. The chimeric proteins of the disclosure, in some embodiments, comprise from the N-terminus to the C-terminus an activator domain comprising a growth factor (including but not limited to IGF-1, IGF-2, Neuregulin1α, Neuregulin1β, BMP2, BMP7, FGF2, FGF9 VEGF-A or variant thereof or fragment thereof), a flexible peptide linker, a half-life modulator (including, but not limited to human serum albumin, or variant thereof, or fragment thereof)), a non-flexible peptide linker, targeting domain comprising a scFv having a binding specificity to a cell surface protein of a kidney tissue (including but not limited Protein Tyrosine Phosphatase Receptor Type O (PTPRO), Podocin, Phospholipase A2 Receptor 1, Integrin Subunit alpha 8, Apoptosis resistant E3 ubiquitin protein ligase 1, Integrin Subunit alpha 8, Apoptosis resistant E3 ubiquitin protein ligase 1, Podocalyxin, Synaptopodin, Alpha 3 integrin, Cysteine rich transmembrane BMP regulator 1, FAT atypical cadherin 1, NEPH1, Dystroglycan, or Podoplanin), a flexible peptide linker, a half-life modulator (including, but not limited to human serum albumin, or variant thereof, or fragment thereof). In other embodiments, the chimeric proteins of the disclosure, comprise from the N-terminus to the C-terminus a targeting domain comprising a scFv having a binding specificity to a cell surface protein of a kidney tissue (including but not limited Protein Tyrosine Phosphatase Receptor Type O (PTPRO), Podocin, Phospholipase A2 Receptor 1, Integrin Subunit alpha 8, Apoptosis resistant E3 ubiquitin protein ligase 1, Integrin Subunit alpha 8, Apoptosis resistant E3 ubiquitin protein ligase 1, Podocalyxin, Synaptopodin, Alpha 3 integrin, Cysteine rich transmembrane BMP regulator 1, FAT atypical cadherin 1, NEPH1, Dystroglycan, or Podoplanin), a non-flexible peptide linker, a half-life modulator (including, but not limited to human serum albumin, or variant thereof, or fragment thereof), a flexible peptide linker, and an activator domain comprising a growth factor (including but not limited to IGF-1, IGF-2, Neuregulin1α, Neuregulin1β, BMP2, BMP7, FGF2, FGF9 VEGF-A or variant thereof or fragment thereof).

In some embodiments, the chimeric proteins comprise the polypeptide domain can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 55, SEQ ID NO: 56, SEQ ID NO: 57, or SEQ ID NO: 58. In some embodiments, the scaffold domain can have an amino acid sequence set forth in SEQ ID NO: 55, SEQ ID NO: 56, SEQ ID NO: 57, or SEQ ID NO: 58.

In some embodiments, the chimeric proteins have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in SEQ ID NO: 41, SEQ ID NO: 42, SEQ ID NO: 43, SEQ ID NO: 45, or SEQ ID NO: 46. In some embodiments, the chimeric proteins have an amino acid sequence set forth in SEQ ID NO: 41, SEQ ID NO: 42, SEQ ID NO: 43, SEQ ID NO: 45, or SEQ ID NO: 46.

In some embodiments, the targeted chimeric proteins described herein exhibit increased targeting to the glomeruli when compared to a similar protein without a scFv targeting domain (non-targeted chimeric protein). In some embodiments, the chimeric proteins exhibit increased targeting to the glomeruli by at least about 10%, at least about 25%., at least about 50% at least about 75%, at least about 100%, at least about 150%, at least about 200%, at least about 300%, at least about 400%, at least about 500%, at least about 600%, at least about 700%, at least about 800%, at least about 900%, at least about 1,000% when compared to a similar protein without a scFv targeting domain.

In some embodiments, the chimeric proteins exhibit increased tissue half life (time period duration after administration, during which the chimeric protein is detectable) by immunohistochemical methods or other methods that can assess the presence of the chimeric proteins. In some embodiments, the chimeric proteins exhibit extended tissue-half in the glomeruli that is at least about 10%, at least about 25%, at least about 50% at least about 75%, at least about 100%, at least about 150%, at least about 200%, at least about 300%, at least about 400%, at least about 500%, at least about 600%, at least about 700%, at least about 800%, at least about 900%, at least about 1,000% when compared to a similar protein without a scFv targeting domain.

It will be apparent that elements in addition to those described above may optionally be included in the proteins provided herein. Such elements may be present for a variety of purposes, including to facilitate expression, preparation or purification of the chimeric protein, or to perform targeting functions.

In some embodiments, the chimeric protein comprises a scFv having a binding specificity to Protein Tyrosine Phosphatase Receptor Type O, and a variant of human insulin-like growth factor IGF-1, wherein the variant of IGF-1 comprises one or more mutations, wherein the one or more mutations consist of a substitution at one or more positions corresponding to E3, Y24, Y31, Y60, and combinations thereof, wherein the scFv is at the C-terminus and the variant of human insulin-like growth factor IGF-1 is at the N-terminus of the chimeric protein.

In some embodiments, the chimeric protein comprises (a) a scFv having a binding specificity to Protein Tyrosine Phosphatase Receptor Type O, (b) a variant of human insulin-like growth factor IGF-1, wherein the variant of IGF-1 comprises one or more mutations, wherein the one or more mutations comprise or consist of a substitution at one or more positions corresponding to E3, Y24, Y31, Y60, and combinations thereof, and (c) a variant of human serum albumin (HSA), wherein the variant of HSA comprises one or more mutations, wherein the one or more mutations comprise or consist of a substitution at one or more positions corresponding to C58 and N527, and combinations thereof, wherein the scFv is at the C-terminus and the variant of human insulin-like growth factor IGF-1 is at the N-terminus of the chimeric protein.

In some embodiments, the chimeric protein comprises (a) a variant of human insulin-like growth factor IGF-1, wherein the variant of IGF-1 comprises one or more mutations, wherein the one or more mutations comprise or consist of a substitution at one or more positions corresponding to E3, Y24, Y31, Y60, and combinations thereof at the N-terminus of the chimeric protein, (b) a first peptide linker at the C-terminus of the variant of human insulin-like growth factor IGF-1, (c) a variant of human serum albumin (HSA) at the C-terminus of the first peptide linker, wherein the variant of HSA comprises one or more mutations, wherein the one or more mutations comprise or consist of a substitution at one or more positions corresponding to C58 and N527, and combinations thereof, (d) a second peptide linker at the C-terminus of the variant of human serum albumin, and (e) a scFv having a binding specificity to Protein Tyrosine Phosphatase Receptor Type O is at the C-terminus of the chimeric protein.

In some embodiments, the peptide first linker comprises or consists of amino acid sequence rich in glycine and serine residues, including but not limited to peptide linker having an amino acid sequence as set forth in Seq ID NO: 23, SEQ ID NO: 49, SEQ ID NO: 50, SEQ ID NO; 59, SEQ ID NO; 60, SEQ ID NO: 61.

In some embodiments, the first linker comprises or consists of amino acid sequence rich in glycine and serine residues and the second linker comprises or consists of a tetratricopeptide repeat (TPR) domain.

In some embodiments, the first linker comprises or consists of amino acid sequence 1 to 7 repeats of (GSGGGSG) (SEQ ID NO: 23) and the second linker comprises or consists of a tetratricopeptide repeat (TPR) domain. In some embodiments, the second linker comprises or consists of amino acids set forth in SEQ ID NO: 24, SEQ ID NO: 51, SEQ ID NO: 52, SEQ ID NO: 53 or SEQ ID NO: 54.

SEQ ID NO: 51 is the amino acid sequence of linker
cTPR3
KLSGGGGSGGGGSGGGGSAEAWYNLGNAYYKQGDYQKAIEYYQKALELDP
NNAEAWYNLGNAYYKQGDYQKAIEYYQKALELDPNNAEAWYNLGNAYYKQ
GDYQKAIEDYQKALELDPNNLQRSAGGGGSGGGGSGGGGAS
SEQ ID NO: 52 is the amino acid sequence of linker
cTPR6
KLSGGGGSGGGGSGGGGSAEAWYNLGNAYYKQGDYQKAIEYYQKALELDP
NNAEAWYNLGNAYYKQGDYQKAIEYYQKALELDPNNAEAWYNLGNAYYKQ
GDYQKAIEDYQKALELDPNNLQAEAWKNLGNAYYKQGDYQKAIEYYQKAL
ELDPNNASAWYNLGNAYYKQGDYQKAIEYYQKALELDPNNAKAWYRRGNA
YYKQGDYQKAIEDYQKALELDPNNRSRSAGGGGSGGGGSGGGGAS
SEQ ID NO: 53 is the amino acid sequence of linker
cTPR9
KLSGGGGSGGGGSGGGGSAEAWYNLGNAYYKQGDYQKAIEYYQKALELDP
NNAEAWYNLGNAYYKQGDYQKAIEYYQKALELDPNNAEAWYNLGNAYYKQ
GDYQKAIEDYQKALELDPNNLQAEAWKNLGNAYYKQGDYQKAIEYYQKAL
ELDPNNASAWYNLGNAYYKQGDYQKAIEYYQKALELDPNNAKAWYRRGNA
YYKQGDYQKAIEDYQKALELDPNNRSAEAWYNLGNAYYKQGDYQKAIEYY
QKALELDPNNAEAWYNLGNAYYKQGDYQKAIEYYQKALELDPNNAEAWYN
LGNAYYKQGDYQKAIEDYQKALELDPNNLQRSAGGGGSGGGGSGGGGAS
SEQ ID NO: 54 is the amino acid sequence of linker
cTPR12
KLSGGGGSGGGGSGGGGSAEAWYNLGNAYYKQGDYQKAIEYYQKALELDP
NNAEAWYNLGNAYYKQGDYQKAIEYYQKALELDPNNAEAWYNLGNAYYKQ
GDYQKAIEDYQKALELDPNNLQAEAWKNLGNAYYKQGDYQKAIEYYQKAL
ELDPNNASAWYNLGNAYYKQGDYQKAIEYYQKALELDPNNAKAWYRRGNA
YYKQGDYQKAIEDYQKALELDPNNRSAEAWYNLGNAYYKQGDYQKAIEYY
QKALELDPNNAEAWYNLGNAYYKQGDYQKAIEYYQKALELDPNNAEAWYN
LGNAYYKQGDYQKAIEDYQKALELDPNNLQAEAWKNLGNAYYKQGDYQKA
IEYYQKALELDPNNASAWYNLGNAYYKQGDYQKAIEYYQKALELDPNNAK
AWYRRGNAYYKQGDYQKAIEDYQKALELDPNNRSAGGGGSGGGGSGGGGA
S

In some embodiments, the chimeric protein comprises a scFv having a binding specificity to nephrin (NEPH1), and a variant of human insulin-like growth factor IGF-1, wherein the variant of IGF-1 comprises one or more mutations, wherein the one or more mutations consist of a substitution at one or more positions corresponding to E3, Y24, Y31, Y60, and combinations thereof, wherein the scFv is at the C-terminus and the variant of human insulin-like growth factor IGF-1 is at the N-terminus of the chimeric protein.

In some embodiments, the chimeric protein comprises (a) a scFv having a binding specificity to nephrin, (b) a variant of human insulin-like growth factor IGF-1, wherein the variant of IGF-1 comprises one or more mutations, wherein the one or more mutations comprise or consist of a substitution at one or more positions corresponding to E3, Y24, Y31, Y60, and combinations thereof, and (c) a variant of human serum albumin (HSA), wherein the variant of HSA comprises one or more mutations, wherein the one or more mutations comprise or consist of a substitution at one or more positions corresponding to C58 and N527, and combinations thereof, wherein the scFv is at the C-terminus and the variant of human insulin-like growth factor IGF-1 is at the N-terminus of the chimeric protein.

In some embodiments, the chimeric protein comprises (a) a variant of human insulin-like growth factor IGF-1, wherein the variant of IGF-1 comprises one or more mutations, wherein the one or more mutations comprise or consist of a substitution at one or more positions corresponding to E3, Y24, Y31, Y60, and combinations thereof at the N-terminus of the chimeric protein, (b) a first peptide linker at the C-terminus of the variant of human insulin-like growth factor IGF-1, (c) a variant of human serum albumin (HSA) at the C-terminus of the first peptide linker, wherein the variant of HSA comprises one or more mutations, wherein the one or more mutations comprise or consist of a substitution at one or more positions corresponding to C58 and N527, and combinations thereof, (d) a second peptide linker at the C-terminus of the variant of human serum albumin, and (e) a scFv having a binding specificity nephrin is at the C-terminus of the chimeric protein.

In some embodiments, the first linker comprises or consists of amino acid sequence is rich in glycine and serine residues. In some embodiments, the first linker comprises or consists of amino acid sequence (GSGGGSG)1-7 (SEQ ID NO: 23). In some embodiments, the second linker comprises or consists of comprises or consists of a tetratricopeptide repeat (TPR) domain. In some embodiments, the second linker comprises or consists of amino acids set forth in SEQ ID NO: 24, SEQ ID NO: 51, SEQ ID NO: 52, SEQ ID NO: 53 or SEQ ID NO: 54.

In some embodiments, the first linker comprises or consists of amino acid sequence (GSGGGSG)1-7 and the second linker comprises or consists of a tetratricopeptide repeat (TPR) domain. In some embodiments, the second linker comprises or consists of amino acids set forth in SEQ ID NO: 24, SEQ ID NO: 51, SEQ ID NO: 52, SEQ ID NO: 53 or SEQ ID NO: 54. In some embodiments, the second linker comprises or consists of amino acids set forth in SEQ ID NO: 24, SEQ ID NO: 51, SEQ ID NO: 52, SEQ ID NO: 53 or SEQ ID NO: 54. In some embodiments, the first linker comprises or consists of 1 to 7 repeats of amino acid sequence (GSGGGSG (SEQ ID NO: 23) and the second linker comprises or consists of amino acids set forth in SEQ ID NO: 51, SEQ ID NO: 52, SEQ ID NO: 53 or SEQ ID NO: 54.

### Nucleic Acids

Provided herein are polynucleotides encoding the chimeric proteins, polypeptide domains, activator domains, targeting domains, half-life modulators, peptides that may be in the form of RNA or in the form of DNA, which DNA includes cDNA and synthetic DNA. The DNA may be double-stranded or single-stranded. The coding sequences that encode the variants of the present disclosure may vary as a result of the redundancy or degeneracy of the genetic code.

### Pharmaceutical Compositions

Pharmaceutical compositions comprising a therapeutically effective amount of at least one chimeric protein as described herein, together with at least one physiologically acceptable carrier, are provided. Such compositions may be used for treating patients who are suffering from, or at risk for, tissue damage, in order to prevent tissue damage, or to repair or regenerate damaged tissue.

As used herein, the terms “physiologically acceptable” and “pharmaceutically acceptable” are used interchangeably and mean approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the chimeric protein is administered.

Physiologically acceptable carriers can be sterile liquids, such as water and oils, including those of petroleum, animal, vegetable or synthetic origin (e.g., peanut oil, soybean oil, mineral oil, or sesame oil). Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water and ethanol. The composition, if desired, can also contain minor amounts of wetting or emulsifying agents, or pH buffering agents.

Pharmaceutical compositions may be formulated for any appropriate manner of administration, including, for example, parenteral, intranasal, topical, oral, or local administration, such as by a transdermal means for prophylactic and/or therapeutic treatment. These compositions can take any of a variety of well-known forms that suit the mode of administration, such as solutions, suspensions, emulsions, tablets, pills, capsules, powders, aerosols and sustained-release formulations. The composition can be formulated as a suppository, with traditional binders and carriers. Oral formulation can include standard carriers such as pharmaceutical grades of mannitol, lactose, starch, magnesium stearate, sodium saccharine, cellulose, magnesium carbonate, etc. Examples of suitable pharmaceutical modes of administration and carriers are described in “Remington: The Science and Practice of Pharmacy,” A. R. Gennaro, ed. Lippincott Williams & Wilkins, Philadelphia, Pa. (21.sup.st ed., 2005).

In certain embodiments, pharmaceutical compositions are formulated in any conventional manner using one or more physiologically acceptable carriers comprising excipients and auxiliaries which facilitate processing of the chimeric protein into preparations which can be used pharmaceutically. Proper formulation is dependent upon the route of administration chosen.

The pharmaceutical compositions provided herein can be administered parenterally (e.g., by intravenous, intramuscular, or subcutaneous injection), or intra-nasally or intra-thecally or by oral ingestion or by topical application. For parenteral administration, the chimeric protein can either be suspended or dissolved in the carrier. A sterile aqueous carrier is generally preferred, such as water, buffered water, saline or phosphate-buffered saline. In addition, sterile, fixed oils may be employed as a solvent or suspending medium. For this purpose any bland fixed oil may be employed, including synthetic mono- or diglycerides. In addition, fatty acids such as oleic acid find use in the preparation of injectable compositions. Pharmaceutically acceptable auxiliary substances may also be included to approximate physiological conditions, such as pH adjusting and buffering agents, tonicity adjusting agents, dispersing agents, suspending agents, wetting agents, detergents, preservatives, local anesthetics and buffering agents.

In some embodiments, the pharmaceutical composition is formulated for intravenous administration to a patient (e.g., a human). Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a sealed (e.g., hermetically sealed) container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In some embodiments, compositions intended for oral use may be presented as, for example, tablets, troches, lozenges, aqueous or oily suspensions, dispersible powders or granules, emulsion, hard or soft capsules, or syrups or elixirs. Such compositions may further comprise one or more components such as sweetening agents flavoring agents, coloring agents and preserving agents. Tablets contain the active ingredient in admixture with physiologically acceptable excipients that are suitable for the manufacture of tablets. Such excipients include, for example, inert diluents, granulating and disintegrating agents, binding agents and lubricating agents. Formulations for oral use may also be presented as hard gelatin capsules wherein the active ingredient is mixed with an inert solid diluent, or as soft gelatin capsules wherein the active ingredient is mixed with water or an oil medium. Aqueous suspensions comprise the active materials in admixture with one or more excipients suitable for the manufacture of aqueous suspensions. Such excipients include suspending agents and dispersing or wetting agents. Dispersible powders and granules suitable for preparation of an aqueous suspension by the addition of water provide the active ingredient in admixture with a dispersing or wetting agent, suspending agent and one or more preservatives.

Oily suspensions may be formulated by suspending the active ingredients in a vegetable oil (e.g., arachis oil, olive oil, sesame oil or coconut oil) or in a mineral oil such as liquid paraffin. Pharmaceutical compositions may also be in the form of oil-in-water emulsions. The oily phase may be a vegetable oil or a mineral oil or mixture thereof. Suitable emulsifying agents include, for example, naturally-occurring gums, naturally-occurring phosphatides and anhydrides.

Pharmaceutical compositions may be sterilized by conventional sterilization techniques, or may be sterile filtered. Sterile aqueous solutions may be packaged for use as is, or lyophilized, the lyophilized preparation being combined with a sterile aqueous carrier prior to administration. The pH of an aqueous pharmaceutical composition typically will be between 3 and 11, more preferably between 5 and 9 or between 6 and 8, and most preferably between 7 and 8, such as 7 to 7.5.

In some embodiments the chimeric proteins provided herein are present within a pharmaceutical composition at a concentration such that administration of a single dose to a patient delivers a therapeutically effective amount. A therapeutically effective amount is an amount that results in a discernible patient benefit, such as detectable repair or regeneration of damaged tissue, prevention or diminution of symptoms of tissue damage.

Therapeutically effective amounts can be approximated from the amounts sufficient to achieve detectable tissue repair or regeneration in one or more animal models. Nonetheless, it will be apparent that a variety of factors will affect the therapeutically effective amount, including the activity of the chimeric protein employed; the age, body weight, general health, sex and diet of the patient; the time and route of administration; the rate of excretion; any simultaneous treatment, such as a drug combination; and the type and severity of the tissue damage in the patient undergoing treatment.

Optimal dosages may be established using routine testing, and procedures that are well known in the art. Dosages generally range from about 0.5 mg to about 5,000 mg of chimeric protein per dose (e.g., 0.5 mg, 1 mg, 2 mg, 5 mg, 10 mg, 50 mg, 100 mg, 500 mg, 1,000 mg, 1,500 mg, 2,000 mg, 3, 000 mg, 3,500 mg, 4,000 mg, 4,500 mg or 5,000 mg per dose). In general, compositions providing dosage levels ranging from about 0.1 mg to about 100 mg per kilogram of body weight per day are used. For example, about 0.1, 1, 5, 10, 20, 25, 30, 40, 50, 60, 70, 80, 90 or 100 mg per kilogram of body weight per day can be administered.

In some embodiments, the method comprises administering between 12 to 25 mg/kg, for example 12, 13, 14, 15, 16, 17, 18, 19, 20, 21, 22, 23, 24 or 25 mg/kg, to the subject in need thereof.

In some embodiments, the therapeutic concentration of the chimeric protein provides a serum/plasma concentration of about 100 ng/mL. In some embodiments, a concentration of 100 ng/mL can effectively drive pro-survival signaling (e.g. phosphorylation of AKT) in damaged tissues.

Pharmaceutical compositions may be packaged for treating or preventing tissue damage. Packaged pharmaceutical preparations include a container holding a therapeutically effective amount of at least one pharmaceutical composition as described herein and instructions (e.g., labeling) indicating that the contained composition is to be used for preventing or treating kidney disease in a patient. Pharmaceutical compositions may be packaged in multiple single dose units, each containing a fixed amount of chimeric protein in a sealed package. Alternatively, the container may hold multiple doses of the pharmaceutical composition.

### Methods of Treatment

The pharmaceutical compositions can be administered to a patient (e.g. a human) to treat tissue damage in the patient. As used herein the term “patient” or a “subject” as used interchangeably and refers to a mammal, preferably a human.

The term “therapeutically effective amount” refers to the amount or dose of chimeric proteins described herein which, upon single or multiple dose administration to a patient, provides the desired treatment.

In therapeutic applications, treatment is performed in order to reduce the severity of the pathological tissue damage or regenerate cells, tissue or organs after damage.

“Treatment”, “treat”, or “treating” as used herein refer to a method of reducing the effects or symptoms of a disease or condition, a method of reducing the disease or condition itself rather than just the symptoms, or a method of reducing tissue/organ damage. The treatment can be provided in order to reverse, alleviate, inhibit the progression of, prevent or reduce the likelihood of a disease, or in order to reverse, alleviate, inhibit or prevent the progression of, prevent or reduce the likelihood of one or more symptoms or manifestations of a disease. “Prevent” refers to causing a disease or symptom or manifestation of a disease not to occur for at least a period of time in at least some patients. The methods of treatment as used herein can refer to a 10%, 20%, 30%, 40%, 50%, 60%, 70%, 80%, 90%, 100% (or any amount therebetween) reduction in the severity of a symptom or disease progression when compared to levels in the same subject before treatment or control subjects. It is understood that “treatment” does not necessarily refer to a cure of the disease or condition, but an improvement in the outlook of a disease or condition.

In some embodiments, the treatment results in rehabilitation of ailing cells. In some embodiments, the treatment results in prevention of the spread of the damage to adjacent tissues.

Treating can include administering the chimeric protein or composition comprising the chimeric protein to the subject following the development of one or more symptoms or manifestations indicative of a disease, e.g., in order to reverse, alleviate, reduce the severity of, and/or inhibit or prevent the progression of the disease and/or to reverse, alleviate, reduce the severity of, and/or inhibit or one or more symptoms or manifestations of the disease. The chimeric protein or composition comprising the chimeric protein can be administered to a subject who has developed a disease, or is at increased risk of developing the disease relative to a member of the general population. The chimeric protein or composition comprising the chimeric protein can be administered to a subject who has developed a disease and is at increased risk of developing one or more particular symptoms or manifestations of the disease or an exacerbation of the disease relative to other individuals diagnosed with the disease, or relative to the subject's typical or average risk for such symptom or manifestation or exacerbation. The chimeric protein or composition comprising the chimeric protein can be administered prophylactically, i.e., before development of any symptom or manifestation of the disease.

As used herein, “reduction”, “decrease” or “reduce” refer to any change that results in a smaller amount of a symptom, condition, disease or tissue damage. For example, a reduction or decrease can be a change in the kidney disease treated such that the symptoms are less than previously observed. Thus, for example, a reduction or decrease can include but is not limited to a 10%, 20%, 30%, 40%, 50%, 60%, 70%, 80%, 90% or 100% (or any percentage of reduction in between) decrease in the symptoms associated with the kidney disease treated.

In some embodiments, a therapeutically effective amount of the composition comprising the chimeric protein reduces at least one symptom by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein reduces at least one symptom by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein disclosed herein reduces at least one symptom by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In some embodiments, a therapeutically effective amount of the chimeric protein reduces at least one symptom for, e.g., at least one week, at least one month, at least two months, at least three months, at least four months, at least five months, at least six months, at least seven months, at least eight months, at least nine months, at least ten months, at least eleven months, or at least twelve months.

In some embodiments, the pharmaceutical composition comprising an effective amount of the chimeric protein can be administered in combination with other therapeutic compositions.

In some embodiments, the pharmaceutical compositions can be administered in combination with existing treatments known in the art.

In some embodiments, a therapeutically effective amount generally is in the range of about 0.1 mg/kg to about 100.0 mg/kg per dose. In some embodiments, an effective amount of a protein disclosed herein can be, e.g., about 0.1 mg/kg to about 1 mg/kg per dose, 0.1 mg/kg to about 10 mg/kg per dose, 0.1 mg/kg to about 100 mg/kg per dose, 1 mg/kg to about 100 mg/kg per dose or 10 mg/kg to about 100 mg/kg per dose.

In some embodiments, a therapeutically effective amount generally is in the range of about 0.1 mg/kg to about 100.0 mg/kg per dose. In some embodiments, an effective amount of a protein disclosed herein can be, e.g., about 0.1 mg/kg to about 1 mg/kg per dose, 0.1 mg/kg to about 10 mg/kg per dose, 0.1 mg/kg to about 100 mg/kg per dose, 1 mg/kg to about 100 mg/kg per dose or 10 mg/kg to about 100 mg/kg per dose.

In some embodiments, a therapeutically effective amount generally is in the range of about 0.1 mg/kg to about 1,000.0 mg/kg per day. In some embodiments, an effective amount of a protein disclosed herein can be, e.g., about 0.01 mg/kg to about 0.1 mg/kg per day, 0.01 mg/kg to about 1 mg/kg per day, 0.01 mg/kg to about 10 mg/kg per day, 0.01 mg/kg to about 100.0 mg/kg per day, 0.01 mg/kg to about 200.0 mg/kg per day, 0.1 mg/kg to about 1 mg/kg per day, 0.1 mg/kg to about 10 mg/kg per day, 0.1 mg/kg to about 100 mg/kg per day, 0.1 mg/kg to about 200 mg/kg per day, 1 mg/kg to about 100 mg/kg per day, 1 mg/kg to about 200 mg/kg per day, 10 mg/kg to about 100 mg/kg per day or 10 mg/kg to about 1000 mg/kg per day.

Dosing can be single dosage or cumulative (serial dosing), and can be readily determined by one skilled in the art. For instance, dosing may comprise a one-time administration of an effective dose of the composition disclosed herein. As a non-limiting example, an effective dose of the composition disclosed herein can be administered once to a patient. Alternatively, dosing may comprise multiple administrations of an effective dose of the chimeric protein or pharmaceutical composition disclosed herein carried out over a range of time periods, such as, e.g., from one time daily to ten times daily. The duration of dosing may comprise single or multiple periods of multiple administrations of an effective dose of the chimeric protein or pharmaceutical composition disclosed herein carried out over a range of time periods, such as, e.g., from one day to one week, from one week to one month, from one month to one year, from one year to one decade, from one decade to multiple decades.

### Routes of Administration

Suitable routes of administration include, but are not limited to, parenteral administration. In addition, by way of example only, parenteral delivery includes intramuscular, subcutaneous, intravenous, intramedullary injections, intrathecal, intraperitoneal, intralymphatic, and intranasal injections.

In some embodiments, the pharmaceutical composition includes, but is not limited to, a composition suitable for oral, rectal, nasal, inhalation, topical (including, but not limited to, dermal, transdermal, buccal and sublingual), vaginal or parenteral (including, but not limited to, subcutaneous, intramuscular, intravenous, intradermal, intraperitoneal, intraocular and inhalation administration).

In some embodiments, the pharmaceutical composition is suitable for intravenous, intramuscular, subcutaneous, intranasal, intrathecal or intraperitoneal administration.

In some embodiments, the composition is administered by direct injection. In some embodiments, the composition is formulated for intravenous (IV) injection. In some embodiments, the composition is formulated for IV bolus injection.

### Indications:

### 1. Glomerulonephritis:

Glomerulonephritis is an umbrella term for kidney diseases characterized by inflammation of the glomeruli or of the small blood vessels in the kidney that can lead to nephrotic or nephritic syndromes. Glomerulonephritis results in a dysregulation of glomerular cells and may end up in chronic alterations and subsequent loss of renal function. There are non-proliferative and proliferative forms of glomerulonephritis. For example, in minimal change disease, a type of non-proliferative glomerulonephritis, the foot processes of podocytes in Bowman's capsule are lost. As a result, the structural barrier for filtering protein from the blood provided by healthy podocytes is disrupted. This leads to massive amounts of protein being lost from the blood to the urine in a patient (proteinuria). Glomerulonephritis can be the result of dysregulation of glomerular mesangial cells (e.g. mesangioproliferative IgA nephritis) or the damage of epithelial cells, in particular podocytes (e.g. diabetic nephropathy or membranous glomerulopathy). Mesangial cells are one major cell type of the glomerulus, together with podocytes and endothelial cells of the capillary tuft. In some embodiments, selective targeting of growth factor proteins to mesangial cells or podocytes may be useful to improve kidney function.

Additional diseases include: Minimal change disease, Focal segmental glomerulosclerosis, Membranous glomerulonephritis, Thin basement membrane disease, Fibronectin glomerulopathy, anti-GBM disease/Goodpasture's Disease, IgA Vasculitis.

### 2. Nephrotic Syndrome:

Nephrotic syndrome is a term used to characterize any disease due to kidney damage. Generally, nephrotic syndrome is caused by inflammation that allows proteins to pass through podocytes and appear in the urine. Selective targeting of growth factors to podocytes may be useful in repairing kidney damage and improve kidney function.

Additional diseases include: Primary childhood nephrotic syndrome, secondary childhood nephrotic syndrome, congenital nephrotic syndrome.

### 3. Chronic Kidney Disease.

Chronic kidney disease (CKD) is the gradual loss of kidney function over time (e.g., months-years). Generally, CKD is caused by kidney damage and inflammation as discussed above. When the glomeruli are inflamed, proteins such as albumin pass through podocytes leading to edema and liver dysfunction Selective targeting of growth factors to podocytes may be useful in repairing kidney damage and improve kidney function.

Additional diseases include: All of the above listed diseases, hemolytic uremic syndrome, Polycystic kidney disease.

### 4. Diabetic Nephropathy

Diabetic nephropathy is the chronic loss of kidney function due to diabetic hyperglycemia and/or ketoacidosis. It is due to longstanding diabetes mellitus. The lack of controlled blood glucose can cause multiple changes in the kidney, including thickening of the basement membrane, widening of the slit membranes of podocytes, and lack of filtration.

Selective targeting of growth factors to podocytes may be useful in repairing and promoting podocyte growth to improve filtration and kidney function.

### 5. Acute Kidney Injury (AKI)

Acute kidney injury (AKI), is linked to adverse long-term outcomes, including permanent renal impairment and end-stage renal disease. In some embodiments, the pharmaceutical composition can be used to prevent or protect kidney tissue from damage and/or to regenerate tissue and/or blood supply after kidney damage or tissue damage.

### EXAMPLES

The following Examples are offered by way of illustration and not by way of limitation. Unless otherwise specified, all reagents and solvents are of standard commercial grade and are used without further purification. Using routine modifications, the procedures provided in the following Examples may be varied by those of ordinary skill in the art to make and use other chimeric proteins and pharmaceutical compositions within the scope of the present disclosure.

### Example 1: Prototype sequence

Scp801 (see FIG. 2A and FIG. 2B, SEQ ID NO: 41) was designed as a prototype for the chimeric protein according to some embodiments. The anti-TnI scFv used in Scp801 was determined to be well-structured based upon publicly available crystallographic data.

Scp801 was purified by affinity chromatography.

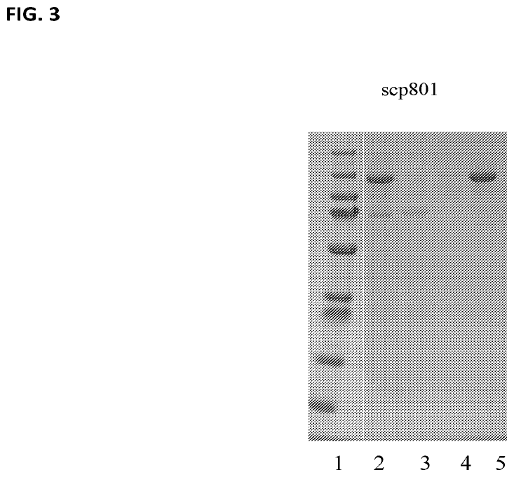

Process intermediates and wash fractions obtained in the purification of scp801 are loaded to a SDS-PAGE gel. For scp801, the lanes on the gel, from left to right, are: 1. Molecular weight ladder; 2. Clarified harvest for scp801; 3. Flow through fraction from affinity chromatography; 4. Wash fraction from affinity chromatography; and 5. Elution fraction from affinity chromatography. See FIG. 3.

### Example 2: Synthesis of Chimeric Proteins

Desired amino acid sequences of chimeric proteins were reverse translated to nucleotide sequences for expression in HEK293 cells. Briefly, plasmid DNA encoding a secretion signal sequence and the chimeric protein was chemically transfected to HEK293 cells grown in suspension culture. Following at least 72 hours of expression, the cells were separated from the media. Expression level was analyzed by SDS-PAGE gel and the cell culture media containing the expressed and secreted chimeric protein was designated for purification.

### Example 3: Purification of Chimeric Proteins

Cell culture media containing the expressed chimeric protein was loaded to an affinity resin (blue ligand) to capture the HSA containing chimeric proteins. The affinity chromatography was run in bind, wash, and elute mode, whereby chimeric protein bound to the affinity column was washed with high salt buffers to disrupt non-specific electrostatic interactions before the desired chimeric protein product was eluted by addition of sodium caprylate containing buffer. Following chromatography, the load, wash, elution and strip fractions were analyzed by SDS PAGE gel to check purity and the elution fraction was analyzed by size exclusion chromatography to analyze the percent monomer. The elution fraction was then diafiltered to a standard buffer for protein storage (50 mM Tromethamine, 50 mM NaCl, pH 7.5) and the material was concentrated to about 20 mg/mL. For all chimeric proteins tested comprising the lk7-HSA-cTPR3 scaffold, purity following the single affinity chromatography step exceeded 95% purity by SDS-PAGE and 85% monomer by size exclusion chromatography. In contrast to other therapeutic proteins containing Serum Albumin, the overall yield, process performance, and monomer content is surprising. All six constructs (scp801-scp806) were identical in sequence for the Igf1-lk7-HSA-cTPR3 portion and possessed different targeting domain sequences for the different single chain antibodies.

Prior attempts to express and purify scFv containing chimeric proteins that used short flexible linkers (i.e., 1k7) to connect the half-life modulator to the scFv targeting domain failed to produce material at the expression stage. See FIG. 3.

### Example 4: Localization of IGF-1 E3R/Y31 Å-HSA-Anti-PTPRO ScFv in Rat Kidney

Healthy, male Sprague-Dawley rats aged 8 weeks were injected IV with targeted (scp802, scp806) or untargeted (also referred as non-targeted control) (scp207) Igf1-HSA variants.

The dose for each molecule was 25 mg/kg (1.25 mL/kg) in a formulation buffer containing 50 mM Tris, 50 mM NaCl, pH 7.5. Rats were sacrificed 2 hours post dose and kidneys were harvested and fixed in neutral buffered formalin. Fixed kidneys were paraffin embedded and 5 μm thick longitudinal sections were stained for the presence of HSA containing molecules using standard immunohistochemical techniques with a rabbit anti-HSA polyclonal antibody (Abcam ab2406) and an anti-rabbit secondary detection reagent. Whole slide scans were performed at 20× magnification, and additional digital zoom snapshots were captured at up to 1200× magnification in QuPath version 0.3.1.

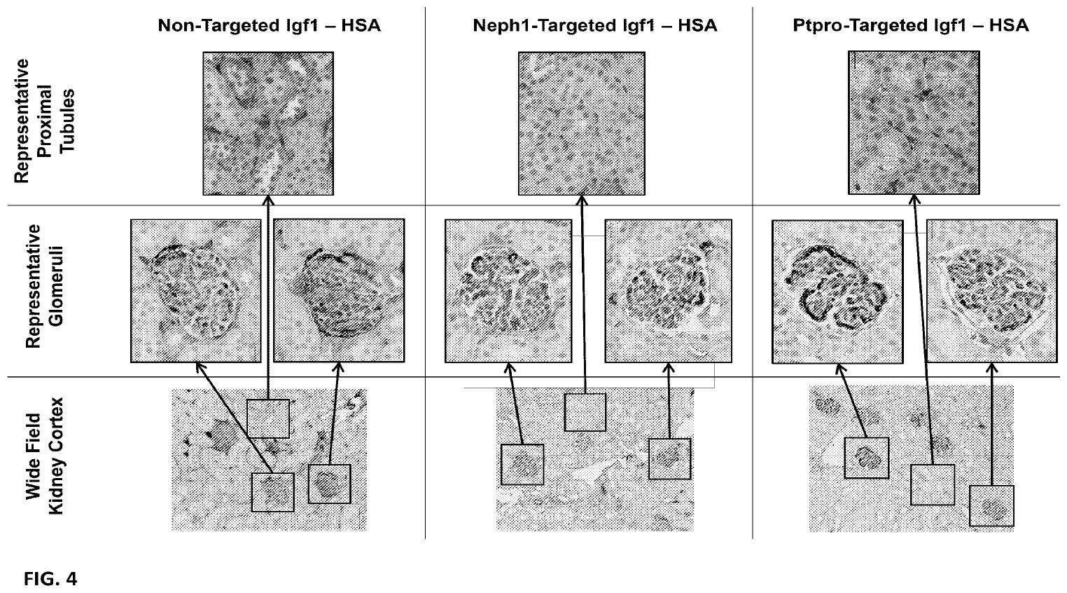

FIG. 4 shows detection of different fusion proteins in the cortex of healthy rat kidney using anti-HSA immunohistochemistry (IHC) staining for non-targeted control scp207 (IGF-1 E3R-HSA, SEQ ID NO: 47), targeted growth factor scp802 (IGF-1 E3R/Y31 Å-HSA-anti-PTPRO, SEQ ID NO: 42), and targeted growth factor scp806 (IGF-1 E3R/Y31 Å-HSA-anti-Neph1, SEQ ID NO: 46). The cortex is shown at 200× magnification in the bottom row, while representative images of proximal tubules and individual glomeruli are shown at 1,200× magnification in the top and middle rows respectively. For the non-targeted control, diffuse staining is evident outside of the glomeruli. For the targeted growth factors, regions outside of the glomeruli are less intensely stained; the relatively intense staining of the glomeruli for the targeted growth factors reveals preferential distribution of these constructs to the glomeruli, consistent with known expression of these target molecules (PTPRO and Neph1).

Both targeted and untargeted Igf1-HSA variants were detected in rat kidney by IHC 2 hours post IV injection. The targeted variants (scp802, scp806) were detected primarily in glomerular capillaries and in the papilla, with very limited staining of proximal tubules and interstitial microvasculature. In contrast, the untargeted scp207 was detected at low levels on the apical surface of proximal tubules, in interstitial microvasculature, in the papilla, and diffusely across glomeruli including strong staining in the Bowman's space. See FIG. 4. The staining patterns observed suggest that the PTPRO and Nephrin targeted ScFv arms of scp802 and scp806 drive specific localization to glomerular substructures. The data also suggest that that the untargeted Igf1-HSA variant scp207 may be filtered by the kidney at a higher level than the targeted variants.

SEQ ID NO: 42 is the amino acid sequence of
scp802.
GPRTLCGAELVDALQFVCGDRGFYFNKPTGAGSSSRRAPQTGIVDECCFR
SCDLRRLEMYCAPLKPAKSAGSGGGSGDAHKSEVAHRFKDLGEENFKALV
LIAFAQYLQQSPFEDHVKLVNEVTEFAKTCVADESAENCDKSLHTLFGDK
LCTVATLRETYGEMADCCAKQEPERNECFLQHKDDNPNLPRLVRPEVDVM
CTAFHDNEETFLKKYLYEIARRHPYFYAPELLFFAKRYKAAFTECCQAAD
KAACLLPKLDELRDEGKASSAKQRLKCASLQKFGERAFKAWAVARLSQRF
PKAEFAEVSKLVTDLTKVHTECCHGDLLECADDRADLAKYICENQDSISS
KLKECCEKPLLEKSHCIAEVENDEMPADLPSLAADFVESKDVCKNYAEAK
DVFLGMFLYEYARRHPDYSVVLLLRLAKTYETTLEKCCAAADPHECYAKV
EDEFKPLVEEPQNLIKQNCELFEQLGEYKFQNALLVRYTKKVPQVSTPTL
VEVSRNLGKVGSKCCKHPEAKRMPCAEDYLSVVLNQLCVLHEKTPVSDRV
TKCCTESLVNRRPCFSALEVDETYVPKEFQAETFTFHADICTLSEKERQI
KKQTALVELVKHKPKATKEQLKAVMDDFAAFVEKCCKADDKETCFAEEGK
KLVAASQAALGLKLSGGGGSGGGGSGGGGSAEAWYNLGNAYYKQGDYQKA
IEYYQKALELDPNNAEAWYNLGNAYYKQGDYQKAIEYYQKALELDPNNAE
AWYNLGNAYYKQGDYQKAIEDYQKALELDPNNLQRSAGGGGSGGGGSGGG
GASQVQLQQSGGGLVQPGRSLRLSCEASGFTFSSYAMSWVRQAPGKGLEW
VSAISGSGGSTYYADSVKGRFTISRDNSKNTLYLQMNSLRAEDTAVYYCA
KPLGDLYVNDAFDIWGQGTMVTVSSGGGGSGGGGSGGGGSQSVLTQPPSV
SGAPGQRVTISCTGSSSNIGAGYDVHWYQQLPGTAPKLMIYEVSNRPSGV
SNRFSGSKSGNTASLTISGLQTEDEAYYYCSSYASGNIVVFGGGTKLTVL
G
SEQ ID NO: 46 is the amino acid sequence of
scp806.
GPRTLCGAELVDALQFVCGDRGFYFNKPTGAGSSSRRAPQTGIVDECCFR
SCDLRRLEMYCAPLKPAKSAGSGGGSGDAHKSEVAHRFKDLGEENFKALV
LIAFAQYLQQSPFEDHVKLVNEVTEFAKTCVADESAENCDKSLHTLFGDK
LCTVATLRETYGEMADCCAKQEPERNECFLQHKDDNPNLPRLVRPEVDVM
CTAFHDNEETFLKKYLYEIARRHPYFYAPELLFFAKRYKAAFTECCQAAD
KAACLLPKLDELRDEGKASSAKQRLKCASLQKFGERAFKAWAVARLSQRF
PKAEFAEVSKLVTDLTKVHTECCHGDLLECADDRADLAKYICENQDSISS
KLKECCEKPLLEKSHCIAEVENDEMPADLPSLAADFVESKDVCKNYAEAK
DVELGMELYEYARRHPDYSVVLLLRLAKTYETTLEKCCAAADPHECYAKV
EDEFKPLVEEPQNLIKQNCELFEQLGEYKFQNALLVRYTKKVPQVSTPTL
VEVSRNLGKVGSKCCKHPEAKRMPCAEDYLSVVLNQLCVLHEKTPVSDRV
TKCCTESLVNRRPCFSALEVDETYVPKEFQAETFTFHADICTLSEKERQI
KKQTALVELVKHKPKATKEQLKAVMDDFAAFVEKCCKADDKETCFAEEGK
KLVAASQAALGLKLSGGGGSGGGGSGGGGSAEAWYNLGNAYYKQGDYQKA
IEYYQKALELDPNNAEAWYNLGNAYYKQGDYQKAIEYYQKALELDPNNAE
AWYNLGNAYYKQGDYQKAIEDYQKALELDPNNLQRSAGGGGSGGGGSGGG
GASQVQLQQSGGGLVQPGGSLRLSCAASGFTFSIYAMSWVRQAPGKGLEW
VSAISDSGTGTYYADSVKGRFTISRDNSKNTLYLQMNSLTAEDTAVYYCA
RRSGNYYLDYWGQGTLVTVSSGGGGSGGGGSGGGGSDIVMTQSPSTLSAS
IGDRVTITCRASQSISSWLAWYQQKPGKAPKLLIHKTSNLESGVPSRFSG
SGSGTEFSLTISSLQPDDFATYYCQQYYSYPWTFGRGTKLEIKR
SEQ ID NO: 47 is the amino acid sequence of
scp207.
GPRTLCGAELVDALQFVCGDRGFYENKPTGYGSSSRRAPQTGIVDECCFR
SCDLRRLEMYCAPLKPAKSAGSGGGSGDAHKSEVAHRFKDLGEENFKALV
LIAFAQYLQQSPFEDHVKLVNEVTEFAKTCVADESAENCDKSLHTLFGDK
LCTVATLRETYGEMADCCAKQEPERNECFLQHKDDNPNLPRLVRPEVDVM
CTAFHDNEETFLKKYLYEIARRHPYFYAPELLFFAKRYKAAFTECCQAAD
KAACLLPKLDELRDEGKASSAKQRLKCASLQKFGERAFKAWAVARLSQRF
PKAEFAEVSKLVTDLTKVHTECCHGDLLECADDRADLAKYICENQDSISS
KLKECCEKPLLEKSHCIAEVENDEMPADLPSLAADFVESKDVCKNYAEAK
DVELGMELYEYARRHPDYSVVLLLRLAKTYETTLEKCCAAADPHECYAKV
EDEFKPLVEEPQNLIKQNCELFEQLGEYKFQNALLVRYTKKVPQVSTPTL
VEVSRNLGKVGSKCCKHPEAKRMPCAEDYLSVVLNQLCVLHEKTPVSDRV
TKCCTESLVNRRPCFSALEVDETYVPKEFQAETFTFHADICTLSEKERQI
KKQTALVELVKHKPKATKEQLKAVMDDFAAFVEKCCKADDKETCFAEEGK
KLVAASQAALGL

### Example 5: Safety and Efficacy Study

Safety and efficacy of putative designs will be tested in rodent models first. For safety, kidney function with be tested to ensure that glomerular filtration rate (GFR) is maintained with these designs.

The primary attribute of engineered chimeric proteins that will be optimized for is the selective accumulation to the glomeruli of healthy kidneys. Immunohistochemistry will be employed for the detection of engineered chimeric proteins in the glomeruli.

Designs that selectively accumulate in the glomeruli of healthy kidneys will subsequently be tested to ensure that normal kidney function is not perturbed. Kidney function will be monitored by serum creatinine and blood urea nitrogen (BUN). Designs that do not impair renal function will be advanced to efficacy studies.

Efficacy studies will be conducted in rodent models of kidney disease with symptomology consistent with renal impairment. The primary signal for appraisal of efficacy will be the improvement of kidney function as assessed by serum creatinine and BUN levels.

Efficacy studies will also assess the morphology and the number of glomeruli as alterations in the morphology and numbers of glomeruli and glomerular cells occur in various renal disorders.

### INCORPORATION BY REFERENCE

All publications, patents and sequence database entries mentioned herein are hereby incorporated by reference in their entirety as if each individual publication or patent was specifically and individually indicated to be incorporated by reference.

What is claimed is:

1. A chimeric protein comprising:
(a) a targeting domain comprising a scFv having a binding specificity to a cell surface protein of a kidney tissue, and
(b) an activator domain comprising a growth factor, wherein the targeting domain is at the C-terminus and the activator domain is at the N-terminus of the chimeric protein.

2. A chimeric protein comprising:
(a) a targeting domain comprising a scFv having a binding specificity to a cell surface protein of a kidney tissue, and
(b) an activator domain comprising a growth factor, wherein the targeting domain is at the N-terminus and the activator domain is at the C-terminus of the chimeric protein.

3. The chimeric protein of claim 1 or claim 2 further comprising one or more peptide linkers between the targeting domain and the activator domain.

4. The chimeric protein of claim 1 or claim 2 further comprising a half-life modulator between the targeting domain and the activator domain.

5. The chimeric protein of claim 4 further comprising a peptide linker between the targeting domain and the half-life modulator.

6. The chimeric protein of claim 4 or claim 5 further comprising a peptide linker between the activator domain and half-life modulator.

7. The chimeric protein of claim 4, wherein the half-life modulator extends the half-life of the chimeric protein.

8. The chimeric protein of claim 1 wherein the targeting domain targets cell surface protein of a glomerulus.

9. The chimeric protein of claim 8, wherein the targeting domain has a binding specificity to a protein displayed at the cell surface of podocytes, parietal epithelial cells, or glycocalyx-coated fenestrated endothelial cells of the kidney.

10. The chimeric protein of claim 8, wherein the targeting domain has a binding specificity to Protein Tyrosine Phosphatase Receptor Type O (PTPRO), Podocin, Phospholipase A2 Receptor 1, Integrin Subunit alpha 8, Apoptosis resistant E3 ubiquitin protein ligase 1, Integrin Subunit alpha 8, Apoptosis resistant E3 ubiquitin protein ligase 1, Podocalyxin, Synaptopodin, Alpha 3 integrin, Cysteine rich transmembrane BMP regulator 1, FAT atypical cadherin 1, NEPH1, Dystroglycan, or Podoplanin.

11. The chimeric protein of any one of claims 1-10, wherein the activator domain comprises or consists of IGF-1, IGF-2, Neuregulin1α, Neuregulin1β, BMP2, BMP7, FGF2, FGF9 VEGF-A or variant thereof or fragment thereof.

12. The chimeric protein of claim 5, wherein the peptide linker between the targeting domain and the half-life modulator comprises or consists of a tetratricopeptide repeat (TPR) domain.

13. The chimeric protein of claim 6 or claim 12, wherein the peptide linker between the half-life modulator and the activator domain is glycine, serine or glycine and serine rich.

14. A chimeric protein comprising:
(a) a scFv having a binding specificity to Protein Tyrosine Phosphatase Receptor Type O, and
(b) a variant of human insulin-like growth factor IGF-1, wherein the variant of IGF-1 comprises one or more mutations, wherein the one or more mutations consist of a substitution at one or more positions corresponding to E3, Y24, Y31, Y60, and combinations thereof,
wherein the scFv is at the C-terminus and the variant of human insulin-like growth factor IGF-1 is at the N-terminus of the chimeric protein.

15. A chimeric protein comprising:
(a) a scFv having a binding specificity to Protein Tyrosine Phosphatase Receptor Type O,
(b) a variant of human insulin-like growth factor IGF-1, wherein the variant of IGF-1 comprises one or more mutations, wherein the one or more mutations comprise or consist of a substitution at one or more positions corresponding to E3, Y24, Y31, Y60, and combinations thereof, and
(c) a variant of human serum albumin (HSA), wherein the variant of HSA comprises one or more mutations, wherein the one or more mutations comprise or consist of a substitution at one or more positions corresponding to C58 and N527, and combinations thereof,
wherein the scFv is at the C-terminus and the variant of human insulin-like growth factor IGF-1 is at the N-terminus of the chimeric protein.

16. A chimeric protein comprising:
(a) a variant of human insulin-like growth factor IGF-1, wherein the variant of IGF-1 comprises one or more mutations, wherein the one or more mutations comprise or consist of a substitution at one or more positions corresponding to E3, Y24, Y31, Y60, and combinations thereof at the N-terminus of the chimeric protein,
(b) a first peptide linker at the C-terminus of the variant of human insulin-like growth factor IGF-1,
(c) a variant of human serum albumin (HSA) at the C-terminus of the first peptide linker, wherein the variant of HSA comprises one or more mutations, wherein the one or more mutations comprise or consist of a substitution at one or more positions corresponding to C58 and N527, and combinations thereof,
(d) a second peptide linker at the C-terminus of the variant of human serum albumin,
(e) a scFv having a binding specificity to Protein Tyrosine Phosphatase Receptor Type O is at the C-terminus of the chimeric protein.

17. The chimeric protein of claim 16, wherein the first linker comprises or consists of amino acid sequence (GSGGGSG)1-7 and wherein the second linker comprises or consists of a tetratricopeptide repeat (TPR) domain.

18. A pharmaceutical composition comprising the chimeric protein of any one of the preceding claims and at least one physiologically acceptable carrier.

19. A method for treating kidney disease comprising administering the chimeric protein of claims 1-17 or the pharmaceutical composition of claim 18 to a subject in need thereof.

20. The method of claim 19, wherein the kidney disease is glomerulonephritis, nephrotic syndrome, chronic kidney disease, diabetic nephropathy or acute kidney injury.

21. The method of claim 19 or claim 20, wherein the chimeric protein or the pharmaceutical composition is administered intravenously, subcutaneously, or intraperitoneally to the subject in need thereof.

22. The method of claim 19 or claim 20, wherein the administering results in improvement of the subject's kidney function as assessed by serum creatinine and blood ureanitrogen levels.

23. A nucleic acid encoding the chimeric protein of any one of claims 1-17.

24. A polypeptide domain comprising a half-life modulator with a first peptide linker at its N-terminus and a second peptide linker at its C-terminus, wherein the first peptide linker comprises or consists of an amino acid sequence consisting of glycine and/or serine, wherein the second linker comprises or consists of a tetratricopeptide repeat (TPR) domain.

25. The polypeptide domain of claim 24, wherein the half-life modulator is variant of human serum albumin (HSA).

26. The polypeptide domain of claim 25, wherein the HSA comprises or consists of amino acid sequence set forth in SEQ ID NOs: 13-15.

27. The polypeptide domain of claim 24, wherein the first peptide linker comprises or consists of amino acid sequence set forth in SEQ ID NO: 23.

28. The polypeptide domain of any one of claims 24-26, wherein the second peptide linker comprises or consists of amino acid sequence set forth in SEQ ID NO: 24, SEQ ID NO:
51, SEQ ID NO: 52, SEQ ID NO: 53 or SEQ ID NO: 54.

29. The polypeptide domain of claim 24 having an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence set forth SEQ ID NO:
55, SEQ ID NO: 56, SEQ ID NO: 57, or SEQ ID NO: 58.

30. A nucleic acid encoding the polypeptide domain of any one of claims 24-29.

31. A polypeptide domain comprising a half-life modulator with a first peptide linker at its N-terminus and a second peptide linker at its C-terminus, wherein the first peptide linker comprises or consists of a tetratricopeptide repeat (TPR) domain, wherein the second linker comprises or consists of an amino acid sequence consisting of glycine and/or serine.

32. The polypeptide domain of claim 31, wherein the half-life modulator is variant of human serum albumin (HSA).

33. The polypeptide domain of claim 32, wherein the HSA comprises or consists of amino acid sequence set forth in SEQ ID NOs: 13-15.

34. The polypeptide domain of claim 32, wherein the second peptide linker comprises or consists of amino acid sequence set forth in SEQ ID NO: 23.

35. The polypeptide domain of any one of claims 31-34, wherein the first peptide linker comprises or consists of amino acid sequence set forth in SEQ ID NO: 24, SEQ ID NO:
51, SEQ ID NO: 52, SEQ ID NO: 53 or SEQ ID NO: 54.

36. A nucleic acid encoding the polypeptide domain of any one of claims 31-35.
